SEQUENCE LISTING (156 sequences)

SEQ ID NO: 1 - DNA, 558 bp, synthetic construct
  misc_feature 1..558: note = Mycobacterium smegmatis porin A mutant (D90N/D91N/D93N/D118R/D134R/E139K)
  source 1..558: mol_type = other DNA
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, synthetic construct
  REGION 1..184: note = Mycobacterium smegmatis porin A mutant (D90N/D91N/D93N/D118R/D134R/E139K)
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 885 bp, synthetic construct
  misc_feature 1..885: note = alpha-hemolysin mutant E111N/K147N
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = alpha-hemolysin mutant E111N/K147N
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, unidentified
  source 1..184: note = Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - protein, 8 aa, synthetic construct
  REGION 1..8: note = Amino acid sequence of the Hel308 motif
  VARIANT 2: note = X = C, M or L
  VARIANT 3: note = X = any amino acid
QXXGRAGR

SEQ ID NO: 9 - protein, 9 aa, synthetic construct
  REGION 1..9: note = Amino acid sequence of the extended Hel308 motif
  VARIANT 2: note = X = C, M or L
  VARIANT 3: note = X = any amino acid
QXXGRAGRP

SEQ ID NO: 10 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 11 - protein, 8 aa, synthetic construct
  REGION 1..8: note = Exemplary Hel308 motif
QMAGRAGR

SEQ ID NO: 12 - protein, 9 aa, synthetic construct
  REGION 1..9: note = Exemplary extended Hel308 motif
QMAGRAGRP

SEQ ID NO: 13 - protein, 720 aa, Pyrococcus furiosus
MRVDELRVDERIKSTLKERGIESFYPPQAEALKSGILEGKNALISIPTASGKTLIAEIAMVHRILTQGGKAVYIVPLKALAEEKFQEFQDWEKIGLRVAMATGDYDSKDEWLGKYDIIIATAEKFDSLLRHGSSWIKDVKILVADEIHLIGSRDRGATLEVILAHMLGKAQIIGLSATIGNPEELAEWLNAELIVSDWRPVKLRRGVFYQGFVTWEDGSIDRFSSWEELVYDAIRKKKGALIFVNMRRKAERVALELSKKVKSLLTKPEIRALNELADSLEENPTNEKLAKAIRGGVAFHHAGLGRDERVLVEENFRKGIIKAVVATPTLSAGINTPAFRVIIRDIWRYSDFGMERIPIIEVHQMLGRAGRPKYDEVGEGIIVSTSDDPREVMNHYIFGKPEKLFSQLSNESNLRSQVLALIATFGYSTVEEILKFISNTFYAYQRKDTYSLEEKIRNILYFLLENEFIEISLEDKIRPLSLGIRTAKLYIDPYTAKMFKDKMEEVVKDPNPIGIFHLISLTPDITPFNYSKREFERLEEEYYEFKDRLYFDDPYISGYDPYLERKFFRAFKTALVLLAWINEVPEGEIVEKYSVEPGDIYRIVETAEWLVYSLKEIAKVLGAYEIVDYLETLRVRVKYGIREELIPLMQLPLVGRRRARALYNSGFRSIEDISQARPEELLKIEGIGVKTVEAIFKFLGKNVKISEKPRKSTLDYFLKS

SEQ ID NO: 14 - protein, 8 aa, synthetic construct
  REGION 1..8: note = Exemplary Hel308 motif
QMLGRAGR

SEQ ID NO: 15 - protein, 9 aa, synthetic construct
  REGION 1..9: note = Exemplary extended Hel308 motif
QMLGRAGRP

SEQ ID NO: 16 - protein, 829 aa, Haloferax volcanii
MRTADLTGLPTGIPEALRDEGIEELYPPQAEAVEAGLTDGESLVAAVPTASGKTLIAELAMLSSVARGGKALYIVPLRALASEKKAEFERWEEYGIDVGVSTGNYESDGEWLSSRDIIVATSEKVDSLVRNNAAWMDQLTCVVADEVHLVDDRHRGPTLEVTLAKLRRLNTNLQVVALSATVGNAGVVSDWLDAELVKSDWRPIDLKMGVHYGNAVSFADGSQREVPVGRGERQTPALVADALEGDGEGDQGSSLVFVNSRRNAESAARRMADVTERYVTGDERSDLAELAAEIRDVSDTETSDDLANAVAKGAAFHHAGLAAEHRTLVEDAFRDRLIKCICATPTLAAGVNTPSRRVVVRDWQRYDGDYGGMKPLDVLEVHQMMGRAGRPGLDPYGEAVLLAKDADARDELFERYIWADAEDVRSKLAAEPALRTHLLATVASGFAHTREGLLEFLDQTLYATQTDDPERLGQVTDRVLDYLEVNGFVEFEGETIQATPVGHTVSRLYLDPMSAAEIIDGLEWAADHRTEKLRALAGETPEKPTRDRSESDESGGFQRASEMVADDGDGGGGEDGVGANGDGDSDDADGVETDRTYPTPLGLYHLVCRTPDMYQLYLKSGDRETYTELCYEREPEFLGRVPSEYEDVAFEDWLSALKTAKLLEDWVGEVDEDRITERYGVGPGDIRGKVETSEWLLGAAERLATELDLDSVYAVREAKKRVEYGVREELLDLAGVRGVGRKRARRLFEAGVETRADLREADKPRVLAALRGRRKTAENILEAAGRKDPSMDAVDEDDAPDDAVPDDAGFETAKERADQQASLGDFEGS

SEQ ID NO: 17 - protein, 8 aa, synthetic construct
  REGION 1..8: note = Exemplary Hel308 motif
QMMGRAGR

SEQ ID NO: 18 - protein, 9 aa, synthetic construct
  REGION 1..9: note = Exemplary extended Hel308 motif
QMMGRAGRP

SEQ ID NO: 19 - protein, 824 aa, Halorubrum lacusprofundi
MQPSSLSGLPAGVGEALEAEGVAELYPPQEAAVEAGVADGESLVAAVPTASGKTLIAELAMLSSIERGGKALYIVPLRALASEKKTEFERWEEFGVTVGVSTGNYESDGEWLATRDIIVATSEKVDSLIRNGAPWIDDLTCVVSDEVHLVDDPNRGPTLEVTLAKLRKVNPGLQTVALSATVGNADVIAEWLDAELVESDWRPIDLRMGVHFGNAIDFADGSKREVPVERGEDQTARLVADALDTEEDGQGGSSLVFVNSRRNAESSARKLTDVTGPRLTDDERDQLRELADEIRSGSDTDTASDLADAVEQGSAFHHAGLRSEDRARVEDAFRDRLIKCISATPTLAAGVNTPARRVIVRDWRRYDGEFGGMKPLDVLEVHQMCGRAGRPGLDPYGEAVLLANDADTKEELFERYLWADPEPVRSKLAAEPALRTHVLATVASGFASTRDGLLSFLDNTLYATQTDDEGRLAAVTDTVLDYLAVNDFIERDRDGGSESLTATGIGHTVSRLYLDPMSAAEMIDGLRSVARDAADTGASAEADNGEFVRTGDADDASGGDEPGFGTYTRAGDDESGERETENEETDEEETEASEVTPLGLYHLISRTPDMYELYLKSGDRETYTELCYERETEFLGDVPSEYEDVRFEDWLASLKTARLLEDWVNEVDEDRITERYGVGPGDIRGKVDTAEWLLRAAETLARDVEGVDGDVVVAVREARKRIEYGVREELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 20 - protein, 8 aa, synthetic construct
  REGION 1..8: note = Exemplary Hel308 motif
QMCGRAGR

SEQ ID NO: 21 - protein, 9 aa, synthetic construct
  REGION 1..9: note = Exemplary extended Hel308 motif
QMCGRAGRP

SEQ ID NO: 22 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 23 - protein, 8 aa, synthetic construct
  REGION 1..8: note = Exemplary Hel308 motif
QLCGRAGR

SEQ ID NO: 24 - protein, 9 aa, synthetic construct
  REGION 1..9: note = Exemplary extended Hel308 motif
QLCGRAGRP

SEQ ID NO: 25 - protein, 715 aa, Sulfolobus solfataricus
MSLELEWMPIEDLKLPSNVIEIIKKRGIKKLNPPQTEAVKKGLLEGNRLLLTSPTGSGKTLIAEMGIISFLLKNGGKAIYVTPLRALTNEKYLTFKDWELIGFKVAMTSGDYDTDDAWLKNYDIIITTYEKLDSLWRHRPEWLNEVNYFVLDELHYLNDPERGPVVESVTIRAKRRNLLALSATISNYKQIAKWLGAEPVATNWRPVPLIEGVIYPERKKKEYNVIFKDNTTKKVHGDDAIIAYTLDSLSKNGQVLVFRNSRKMAESTALKIANYMNFVSLDENALSEILKQLDDIEEGGSDEKELLKSLISKGVAYHHAGLSKALRDLIEEGFRQRKIKVIVATPTLAAGVNLPARTVIIGDIYRFNKKIAGYYDEIPIMEYKQMSGRAGRPGFDQIGESIVVVRDKEDVDRVFKKYVLSDVEPIESKLGSERAFYTFLLGILSAEGNLSEKQLENFAYESLLAKQLVDVYFDRAIRWLLEHSFIKEEGNTFALTNFGKRVADLYINPFTADIIRKGLEGHKASCELAYLHLLAFTPDGPLVSVGRNEEEELIELLEDLDCELLIEEPYEEDEYSLYINALKVALIMKDWMDEVDEDTILSKYNIGSGDLRNMVETMDWLTYSAYHLSRELKLNEHADKLRILNLRVRDGIKEELLELVQISGVGRKRARLLYNNGIKELGDVVMNPDKVKNLLGQKLGEKVVQEAARLLNRFH

SEQ ID NO: 26 - protein, 8 aa, synthetic construct
  REGION 1..8: note = Exemplary Hel308 motif
QMSGRAGR

SEQ ID NO: 27 - protein, 9 aa, synthetic construct
  REGION 1..9: note = Exemplary extended Hel308 motif
QMSGRAGRP

SEQ ID NO: 28 - protein, 685 aa, Methanogenium frigidum
MDLSLPKAFIQYYKDKGIESLYPPQSECIENGLLDGADLLVAIPTASGKTLIAEMAMHAAIARGGMCLYIVPLKALATEKAQEFKGKGAEIGVATGDYDQKEKRLGSNDIVIATSEKVDSLLRNGVPWLSQVTCLVVDEVHLIDDESRGPTLEMVITKLRHASPDMQVIGLSATIGNPKELAGWLGADLITSDWRPVDLREGICYHNTIYFDNEDKEIPAPAKTEDINLLLDCVADGGQCLVFVSSRRNAEGYAKRAATALKCSHAALDSIAEKLEAAAETDMGRVLATCVKKGAAFHHAGMNRMQRTLVEGGFRDGFIKSISSTPTLAAGLNLPARRVIIRDYLRYSGGEGMRPIPVREYRQMAGRAGRPHLDPYGEAILIAKTEYAVNDLHEEYVEAPDEDVTSRCGEKGVLTAHILSLIATGYARSYDELMAFLEKTLYAYQHTGKKALTRTLDDALGFLTEAEMVTDLSGMLHATEYGDLTSRLYIDPHSAEIITTALREEGELTDLALLQLLCMTPDMFTLYVKKNDLGTLEKFFFEHEEEFRTEFSYDEMEDFFRSLKTAMLLSDWTDEIGDDTICTRFGVGPGDIFNAVQGISWLLHASGRLARLVAPEHRDAVEETTLRVRHGIRRELIPLVRVKGIGRVRARRLFNNGITGPELLAAADPSVVGHIVGGKTAESII

SEQ ID NO: 29 - protein, 775 aa, Methanothermococcus okinawensis
MLMLMEVLKENGIAELRPPQKKVVEGGLLNKNKNFLICIPTASGKTLIGEMAFINHLLDNNKTPTNKKGLFIVPLKALANEKYEEFKGKYEKYGLKIALSIGDFDEKEDLKGYDLIITTAEKLDSLIRHKVEWIKDISVVVIDEIHLIGDESRGGTLEVLLTKLKTKKTIQIIGLSATIGNPEELAKWLNAELIVDEWRPVKLKKGIGYGNKIMFIDDNGNTINEVIVDEISKNNMFNLVVDSILKDGSCIIFCNSKRGAVGEAKKLNLKKYLSPDEISELRHLKEEVLSVLDNPTKTCKDLAECIEKGVAFHHAGLTYEQRKIVEEGFRKKLIKAICCTPTLSAGINMPCRRAIIRDLKRFSSRGYIPIPKMEIHQCIGRAGRPNLDPYGEGIIYINNTENPELIENAKNYLIGNVEEIYSKLSNQKVLRTHMLGLITTGDIKNKNDLEEFIKNTFYAYQYQNTKKILENIYEITNFLEKNGFIELNYRRDENKDKSNNSHNNKKNISNTNNSIKMLVLDNNNSLTIKSRHEEDVYYNITPLGKKVSELYIDPLSAEYIIDGLKNLHKKTLSNPKNMECYILHILYIISKTTEMQPVLRVRRKEENDLINDMIKLDIDVDDVIYGISSENLEYFKNAKLFYDWINEIPEEELLLGYNIEPGILRYNVEQAKWMIHSAKEIFNLLNIDNKVIKDCLNDLEIRMEYGAKQDIIELLKIKHIGRARARILYNAGIKNANDIINNQKNIINLLGEKIARKILSELGVDTKFGQMRLSI

SEQ ID NO: 30 - protein, 8 aa, synthetic construct
  REGION 1..8: note = Exemplary Hel308 motif
QCIGRAGR

SEQ ID NO: 31 - protein, 9 aa, synthetic construct
  REGION 1..9: note = Exemplary extended Hel308 motif
QCIGRAGRP

SEQ ID NO: 32 - protein, 699 aa, Methanotorris igneus
MQKYSHVFEVLKENGIKELRPPQKKVIEKGLLNKEKNFLICIPTASGKTLIGEMALINHLLDENKTPTNKKGLFIVPLKALASEKYEEFKRKYEKYGLKVALSIGDYDEKEDLSSYNIIITTAEKLDSLMRHEIDWLNYVSVAIVDEIHMINDEKRGGTLEVLLTKLKNLDVQIIGLSATIGNPEELAEWLNAELIIDNWRPVKLRKGIFFQNKIMYLNGACKELPNFSNNPMLNLVLDCVKEGGCCLVFCNSKNGAVSEAKKLNLKKYLSNSEKYELQKLKEEILSILDPPTETCKTLAECLEKGVAFHHAGLTYEHRKIVEEGFRNKLIKVICCTPTLSAGINIPCRRAIVRDLMRFSNGRMKPIPIMEIHQCIGRAGRPGLDPYGEGIIFVKNERDLERAEQYLEGKPEYIYSKLSNQAVLRTQLLGMIATREIENEFDLISFIKNTFYAHQYGNLGGVLRNIKEVINFLEENDFIADYFPTKLGKRVSELYIDPLSAKIIIDGLKEMGNVDNEELYYLYLISKTLEMMPLLRVNSFEELDLILEMEEAGIYDRTYDDLAAFKNAKMLYDWINEVPEDEILKKYKIEPGILRYKVEQAKWMIYSTKEIAKLLNRNIDTLSKLEIRLEYGAKEDIIELLKIKYVGRARARKLYDAGIRSVEDIINNPKKVASLLGEKIAKKILGELGMKFGQQTLQI

SEQ ID NO: 33 - protein, 720 aa, Thermococcus gammatolerans
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 34 - protein, 755 aa, Thermococcus barophilus
MLSTKPKAYKRFSPIGYAMQVDELSKFGVDERIIRKIKERGISEFYPPQAEALRSGVLNGENLLLAIPTASGKTLVAEIVMLHKLFTGGGKAVYLVPLKALAEEKYREFKTWEDLGVRVAVTTGDYDSSEEWLGKYDIIIATSEKFDSLLRHKSRWIRDVTLIVADEIHLLGSYDRGATLEMILSHMLGKAQILGLSATVGNAEELAEWLNAKLVVSDWRPVKLRKGVFAHGQLIWEDGKVDKFPPQWDSLVIDAVKKGKQALVFVNTRRSAEKEAGMLGKKVRRLLTKPEARRLKELAESLESNPTNDKLKEVLVNGAAFHHAGLGRAERTLIEDAFREGLIKVLTATPTLAMGVNLPSFRVIIRDTKRYSTFGWSDIPVLEIQQMIGRAGRPKYDKEGEAIIVAKTEKPEELMEKYIFGKPEKLFSMLSNDAAFRSQVLALITNFGVESFRELIGFLEKTFYYHQRKDLEILEGKAKSIVYFLLENEFIDIDLNDSFIALPFGIRTSQLYLDPLTAKKFKDALPQIEENPNPLGIFQLLASTPDMGTLSIKRKEQESYLDYAYEMEDYLYRSIPYWEDYEFQKFLSEVKTAKLLLDWINEVSEAKLIEAYGIDTGDLYRIIELADWLMYSLIELAKVLNAGGETIKYLRRLHLRLKHGVREELLELVELPMIGRRRARALYNAGFKNVNDIVKAKPSELLAVEGIGVKVLERIYRHFGVELPLLKNIKDPDKPEDKPKEKPKPKKGTLDYFLK

SEQ ID NO: 35 - protein, 8 aa, synthetic construct
  REGION 1..8: note = Exemplary Hel308 motif
QMIGRAGR

SEQ ID NO: 36 - protein, 9 aa, synthetic construct
  REGION 1..9: note = Exemplary extended Hel308 motif
QMIGRAGRP

SEQ ID NO: 37 - protein, 744 aa, Thermococcus sibiricus
MKLNKLKSYINAFLLGMVMSMKVDELKSLGVDERILRLLRERGIEELYPPQADALKTEVLKGKNLVLAIPTASGKTLVAEIVMINKILREGGKTVYLVPLKALAEEKYKEFKFWEKLGIRIAMTTGDYDSTEEWLGKYDIIIATSEKFDSLLRHKSPWIKDINLVIADEIHLLGSYDRGATLEMILAHLDDKAQILGLSATVGNAEEVAEWLNADLVMSEWRPVALRKGVFYHGELFWEDGSIERFPTQWDSLVIDALKKGKQALVFVNTRRSAEKEALLLAGKIQRFLTKPEERKLKQLADGLDTTPTNQKLKEALTKGVAFHHAGLGRTERSIIEDAFREGLIKVITATPTLSAGVNLPAYRVIIRDTKRYSNFGWVDIPVLEIQQMMGRAGRPKYDIEGQAIIIAKTEKPEDLMKRYVLGKPEKLFSMLSNEASFRSQVLALITNFGVGNFKELVNFLERTFYYHQRKNLEALEGKAKSIVYFLFENEFIDIDLNDQFMPLPLGIRTSQLYLDPVTAKKFKDAFEKLEKNPNPLGIFQLLASTPDMSSLRVKRKEQEDLLDYAYEMEEYLYQNIPYWEDYKFEKFLGETKTAKLLLDWINEVNDVKILETYEIDTGDLYRILELVDWLMYSLIELYKLFDPKPEVLDFLKKLHIRVKHGVREELLELITLPMIGRKRARALYNAGFKGIDDIVRAKASELLKVEGIGIGVIEKIYQHFGVELPTNEKKKKVKKGTLDEFFK

SEQ ID NO: 38 - protein, 729 aa, unidentified
  source 1..729: note = Methanosarcina barkeri fusaro
MKIESLDLPDEVKQFYLNSGIMELYPPQAEAVEKGLLEGRNLLAAIPTASGKTLLAELAMLKSILAGGKALYIVPLRALASEKFRRFREFSELGIRVGISTGDYDLRDEGLGVNDIIVATSEKTDSLLRNETVWMQEISVVVADEVHLIDSPDRGPTLEVTLAKLRKMNPSCQILALSATVGNADELAVWLEAELVVSEWRPTELLEGVFFNGTFYCKDREKTVEQSTKDEAVNLALDTLKKDGQCLVFESSRKNCMAFAKKAASTVKKTLSAEDRNALAGIADEILENSETDTSTNLAVCIRSGTAFHHAGLTTPLRELVEDGFRAGRIKLISSTPTLAAGLNLPARRVIIRNYRRYSSEDGMQPIPVLEYKQMAGRAGRPRLDPYGEAVLVAKSYKEFVFLFENYIEANAEDIWSKLGTENALRTHVLSTISNGFARTYDELMDFLEATFFAFQYSNFGLSTVVNECLNFLRQEGMLEKDDALIPTSFGKLVSRLYIDPLSAARIAKGLKGAKSLSELTLLHLVCSTPDMRLLYMRSHDYQDINDYVMAHASEFVKVPSPFDTTEYEWFLGEVKTSLLLLDWIHEKSENEICLKFGTGEGDIHSIADIAEWIMHVTSQLAGLLDLKGAREAAELEKRIHYGAAPELIDLLNIRGIGRVRARKLYEAGFKSSAELAEVDPEKVAALLGPKIADRIFKQIRGRGTSSGIIASEPPEKSPYSGQKTISDY

SEQ ID NO: 39 - protein, 730 aa, Methanosarcina acetivorans
MKIESLDLPDEVKRFYENSGIPELYPPQAEAVEKGLLEGKNLLAAIPTASGKTLLAELAMLKSVLAGGKALYIVPLRALASEKFRRFQDFSELGIRVGISTGDYDRRDEGLGINDIIVATSEKTDSLLRNETAWMQEISVVVVDEVHLIDSADRGPTLEVTLAKLRKMNPFCQILALSATVGNADELAAWLDAELVLSEWRPTDLMEGVFFDGTFFCKDKEKLIEQPTKDEAINLVLDTLREGGQCLVFESSRKNCMGFAKKATSAVKKTLSAEDKEKLAGIADEILENSETDTASVLASCVRAGTAFHHAGLTSPLRELVETGFREGYVKLISSTPTLAAGLNLPARRVIIRSYRRYSSDSGMQPIPVLEYKQMAGRAGRPRLDPYGEAVLLAKSYEELLFLFEKYIEAGAEDIWSKLGTENALRTHVLSTISNGFARTKEELMDFLEATFFAYQYSNFGLSVVVDECLNFLRQEGMLEQDSDALISTMFGKLVSRLYIDPLSAALIAKGLREAGTLTELTLLHLVCSTPDMRLMYMRSQDYQDINDFVMAHAEEFSKVPSPFNIVEYEWFLSEVKTSLLLMDWIHEKPENEICLKFGTGEGDIHTTADIAEWIMHVATQLARLLDLKGAKEAAELEKRIHYGAGPELMDLLDIRGIGRVRARKLYGAGFKSTADLAGATPEKVAALVGPKIAERIFRQIGRREAVSEISDSERLEKSSQDGQSTISDF

SEQ ID NO: 40 - protein, 729 aa, Methanohalophilus mahii
MKIEELDLPSEAIEVYLQAGIEELYPPQADAVEKGLLQGENLLAAIPTASGKTLLAEMAMLKAIKKGGKALYIVPLRALASEKFRDFKRFESLGIKTAISTGDFDSRDEWLGSNDIIVATSEKTDSLLRNSTPWMKDITAVIVDEVHLLDSANRGPTLEVTLAKLKRLNPGAQVVALSATVGNAMEIAQWLEAKLVLSEWRPTYLHEGIFYGDAINFDEDQTFIERRHKEDSVNLVIDTVIQGGQCLVFDSSRRNCVGFAKKCAPAVGELLDRQNRNELEEVAKEVLENGETKLTETLAYCIKKGVAFHHAGLNSAHRRIVEDAFRNNLIKMICSTPTLAAGLNLPARRVIIRSYKRYDPNAGMQPIPVLDYKQMAGRAGRPHLDPYGEAVVIVKTYEEFTDVLERYISASAEDIWSKLGTENALRTHILSTIASGFANCHREILTFLGSTFFAHQQQSWNFEELLEDCLIFLKNEGMLEQDNETIRATELGKMISSLYIDPLSASKIIRGLEKTTHVTDMTLLQLICSTPDMRLLYLRNRDYEIINDYVMNHTEEFIEVPSPFKQIEYEWFLSEVKTALLLLEWINEKSLEKIVENYQVGEGDIYASSDIAEWLMHATQRIASRINPQLETECAKLEKRIHYGAGSELIELVEIPNVGRARARKLFKKGYRSRQKLATADEKQLAGIVGPKIAQKILSYLGRETDSNGYVEPETLENKKQQKTFQDFI

SEQ ID NO: 41 - protein, 730 aa, Methanosarcina mazei
MKIESLDLPDEIKRFYENSGILELYPPQAEAVEKGLLEGKNLLAAIPTASGKTLLAELAMLKSVLNGGKALYIVPLRALASEKFRRFQEFSVLGMRVGISTGDYDRRDEGLGINDIIVATSEKTDSLLRNETAWMQEISVVVADEVHLIDSPDRGPTLEITLSKLRRMNPSCQVLALSATVGNADELAAWLDAELVLSEWRPTDLMEGVFYNGIFYCKDKEKPVGQPTKDEAVNLVLDTIKEGGQCLVFESSRKNCMGFAKKAVSAVKKTLSNEDRETLAGIADEIIENSETDVSSVLATCVRSGTAFHHAGLTTPLRELVENGFREGRIKIISSTPTLAAGLNLPARRVIIRSYRRYSSDSGMQPIPVLEYKQMAGRAGRPRLDPYGEAVLLAKSYEEFVFLFEKYIEAGAEDIWSKLGTENALRTHILSTISNGFARTREELMDFLEATFFAFQYSNFGLSAVVDECLDFLRREGMLEKDPDALVSTVFGKLVSRLYIDPLSAALIAKGLREAGTLTELTLLHLICSTPDMRLMYMRSQDYQEVNDYVMAHAGEFSKVPNPFNIAEYEWFLGEVKTSLLLMDWIHEKPENEICLKFGIGEGDIHATADIAEWIMHVTAQLAGLLDLKGAKEASELEKRIRYGAAPELMDLLDIRSVGRVRARKLYEAGFKSTAELAAASPEHIAVLVGPKITERIFKQIGRREAVSEFSDIEPLEKGSSDGQRTISDY

SEQ ID NO: 42 - protein, 693 aa, unidentified
  source 1..693: note = Methanosaeta thermophila
MLTIRDLIRWLPESVIELYEALGIDELYPPQAEAIERGLLDGRNMIISVPTAAGKTLLAELAMLRGALSGKRSLYIVPLRALASEKFESFSRFSKLGLRVGISTGDFEKRDERLGRNDIIIATSEKADSLIRNGASWVRRIGVLVVDEIHLLDSANRGPTLEMTMTKLMHLNPEMQVIGLSATIANGREIADWIKGEIVSSDWRPVRLREGVLLEDRLVFPDGEIQLENRNRDPVLNLVLDTVDQGGQMLIFESTRRNAESMAKKVSGALQESGETIELAERLSGEGKTAKKLAMCLRHGAAFHHAGLLPEQRRLIELGFRQNVVKVIACTPTLAAGLNLPARRVLIRSYKRYEAGLGTRPIPVMEYRQMAGRAGRPGLDPYGESLIMARSESELQKLMDHYVMGEPEDIWSKLASERALRTHVLATIASRFADSVDSLSRLMASTFYARQQDPSYLGETIASVLEFLVRSDMIDKDLTPTPLGALVSRLYIDPLSAMVMIQEIRGIRRPTVLTLLHVITMTPDMELLFVQQSDNWLEDFISEHSSELGNEKNFDWLLREVKTASMLMDWINEVHEDRIEDRYSISPGDLVRIAETAEWLMSALHRISKHMDLGVTYLAERLALRIHYGAGDELLQLLELKGIGRVRARKLYQAGYRSLEDLKAADKSTLSEILGPKIAEGVISQLKEPGVSA

SEQ ID NO: 43 - protein, 739 aa, Methanosalsum zhilinae
MNINNLNLPEKVKKYYTDTGIVDLYPPQREAVDKGLLDGENIVAAIPTASGKTLLAELCMLKSIGMGGKCLYIVPLKALASEKYSRFREFESLGIKVGIATGDLDSREEWLGKNDIIIATSEKVDSLLRNESSWMKEINTVVADEVHLLNSVNRGPTLEITLAKLIHLNPGSQIIALSATIGNPEDIAGWLGARLVVSEWRPTDLYEGILLDGLLHIGNIKKDIQDESRDDAVNLVIDTVKDKGQCLVFESSRRNCMGFAKKAGKWVSKILDEHDTIQLKSLSQEIGEAGETEIADVLSRCVRQGVAFHHAGLNSEHRRMVEEGFRKNLIKMISSTPTLAAGLNLPARRVIIRSYKRYDPNFGMKPIPVLEYKQMAGRAGRPHLDPYGESVLIARSYDEFMDIMENYVNADPEDIWSKLGTENALRTHVLSTIVNGFAYTYRGLMDFVKMTFFAYQKEASDLHDVIEECVRFLIDNEMIISDSNDILPESAFRSTATGKLISMLYIDPLSGSLIMDGIRKADYFEDITMMHLICSTPDMKNLYMRSSDYENVNMYVLQNKDKFISMPSPFKMIEYEWFLGEVKTALLLLDWINEVPADDICKKYGIGEGDIRMFSETAVWLMHATSRLSGLLKVSEASEKSKELEKRLSYGINSELVNIVALKGIGRVRARKIYENGYRSIDDLKKADPLKLSKIVGSKISQKILKQLDIDVDISEIKEKDSDTVPEPESSQKTISDFT

SEQ ID NO: 44 - protein, 733 aa, Methanohalobium evestigatum
METGKLELPEYVIQFYLDTGIEKLYPPQAEAVEKGLLDNKNLLAAIPTASGKTLISELAMLKSISNGGKCLYIVPLRALASEKFERFKQFSSIGVNIGISTGDFDSTDEWLGSNDIIVATSEKADSLLRNETSWMKDITTIVVDEIHLLDSADRGPTLEITIAKLLRLNPNSQIIGLSATIGNAEEIAGWLDAELVQSQWRPIELYEGVFLEDNINFKQSQKPIKNIVKDTAVNLVLDTIDENGQCLVFESSRRNCAGFAKKAKSKVGKSLDKGLLAELNNIAEEVLETSDTETTKELASCIKRGTAFHHAGLNSAQRKIVEDNFRNNKIKVISSTPTLAAGLNLPARRVIVRNYKRYDPNFGMQPIPVLDYKQMAGRAGRPSLDPYGESVLISHTYNEFTDLLDRYIDAEPEDILSKLGTENALRTHVLSTIVNGFATTRQGMVDFMGSSFFAYQQQKWSLIDVVDDCIEFLQDNEMIKDDGERLYATRLGQVISTLYIDPLSGAIIIDKLKKADKVTDMTMLHIICSTPDMRQLYLRSKEYEKINEYVMTHSDEFVEVPNPFKSIEYEWFLGEVKTALLINEWIDEKTLDDITAEFGVGEGDINALSDISEWLMHSAVNLANLTDLDADKAQELEKRIHHGVNKDLIQLVSISNIGRVRARKLYEAGIQSVSDIKNTKLHILSNYLGRKTAYKVLEQLGVEPEDNQQIDEEPESIKSYSGNDQGQKTFNDF

SEQ ID NO: 45 - protein, 747 aa, Methanococcus maripaludis
MHVLDLLKENKITELRPPQKKVIDEGLFDKTKNFLICIPTASGKTLIGEMALLNHILDENKNLTGKKGLFIVPLKALANEKFDEFREKYEKYGIKVGLSIGDFDTKENLSKFHIIITTSEKLDSLMRHNVEWINDVSLAVIDEIHLIGDNERGGTLEVILTKLKNLNAQIVGLSATIGNPEELSNWLNAKLIVDGWRPVELKKGIYFENELEFLKNPAKKIKQVSRNNLTDLIVDSVEEKGSCLIFCNSKRNAVGEAKKHNLAKYLTRTEQHELNKLSEEILSILDRPVETCKALSKCIQNGVAFHHAGLTYKHRKIVEDGFRNRLIKVICCTPTLSAGLNLPCRRAIVRDIKRYSQNGLVDIPRMEIQQCIGRAGRPGLDPYGEGIIYIKNERDAEKAYEILTGSVENIYSKLANQKVLRIHILGLISTGEIKDGQNLVNFMKNTFYAHQFGNIGAVLLNVSEVVEFLEKNKFLETTIHKKTENKVRELSFDSSNNLVLDSKETSFDLTNPNSNIEFRSTKLGKRISELYIDPMSSEIIIEELHELKKKCDQLDRSKIDQYLFYLISKTNEMRPLLRIRPNEELDLILEMDKMGLKDYSIENIEAFKNSKMFCDWVSEIPEEIILEKYGVEPGILRYKVEQAKWMIYSTKEIAKLIHLDNSEIYKSLLKMEVRIEYGAKEELIELLNVKNVGRIRSRKLYDAGIRSKIEINKNPEKILELFGEKIGKKILGEHGMKYGQQTLLNFN

SEQ ID NO: 46 - protein, 799 aa, Natrialba magadii
MNVEELSGLPPGARSHFQEQGIEELYPPQAEAVEAGATEGENLVAAVPTASGKTMIAALSMLSAVQRGGKALYIVPLRALASEKKAEFDAYEEFGVTTGVATGNYESTSEWLATKDIIVATSEKVDSLVRNGADWLSDLTCVVSDEVHLIDDRNRGPTLEVTLAKLRRLNPQLQVVALSATVGNADELADWLDAELVDTDWRPIDLQMGVHYGNALNFDDGETREVPVEAGEKQEAALVRDILQEGGSSLVFVNSRRNAEAAARRLGQVSSRELTAGEQNDLAALATEIREDSDTETSQDLADCVERGAAFHHAGLSSTQRSLVEDAFRDRLLKVISATPTLAAGVNTPARRVIVRDWRRFDPSAGGMAPLDVLEVHQMMGRAGRPGLDPYGEAVLLAKSHDESQELFDRYVWADPEPVRSKLAAEPALRTHVLATIASGFARTREGLLEFLEATLYASQSSEGGRLERVTDDVLSYLERNDFIERSGGPEDTLNSEADAASAFTSAADLADSDGGDSGGTTGQEEDLEATSLGHTVSRLYLDPMSAAEIVHGLEDADERPTALGLYQLVSRTPDMYELYLRSGEDEKFGELYYERERELLGDAPSEFEEERFEDWLAALKTGKLLEDWATEDDEEQITERYKIGPGDLRGKVDTAEWLLGAAESLASEIDSEWAVAVREARARVEHGVGEELLELVSVSGIGRKRARRLYAAGIEEPAALRSADKGVILHVLKGEKTAENILENAGREEPSMDGVEPIPVEGGSGSGSSNSSGSSEPNADANATEDDADDNQSSLGDF

SEQ ID NO: 47 - protein, 723 aa, Methanoregula boonei
MQIQDLAIPEPLRQQYLGLGIRELYPPQAACVERGLLDGKNLLVAIPTASGKTLIAEMAMHRHIANGGKCLYIVPLKALASEKYEEFGNKGVKVGLSTGDLDRRDDALGKNDIIVATSEKVDSLLRNGARWIPDITLVVIDEIHLIDSPDRGPTLEMVIAKMRSKNPGMQLIGLSATIGNPKVLAGWLDAELVTSSWRPVDLRQGVFYDNRIQFAERMRPVKQVSKNYDDLNLCLDTIAEGGQCLVFVSSRRNAEAFAKRAAGAIKSEDAALAACAERLLEGTPTEMVKTLAACVAKGAAFHHAGLSRKERSIVEEAFRKNLLKCISSTPTLAAGLNLPARRVIIRDYLRFSAGEGMQPIPVSEYRQMAGRAGRPRLDPYGEAVLIAKEAEQVPELFEVYIEAEAEDVHSRIAEPTALYTHVLSLVASGFAGTRGELTEFMNRSFYVHEHKQGRLIHRAIDEALQFLITAEMVVEVGEHIGATELGTLVSRMYIDPRSAFAIVTTLREQEKYADLGLIQLICTTPDMPTLYAKNADLPALSRMLEVRGADIWLPPPLDDDAAETYYRAVKTAMLLSDWTDELSEEKICERYGVGPGDVFGMVENINWLLHATSQLARMFVPKFYGQIADCEICMKNGIRRELLPLVRLRGIGRVRARRLFNNGITSPEELSRHKKEDLVKILGSGIAEQVLEQLHPSKDTGKKEPPSGDKNTNPGQSTLFHFG

SEQ ID NO: 48 - protein, 681 aa, unidentified
  source 1..681: note = Ferroplasma acidarmanus
MKLSEITPSEFLKVTDNNDFTLYEHQEEAVAKLRENKNVIVSVPTASGKTLIGYISIYDTYLKGKKSMYIVPLRSLAMEKFSELLSLRNLGVKVTMSIGDYDVPPSFVKNYDVIIATSERADSMLHRDPDILNYFGLVIIDEIHMISDPSRGPRLETVISSLLYLNPEILLLGLSATVSNIQEIAEWMNAETVVSNFRAVPLETGIIFKGNLITDGEKKHLGRDDEVSLIKESIESGGQALVFRNSRRNAEKYAQSMVNFFDFQNDFEKLEIPPDLFNEAQANMVAHGVMFHHAGLSNDQRTMIEKLFKQGYIKILTATPTLAAGVNLPARTVIIRDITRFSDGYSKPISGIEIQQMIGRAGRPKYDKKGYGYIYAASPGMLRVAEGYLTGELEPVISRMDSNSLIRFNVLALISSGIATDLKGIQDFYGKTLLAAQNDIDGYELAFESALYFLKDNDFITEENDIYSATKFGRLTSDLYIDPVSSLILKKCLDLEFSEELYLYYISKTPDMLTFNYRASDYEYLEEFLDRHNISDFSEESMGAAKTAIILNEWINEVPINTIAETFGIGPGDIQAKASSADWISYSLYRLGSMFDKENENNLLHLNIRIKEGVKEEIIRIIEIPQVGRVRGRRLYNNGFKSIDDIANARVEDISRIFGFSTKLAKDIIENAGKLNNRYYR

SEQ ID NO: 49 - protein, 696 aa, Methanocaldococcus fervens
MPTNKILEILKDFGIEELRPPQKKALEKGLLDKNKNFLISIPTASGKTLIGEMALINHLLDENKNPTNKKGIFIVPLKALASEKYEEFKNKYERYGLRVALSIGDYDEDEDLSRYHLIITTAEKLDSLWRHKIDWIDDVSVVVVDEIHLINDESRGGTLEILLTKLKKFNIQIIGLSATIGNPEELANWLNAELIVDDWRPVELKKGIYKNGIIEFINGENREIKAINNNDIYNLVVDCVKDGGCCIVFCNTKRGAVNEAKKLNLKKFLTNEEKRKLKEVAEEILSILEPPTEMCKTLAECILNGSAFHHAGLTYQHRKIVEDAFRNKLIKVICCTPTLSVGLNLPCRRAIVKDLTRYTNRGMRYIPIMEIQQCIGRAGRLGLDPYGEGIIVAKNDRDYLRSYQVLTQKPEPIYSKLSNQAVLRTQLLGLIATIEIRDEYDLEWFIRNTFYAYQYGNLREVAKNINEVIRFLEEKEFMIDFIPTELGKRVAELYIDPLSAKYMIDGLNEMENEDDIYYLYLISKTLEMMPNLRVYKSEELNLIDEMENLGIKSFEIEDLEAFKTAKMLYDWISEVPEDEILKKYKIEPGILRYKVENAVWLMHALKEMAKIIGKNSEIPEKLEIRLEYGAKEDIIELLNVKYIGRVRARKLYNAGIRNVEDIINNPSKVASIIGEKITKKILEDLGIKFGQQKLIF

SEQ ID NO: 50 - protein, 729 aa, Methanocaldococcus jannaschii
MDKILEILKDFGIVELRPPQKKALERGLLDKNKNFLISIPTASGKTLIGEMALINHLLDGNKNPTNKKGIFIVPLKALASEKYEEFKSKYERYGLRIALSIGDYDEDEDLSKYHLIITTAEKLDSLWRHKIDWINDVSVVVVDEIHLINDETRGGTLEILLTKLKEFNVQIIGLSATIGNPDELAEWLNAELIVDDWRPVELKKGIYKNEAIEFINGEIREIKAVDNNDIYNLVVDCVKEGGCCLVFCNTKRNAVNEAKKLNLKKFLTEEEKIRLKEIAEEILSILEPPTEMCKTLAECILNGSAFHHAGLTYQHRKIVEDAFRKRLIKVICCTPTLSAGLNLPCRRAIVKDLTRFTNKGMRYIPIMEIQQCIGRAGRPGLDPYGEGIIVAKNDRDYLRAYQALTQKPEPIYSKLSNQAVLRTQLLGLIATGEIRDEYDLEWFIRNTFYAHQYGNLREVAKNINEVIRFLEENEFIIDFMPTELGKRVSELYIDPLSAKFIIDGLEEMENEEEIYYLYLISKTLEMMPNLRVYNSEELNLIDEMDSLGIKSFEIEDLEAFKTAKMLYDWINEVPEDEILKRYKIEPGILRYKVENAVWIMHALKEIAKLIGKSSDIPEKLEIRLEYGAKEDIIELLSIKYIGRVRARKLYNAGIRSIEDIINNPSKVASIIGEKIAKKILDELGVKFGQQKLSFSGGSAWSHPQFEKGGGSGGGSGGSAWSHPQFEKKL

SEQ ID NO: 51 - protein, 670 aa, Methanocaldococcus infernus
MDEILKFLGIKELRPPQKKALELGILDKKKNFLISIPTGAGKTVIAEMALINHLLLDKGKKGVYIVPLKALASEKYEEFKKKYEKFGVRVALSIGDYDEDEDLENYDLIITTAEKFDSLWRHGIKLSDISVVVVDEIHVIGDSERGGTLEVLLTKLKELDVQIIGLSATIGNPEELSEWLNAELLLDNWRPVELRKGIYREGVIEYLDGEVKECQDIVKEVVKDNGSVIIFCPTKKKAENRALSLDLSDLLKKSEKRKLEEISEELLSLFDPPTELCKKLASCVRKGIAFHHSGLTYEHRKIIEKAFRERILKVICSTTTLAFGLNLPCRRVIISELKRYTRRGLTYIPIMEVQQCIGRAGRPGLDEYGEGILVAKDERDYLRALQCLTQKPEPIYSKLSNDSVLRTQILGLIATRYVLDEYDLEEFIKNTFYAYQYKNLDEIKKKIKEIIEFLEDCNFIKNFEVTPLGKKVSNLYLDPLSAKIMIDNIEVKDDLHLLYILCKCIEMKPLLRVYRKEEEELAEELLNYEIFISYENLEEFKTAKMLYDWINEVPEDEILKTYKVEPGILRYKVEVAKWLSYSLKEIAKILNKEVPNLELRLEYGAKEELLELLKIKYIGRVRARKLYSAGIRNREDIIKNPKKVANILGEKISKKIFEELGVRYGQQRLI

SEQ ID NO: 52 - protein, 799 aa, Methanospirillum hungatei
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 53 - protein, 702 aa, Archaeoglobus fulgidus
MKVEELAESISSYAVGILKEEGIEELFPPQAEAVEKVFSGKNLLLAMPTAAGKTLLAEMAMVREAIKGGKSLYVVPLRALAGEKYESFKKWEKIGLRIGISTGDYESRDEHLGDCDIIVTTSEKADSLIRNRASWIKAVSCLVVDEIHLLDSEKRGATLEILVTKMRRMNKALRVIGLSATAPNVTEIAEWLDADYYVSDWRPVPLVEGVLCEGTLELFDGAFSTSRRVKFEELVEECVAENGGVLVFESTRRGAEKTAVKLSAITAKYVENEGLEKAILEENEGEMSRKLAECVRKGAAFHHAGLLNGQRRVVEDAFRRGNIKVVVATPTLAAGVNLPARRVIVRSLYRFDGYSKRIKVSEYKQMAGRAGRPGMDERGEAIIIVGKRDREIAVKRYIFGEPERITSKLGVETHLRFHSLSIICDGYAKTLEELEDFFADTFFFKQNEISLSYELERVVRQLENWGMVVEDHHLAPTKLGSLVSRLYIDPLTGFIFHDVLSRMELSDIGALHLICRTPDMERLTVRKTDSWVEEEAFRLRKELSYYPSDFSVEYDWFLSEVKTALCLKDWIEEKDEDEICAKYGIAPGDLRRIVETAEWLSNAMNRIAEEVGNTSVSGLTERIKHGVKEELLELVRIRHIGRVRARKLYNAGIRNAEDIVRHREKVASLIGRGIAERVVEGISVKSLNPESAAALEHHHHHH

SEQ ID NO: 54 - protein, 791 aa, Haloterrigena turkmenica
MNLEELTGLPPGATDHFRGEGIEELYPPQADAVEAGATDGENLVAAVPTASGKTMIAALSMLSAVQRGGKALYIVPLRALASEKKEEFEAYEEFGVTTGVTTGNYESTDDWLATKDIIVATSEKVDSLVRNGADWLSELTCVVSDEVHLIDDRNRGPTLEVTLAKLRRLNPGMQVVALSATVGNADEIADWLDASLVDTDWRPIDLQMGVHYGNALNFDDGSTREVPVEGSEKQEAALVRDILREGGSSLVFVNSRRNAEGAAKRLGQVSSREITEDERAELAELADDIRDDSDTETSADLADCVERGAAFHHAGLSSTQRSLVEDAFRDRLLKVISATPTLAAGVNTPARRVIVRDWRRFDPSAGGMAPLDVLEVHQMMGRAGRPGLDPYGEAVLLAKSHDESEELFDRYIWADPEPVRSKLAAEPALRTHVLATIASGFARTRGGLLEFLEATLYASQSSEAGRLESVTDDVLDYLERNDFIERSRDDEAEDSGEDDGPFTSAADLAEQQAAKREETLEATSLGHTVSRLYLDPMSAAEIVHGLERADERPTALGLYQLVSRTPDMYELYLRSGEDEKFGELFYERETELLGDAPSEYEEDRFEDWLAALKTGKLLEDWADETDEETITDRYKIGPGDLRGKVDTAEWLLGAAESLAAEIDSEWTVAVREARARVEHGVGEELLELVSVGGVGRKRARRLYDAGIEEPADLRSADKGIVLSVLKGEKTAENILENAGREDPSMDGVEPADGGPAVGAATNGSSGGSETDETGRADAAESDDSQSSLGDF

SEQ ID NO: 55 - protein, 752 aa, Haladaptatus paucihalophilus
MNVADLTGLPDGVPEHFHAQGIEELYPPQAEAVEAGITEGESVVASIPTASGKTFIAELAMLSSVARGGKALYIVPLRALASEKKEEFEEFEQYGVSIGVSTGNYESDGDWLASRDIIVATSEKVDSLVRNGAKWIDDLSCVVADEVHLVNDAHRGPTLEVTLAKLRRVNPDLQTVALSATVGNAGEMADWLDATLVDSTWRPIDLRKGVLYGQALHFDDGTQQELARGNEKETAALVRDTLEDGGSSLVFVNSRRNAEAAAKRLADVTKTHLTDDERRDLLDIADQIRDVSDTETSDDLATAIEKGAAFHHAGLASDHRSLVEDAFRDKLIKVISATPTLAAGVNTPSRRVIVRDWRRYDGDIGGMQPLDVLEVHQMFGRAGRPGLDPHGEAVLIAKSHDELQELFDQYVWADPEPVHSKLAAEPALRTHILATVASGFAGTEEELLDFLERTLYATQTDETGRLETVTQHVLDYLDRNGFLERDDRLRATGLGHRVSQLYLDPMSAAEIIDGLRDADGKPTALGLYHLVSRTPDMYQLYLRSGDRERYTEIAYEREPEFLGHMPSEFEDNAFEDWLSALKTARLLEDWASELDEDRITERYAIGPGDIRGKVETAQWLLNAAERLAAELQRDDAEGIPSATTTAVREARKRVEYGVEEELLDLAGVRNVGRKRARRLYEAGIESRADLREADKSVVLGALRGRKKTAENILENVGRQDPSLDDVEADAETAATSARATNDGGQQSLGDFE

SEQ ID NO: 56 - protein, 8 aa, synthetic construct
  REGION 1..8: note = Exemplary Hel308 motif
QMFGRAGR

SEQ ID NO: 57 - protein, 9 aa, synthetic construct
  REGION 1..9: note = Exemplary extended Hel308 motif
QMFGRAGRP

SEQ ID NO: 58 - protein, 783 aa, unidentified
  source 1..783: note = Halobacterium sp. NRC-1
MRVADVPGLPGGVADHFEGEGVEELYPPQAEAVERGVTEGANLVASVPTASGKTLIAQLAMLSAIAEGGDSPTFSGDGTALYIVPLRALAGEKAQEFEAFERFGLSVGVSTGNYERDGARLADNDIVVATSEKVDSLVRNGAGWIDDLSCVVADEVHLVDDDHRGPTLEVTLAKLRQQVADLQVVALSATVGNAGELAAWLDAELVDSDWRPIELRTGVHYGQSLHYDDGTQAELSVGSGSQTAAVVADTLADDGSTLVFVNSRRNAEASARRLADVTGNALSSAERERLADIAAEIRGVSDTETSDELADAVASGAAFHHAGLAREHRELVEEAFRDRLVKAVSATPTLAAGVNTPARRVVVRDWQRYDGTAGGMQPLDVLEVHQMFGRAGRPGLDPYGEAVLLANSHDELEELFDRYVYADPEPVRSKLAAEPALRTHVLAAIATGFTTTEDGLHEFLGGTLYATQTDDTGRLRSVTGDVLRYLDRNGFVERDGAALRATATGQLVSRLYVDPMSAATIIDGLRDAARDATETDDEGAFRPASELGDDAALPADASVEPTPLGLYHLVSRTPDMYELYLRSGDREQYTEVAYEHEDELLGATPREEQAEFEDWLSALKTARLMADWASELDEERIAERYDVGPGDIRGKVETAEWLLNAAERLAGELDVECGPAVREARKRVQYGVREELLGLAGVRNVGRKRARRLYNAGVESRADLRNADKGVVLGAVRGRAATAERILETVGHPDPGMDGVAADTDAAPESGGEAGGDEGQASLGDFS

SEQ ID NO: 59-62 - skipped (4 entries)

SEQ ID NO: 63 - protein, 8 aa, synthetic construct
  REGION 1..8: note = RecD motif I
  VARIANT 5: note = X = T, V or C
  VARIANT 8: note = X = T or S
GGPGXGKX

SEQ ID NO: 64 - protein, 8 aa, synthetic construct
  REGION 1..8: note = Preferred RecD motif I
GGPGTGKT

SEQ ID NO: 65 - protein, 25 aa, synthetic construct
  REGION 1..25: note = Extended RecD motif I
  VARIANT 2..17: note = X = any amino acid
  VARIANT 22: note = X = T, V or C
  VARIANT 25: note = X = T or S
QXXXXXXXXXXXXXXXXGGPGXGKX

SEQ ID NO: 66 - protein, 26 aa, synthetic construct
  REGION 1..26: note = Extended RecD motif I
  VARIANT 2..18: note = X = any amino acid
  VARIANT 23: note = X = T, V or C
  VARIANT 26: note = X = T or S
QXXXXXXXXXXXXXXXXXGGPGXGKX

SEQ ID NO: 67 - protein, 27 aa, synthetic construct
  REGION 1..27: note = Extended RecD motif I
  VARIANT 2..19: note = X = any amino acid
  VARIANT 24: note = X = T, V or C
  VARIANT 27: note = X = T or S
QXXXXXXXXXXXXXXXXXXGGPGXGKX

SEQ ID NO: 68 - skipped

SEQ ID NO: 69 - protein, 10 aa, synthetic construct
  REGION 1..10: note = RecD motif V
  VARIANT 1: note = X = Y, W or F
  VARIANT 2: note = X = A, M, C or V
  VARIANT 3: note = X = I, M or L
  VARIANT 4: note = X = T or S
  VARIANT 5: note = X = V or I
XXXXXHKSQG

SEQ ID NO: 70 - protein, 13 aa, synthetic construct
  REGION 1..13: note = MobF motif III
  VARIANT 2..3: note = X = any amino acid
  VARIANT 4: note = X = any amino acid except D, E, R and K
  VARIANT 6..10: note = X = any amino acid
  VARIANT 12: note = X = any amino acid except D, E, R and K
HXXXRXXXXXHXH

SEQ ID NO: 71 - protein, 14 aa, synthetic construct
  REGION 1..14: note = MobF motif III
  VARIANT 2..3: note = X = any amino acid
  VARIANT 4: note = X = any amino acid except D, E, R and K
  VARIANT 6..11: note = X = any amino acid
  VARIANT 13: note = X = any amino acid except D, E, R and K
HXXXRXXXXXXHXH

SEQ ID NO: 72 - protein, 15 aa, synthetic construct
  REGION 1..15: note = MobF motif III
  VARIANT 2..3: note = X = any amino acid
  VARIANT 4: note = X = any amino acid except D, E, R and K
  VARIANT 6..12: note = X = any amino acid
  VARIANT 14: note = X = any amino acid except D, E, R and K
HXXXRXXXXXXXHXH

SEQ ID NO: 73 - protein, 16 aa, synthetic construct
  REGION 1..16: note = MobF motif III
  VARIANT 2..3: note = X = any amino acid
  VARIANT 4: note = X = any amino acid except D, E, R and K
  VARIANT 6..13: note = X = any amino acid
  VARIANT 15: note = X = any amino acid except D, E, R and K
HXXXRXXXXXXXXHXH

SEQ ID NO: 74 - protein, 17 aa, synthetic construct
  REGION 1..17: note = MobF motif III
  VARIANT 2..3: note = X = any amino acid
  VARIANT 4: note = X = any amino acid except D, E, R and K
  VARIANT 6..14: note = X = any amino acid
  VARIANT 16: note = X = any amino acid except D, E, R and K
HXXXRXXXXXXXXXHXH

SEQ ID NO: 75 - protein, 18 aa, synthetic construct
  REGION 1..18: note = MobF motif III
  VARIANT 2..3: note = X = any amino acid
  VARIANT 4: note = X = any amino acid except D, E, R and K
  VARIANT 6..15: note = X = any amino acid
  VARIANT 17: note = X = any amino acid except D, E, R and K
HXXXRXXXXXXXXXXHXH

SEQ ID NO: 76 - protein, 19 aa, synthetic construct
  REGION 1..19: note = MobF motif III
  VARIANT 2..3: note = X = any amino acid
  VARIANT 4: note = X = any amino acid except D, E, R and K
  VARIANT 6..16: note = X = any amino acid
  VARIANT 18: note = X = any amino acid except D, E, R and K
HXXXRXXXXXXXXXXXHXH

SEQ ID NO: 77 - protein, 20 aa, synthetic construct
  REGION 1..20: note = MobF motif III
  VARIANT 2..3: note = X = any amino acid
  VARIANT 4: note = X = any amino acid except D, E, R and K
  VARIANT 6..17: note = X = any amino acid
  VARIANT 19: note = X = any amino acid
HXXXRXXXXXXXXXXXXHXH

SEQ ID NO: 78-84 - skipped (7 entries)

SEQ ID NO: 85 - protein, 1756 aa, Escherichia coli
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 86 - protein, 8 aa, synthetic construct
  REGION 1..8: note = RecD-like motif I of TraI Eco
GYAGVGKT

SEQ ID NO: 87 - protein, 10 aa, synthetic construct
  REGION 1..10: note = RecD-like motif V of TraI Eco
YAITAHGAQG

SEQ ID NO: 88 - protein, 14 aa, synthetic construct
  REGION 1..14: note = MobF motif III of TraI Eco
HDTSRDQEPQLHTH

SEQ ID NO: 89 - skipped

SEQ ID NO: 90 - protein, 22 aa, synthetic construct
  REGION 1..22: note = XPD motif VI
  VARIANT 2: note = Any amino acid
  VARIANT 3: note = Any amino acid except D, E, K, R. Typically G, P, A, V, L, I, M, C, F, Y, W, H, Q, N, S or T. Preferably not charged. Preferably not H. More preferably V, A, L, I or M.
  VARIANT 6: note = Any amino acid except D, E, K, R. Typically G, P, A, V, L, I, M, C, F, Y, W, H, Q, N, S or T. Preferably not charged. Preferably not H. More preferably V, A, L, I, M or C.
  VARIANT 7: note = Any amino acid except D, E, K, R. Typically G, P, A, V, L, I, M, C, F, Y, W, H, Q, N, S or T. Preferably not charged. Preferably not H. More preferably I, H, L, F, M or V.
  VARIANT 9..11: note = Any amino acid.
  VARIANT 12: note = D or E
  VARIANT 13: note = Any amino acid.
  VARIANT 14: note = Any amino acid. Preferably G, A, S or C
  VARIANT 15..16: note = Any amino acid.
  VARIANT 17: note = Any amino acid. Preferably F, V, L, I, M, A, W or Y
  VARIANT 18: note = Any amino acid. Preferably L, F, Y, M, I or V.
  VARIANT 19: note = Any amino acid. Preferably A, C, V, L, I, M or S.
QXXGRXXRXXXXXXXXXXXDNR

SEQ ID NO: 91 - protein, 726 aa, Methanococcoides burtonii
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 92 - protein, 9 aa, synthetic construct
  REGION 1..9: note = Motif V
YLWGTLSEG

SEQ ID NO: 93 - protein, 22 aa, synthetic construct
  REGION 1..22: note = Motif VI
QAMGRVVRSPTDYGARILLDGR

SEQ ID NO: 94 - protein, 65 aa, synthetic construct
  REGION 1..65: note = HhH domain
GTGSGAWKEWLERKVGEGRARRLIEYFGSAGEVGKLVENAEVSKLLEVPGIGDEAVARLVPGGSS

SEQ ID NO: 95 - protein, 299 aa, unidentified
  source 1..299: note = Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 96 - protein, 232 aa, unidentified
  source 1..232: note = Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 97 - protein, 324 aa, unidentified
  source 1..324: note = Herpes simplex virus 1
MDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKHHHHHH

SEQ ID NO: 98 - protein, 251 aa, synthetic construct
  REGION 1..251: note = PCNA subunit 1
MFKIVYPNAKDFFSFINSITNVTDSIILNFTEDGIFSRHLTEDKVLMAIMRIPKDVLSEYSIDSPTSVKLDVSSVKKILSKASSKKATIELTETDSGLKIIIRDEKSGAKSTIYIKAEKGQVEQLTEPKVNLAVNFTTDESVLNVIAADVTLVGEEMRISTEEDKIKIEAGEEGKRYVAFLMKDKPLKELSIDTSASSSYSAEMFKDAVKGLRGFSAPTMVSFGENLPMKIDVEAVSGGHMIFWIAPRLLE

SEQ ID NO: 99 - protein, 245 aa, synthetic construct
  REGION 1..245: note = PCNA subunit 2
MKAKVIDAVSFSYILRTVGDFLSEANFIVTKEGIRVSGIDPSRVVFLDIFLPSSYFEGFEVSQEKEIIGFKLEDVNDILKRVLKDDTLILSSNESKLTLTFDGEFTRSFELPLIQVESTQPPSVNLEFPFKAQLLTITFADIIDELSDLGEVLNIHSKENKLYFEVIGDLSTAKVELSTDNGTLLEASGADVSSSYGMEYVANTTKMRRASDSMELYFGSQIPLKLRFKLPQEGYGDFYIAPRAD

SEQ ID NO: 100 - protein, 246 aa, synthetic construct
  REGION 1..246: note = PCNA subunit 3
MKVVYDDVRVLKDIIQALARLVDEAVLKFKQDSVELVALDRAHISLISVNLPREMFKEYDVNDEFKFGFNTQYLMKILKVAKRKEAIEIASESPDSVIINIIGSTNREFNVRNLEVSEQEIPEINLQFDISATISSDGFKSAISEVSTVTDNVVVEGHEDRILIKAEGESEVEVEFSKDTGGLQDLEFSKESKNSYSAEYLDDVLSLTKLSDYVKISFGNQKPLQLFFNMEGGGKVTYLLAPKVLE

SEQ ID NO: 101 - protein, 608 aa, unidentified
  source 1..608: note = Bacteriophage phi-29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 102 - protein, 318 aa, unidentified
  source 1..318: note = Herpes simplex virus 1
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 103 - protein, 233 aa, unidentified
  source 1..233: note = Bacteriophage RB69
KGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAAS

SEQ ID NO: 104 - protein, 210 aa, unidentified
  source 1..210: note = Bacteriophage T7
AKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPR

SEQ ID NO: 105 - protein, 99 aa, unidentified
  source 1..99: note = Halorubrum lacusprofundi
SGEELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 106 - protein, 102 aa, Haloferax volcanii
SGEELLDLAGVRGVGRKRARRLFEAGVETRADLREADKPRVLAALRGRRKTAENILEAAGRKDPSMDAVDEDDAPDDAVPDDAGFETAKERADQQASLGDFE

SEQ ID NO: 107 - protein, 55 aa, synthetic construct
  REGION 1..55: note = (HhH)2 domain
WKEWLERKVGEGRARRLIEYFGSAGEVGKLVENAEVSKLLEVPGIGDEAVARLVP

SEQ ID NO: 108 - protein, 107 aa, synthetic construct
  REGION 1..107: note = (HhH)2-(HhH)2 domain
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 109 - protein, 7 aa, synthetic construct
  REGION 1..7: note = Peptide linker used in the Examples
SRDFWRS

SEQ ID NO: 110 - DNA, 117 bp, synthetic construct
  misc_feature 1..117: note = Polynucleotide used in the Examples
  source 1..117: mol_type = other DNA
tcgctgctccacaggtctcagcttgagcagcgaaaataagaacattatgatcagtaggagcactacgacctttgttctggtgctcgtccgggcgcccaaagtggagcgagtgccccc

SEQ ID NO: 111 - DNA, 80 bp, synthetic construct
  misc_feature 1..80: note = Polynucleotide used in the Examples
  source 1..80: mol_type = other DNA
gcactcgctccactttgggcgcccggacgagcaccagaacaaaggtcgtagtgctcctactgatcataatgttcttattt

SEQ ID NO: 112 - DNA, 33 bp, synthetic construct
  misc_feature 1..33: note = Polynucleotide used in the Examples
  source 1..33: mol_type = other DNA
tcgctgctcaagctgagacctgtggagcagcga

SEQ ID NO: 113 - DNA, 113 bp, synthetic construct
  misc_feature 1..113: note = Polynucleotide used in the Examples
  source 1..113: mol_type = other DNA
tcgctgctccacaggtctcagcttgagcagcgaaaataagaacattatgatcagtaggagcactacgacctttgttctggtgctcgtccgggcgcccaaagtggagcgagtgc

SEQ ID NO: 114 - DNA, 28 bp, synthetic construct
  misc_feature 1..28: note = Polynucleotide used in the Examples
  source 1..28: mol_type = other DNA
tcgctgctccacaggtctcagcttcccc

SEQ ID NO: 115 - DNA, 974 bp, synthetic construct
  misc_feature 1..974: note = Polynucleotide used in the Examples
  source 1..974: mol_type = other DNA
ttttttttttttttttttttttttttttttttttttttttttttttttttggttgtttctgttggtgctgatattgctgtgttctatgtcttattctgtgtatgtatcttgtctgttagccccgattgttaccggataattcgagctcggtacccaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccagtattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggatcctctagagtcgacctgcaggcatgcaagctatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattcttgaagacgaaagggcctcgtgatacgcctatttttataggttaatgtcatgataataatggtttcttagacgt

SEQ ID NO: 116 - DNA, 893 bp, synthetic construct
  misc_feature 1..893: note = Polynucleotide used in the Examples
  source 1..893: mol_type = other DNA
ctaagaaaccattattatcatgacattaacctataaaaataggcgtatcacgaggccctttcgtcttcaagaattctcatgtttgacagcttatcatcgataagctagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatagcttgcatgcctgcaggtcgactctagaggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatactggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattatccggtaacaatcggggctaacagacaagatacatacacagaataagacatagaacaca

SEQ ID NO: 117 - DNA, 28 bp, synthetic construct
  misc_feature 1..28: note = Polynucleotide used in the Examples
  source 1..28: mol_type = other DNA
gcaatatcagcaccaacagaaacaacct

SEQ ID NO: 118 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 119 - protein, 123 aa, unidentified
  source 1..123: note = Bacteriophage phi-29
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 120 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 121 - protein, 301 aa, unidentified
  source 1..301: note = Bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 122 - protein, 177 aa, synthetic construct
  REGION 1..177: note = EcoSSB-CterAla
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMAFAAAIPF

SEQ ID NO: 123 - protein, 177 aa, synthetic construct
  REGION 1..177: note = EcoSSB-CterNGGN
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMNFGGNIPF

SEQ ID NO: 124 - protein, 152 aa, synthetic construct
  REGION 1..152: note = EcoSSB-Q152del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQ

SEQ ID NO: 125 - protein, 117 aa, synthetic construct
  REGION 1..117: note = EcoSSB-G117del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQG

SEQ ID NO: 126 - protein, 970 aa, unidentified
  source 1..970: note = Citromicrobium bathyomarinum JL354
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 127 - DNA, 73 bp, synthetic construct
  misc_feature 1..73: note = Polynucleotide used in the Examples
  source 1..73: mol_type = other DNA
ttttttttttttttttttttttttttttttttttttttttttttttggttgtttctgttggtgctgatattgc

SEQ ID NO: 128 - DNA, 3523 bp, synthetic construct
  misc_feature 1..3523: note = Polynucleotide used in the Examples
  source 1..3523: mol_type = other DNA
gccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatatatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtac

SEQ ID NO: 129 - protein, 984 aa, Methanopyrus kandleri
MALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 130 - protein, 299 aa, Methanopyrus kandleri
SGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 131 - protein, 8 aa, synthetic construct
  REGION 1..8: note = RecD-like motif I of TrwC Cba
GIAGAGKS

SEQ ID NO: 132 - protein, 10 aa, synthetic construct
  REGION 1..10: note = RecD-like motif V of TrwC Cba
YALNVHMAQG

SEQ ID NO: 133 - protein, 14 aa, synthetic construct
  REGION 1..14: note = MobF motif III of TrwC Cba
HDTNRNQEPNLHFH

SEQ ID NO: 134 - protein, 943 aa, unidentified
  source 1..943: note = Halothiobacillus neapolitanus c2
MLRIKNLKGDPSAIIDYAENKKNHPDQKSGYYDAKGAPSAWGGALAADLGLSGSVQAADLKKLLSGELSDGTRFAKEDPDRRLGIDMSFSAPKSVSLAALVGGDERIIQAHDAAVRTAMSMIEQEYATARFGHAGRNVVCSGKLVYAAYRHEDARTVDDIADPQLHTHCIVSNITIDPETGKPRSIDFAWGQDGIKLAGAMYRAELARRLKEMGYELRKSEEGFELAQISDEQVETFSRRRVQVDQALEQQGTDREHASSELKTAVTLATRQGKAQLSAEDQYEEWQQRAAEAELDLSQPVGPRVSVTPPEIDLDHTFEHLSERASVINKDAVRLDALINHMSEGATLSTVDKAIQGAAVTGDVFEIEDGIKRKIITRETLKREQQILLLAQQGRGVNSVLIGVGDTKHLIEDAEQAQGFRFSEGQRRAINLTATTTDQVSGIVGAAGAGKTTAMKTVADLAKSQGLTVVGIAPSSAAADELKSAGADDTMTLATFNLKGEAAGPRLLILDEAGMVSARDGEALLKKLGKEDRLIFVGDPKQLAAVEAGSPFAQLMRSGAIQYAEITEINRQKDQKLLDIAQHFAKGKAEEAVALATKYVTEVPVTLPDKPEHKITRQAKTEARRLAIASATAKRYLELSQEERATTLVLSGTNAVRKQVNEQVRKGLIDKGEINGESFTVSTLDKADMTRAKMRKAGNYKPGQVIKTAGKQAEQSEVVAVNLDQNLIQVKLSDGTLKSIDASRFDVKKTQVFNPRQIDIAAGDKIIFTNNDQATETKNNQIGLIEEIKDGKAIINSNGAKVEIDIQRKLHIDHAYCITIHRSQGQTVDSVIVAGEASRTTTAEAAYVACTRERYKLEIITDNTERLSKNWVRYADRQTAAEALKSSEEKYPHLDEIREELRRELQQELERQEPTNITPELEIEMERSMFDQYTLHSRQPRSY

SEQ ID NO: 135 - protein, 8 aa, synthetic construct
  REGION 1..8: note = RecD-like motif I of TrwC Hne
GAAGAGKT

SEQ ID NO: 136 - protein, 10 aa, synthetic construct
  REGION 1..10: note = RecD-like motif V of TrwC Hne
YCITIHRSQG

SEQ ID NO: 137 - protein, 18 aa, synthetic construct
  REGION 1..18: note = MobF motif III of TrwC Hne
HEDARTVDDIADPQLHTH

SEQ ID NO: 138 - protein, 960 aa, unidentified
  source 1..960: note = Erythrobacter litoralis HTCC2594
MLSVANVRSPTAAASYFASDNYYASADADRSGQWIGGGAKRLGLEGKVEAKAFDALLRGELPDGSSVGNPGQAHRPGTDLSFSVPKSWSLLALVGKDERIIAAYREAVVEALQWAEKNAAETRIVEKGKMVTQATGNLAVGLFQHDTNRNQEPNLHFHAVIANVTQGKDGKWRTLKNDRLWQLNTTLNSIAMARFRVAVEKLGYEPGPVLKHGNFEARGISREQIMAFSTRRKEVLEARRGPGLEAGRIAALDTRASKEEIEDRATLGKQWSETAQSIGLDLTPLVDRARTNALGQSMEATRIGSLVERGRAWLSRFAAHVRGDPADPLVPPSVLKQDRQTIAAAQAVASAIRHLSQREAAFERTALYKAALDFGLPATIADVEKRTRALVRSGDLISGKGEHKGWLASREAVVTEQRILSEVAAGKGNSSPAIEPQKAAASVQAAAATGQGFRLNEGQLAAAELILTSKDRTIAIQGIAGAGKSSVLKPVAEVLRDEGHPVIGLAIQNTLVQMLERETGIGSQTLARFLRGWTKLLGDPGNVALRTEAQASLKDHVLVLDEASMVSNEDKEKLVRLANLAGVHRLVLIGDRKQLGAVDAGKPFALLQRAGIARAEMATNLRARDPVVREAQASAQAGDVRNALRHLKSHTVEAKGDGAQVAAETWLALDKETRARTSIYASGRAIRSAVNAAVQQGLLANREIGPGMMKLDVLDRVNATREELRHLPAYRAGQVLEISRKQQALGLSVGEYRVLGQDRKGRLVEVEDKRGKRFRFDPARIKAGKGDENLTLLEPRKLEIHEGDRIRWTRNDHRRGLFNADQARVVAIAGGKITFETSQGDQVELKRDDPMLKRIDLAYALNAHMAQGLTSDRGIAVMTSSERNLSNQKTFMVTVTRLRDHLTLVVDNAEKLGAAVARNKGEKASALEVTGSVKSTAAKGSGVDQLKPEEANKAEKELTR

SEQ ID NO: 139 - protein, 10 aa, synthetic construct
  REGION 1..10: note = RecD-like motif V of TrwC Eli
YALNAHMAQG

SEQ ID NO: 140 - protein, 853 aa, Escherichia coli
MSAIENFDAHTPMMQQYLRLKAQHPEILLFYRMGDFYELFYDDAKRASQLLDISLTKRGASAGEPIPMAGIPYHAVENYLAKLVNQGESVAICEQIGDPATSKGPVERKVVRIVTPGTISDEALLQERQDNLLAAIWQDSKGFGYATLDISSGRFRLSEPADRETMAAELQRTNPAELLYAEDFAEMSLIEGRRGLRRRPLWEFEIDTARQQLNLQFGTRDLVGFGVENAPRGLCAAGCLLQYAKDTQRTTLPHIRSITMEREQDSIIMDAATRRNLEITQNLAGGAENTLASVLDCTVTPMGSRMLKRWLHMPVRDTRVLLERQQTIGALQDFTAGLQPVLRQVGDLERILARLALRTARPRDLARMRHAFQQLPELRAQLETVDSAPVQALREKMGEFAELRDLLERAIIDTPPVLVRDGGVIASGYNEELDEWRALADGATDYLERLEVRERERTGLDTLKVGFNAVHGYYIQISRGQSHLAPINYMRRQTLKNAERYIIPELKEYEDKVLTSKGKALALEKQLYEELFDLLLPHLEALQQSASALAELDVLVNLAERAYTLNYTCPTFIDKPGIRITEGRHPVVEQVLNEPFIANPLNLSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWACAENLANKIKALTLFATHYFELTQLPEKMEGVANVHLDALEHGDTIAFMHSVQDGAASKSYGLAVAALAGVPKEVIKRARQKLRELESISPNAAATQVDGTQMSLLSVPEETSPAVEALENLDPDSLTPRQALEWIYRLKSLV

SEQ ID NO: 141 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 142 - protein, 99 aa, Sulfolobus solfataricus
EKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 143 - protein, 88 aa, Sulfolobus solfataricus
TEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 144 - protein, 107 aa, Escherichia coli
AQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 145 - protein, 237 aa, unidentified
  source 1..237: note = Bacteriophage lambda
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 146 - protein, 60 aa, unidentified
  source 1..60: note = Crenarchaea
MSSGKKPVKVKTPAGKEAELVPEKVWALAPKGRKGVKIGLFKDPETGKYFRHKLPDDYPI

SEQ ID NO: 147 - protein, 136 aa, Homo sapiens [sequence omitted]

SEQ ID NO: 148 - protein, 89 aa, unidentified
  source 1..89: note = Bacteriophage T4
MAKKEMVEFDEAIHGEDLAKFIKEASDHKLKISGYNELIKDIRIRAKDELGVDGKMFNRLLALYHKDNRDVFEAETEEVVELYDTVFSK

SEQ ID NO: 149 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 150 - protein, 375 aa, unidentified
  source 1..375: note = Citromicrobium bathyomarinum JL354
MKATIERATLLRCLSHVQSVVERRNTIPILSNVLIDADAGGGVKVMATDLDLQVVETMTAASVESAGAITVSAHLLFDIARKLPDGSQVSLETADNRMVVKAGRSRFQLPTLPRDDFPVIVEGELPTSFELPARELAEMIDRTRFAISTEETRYYLNGIFLHVSDEARPVLKAAATDGHRLARYTLDRPEGAEGMPDVIVPRKAVGELRKLLEEALDSNVQIDLSASKIRFALGGEGGVVLTSKLIDGTFPDYSRVIPTGNDKLLRLDPKAFFQGVDRVATIATEKTRAVKMGLDEDKVTLSVTSPDNGTAAEEIAAEYKAEGFEIGFNANYLKDILGQIDSDTVELHLADAGAPTLIRRDENSPALYVLMPMRV

SEQ ID NO: 151 - DNA, 88 bp, synthetic construct
  misc_feature 1..88: note = Polynucleotide used in the Examples
  source 1..88: mol_type = other DNA
cgtggtcacgaggagctcgtcctcacctcgacgtctgcacgagctttttttttttttttttttttttttttttttttttttttttttt

SEQ ID NO: 152 - DNA, 88 bp, synthetic construct
  misc_feature 1..88: note = Polynucleotide used in the Examples
  source 1..88: mol_type = other DNA
ttttttttttttttttttttttttttttttttttttttttttttgctcgtgcagacgtcgaggtgaggacgagctcctcgtgaccacg

SEQ ID NO: 153 - DNA, 44 bp, synthetic construct
  misc_feature 1..44: note = Polynucleotide used in the Examples
  source 1..44: mol_type = other DNA
cgtggtcacgaggagctcgtcctcacctcgacgtctgcacgagc

SEQ ID NO: 154 - DNA, 3560 bp, synthetic construct
  misc_feature 1..3560: note = Polynucleotide used in the Examples
  source 1..3560: mol_type = other DNA
gccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagc

SEQ ID NO: 155 - DNA, 52 bp, synthetic construct
  misc_feature 1..52: note = Polynucleotide used in the Examples
  source 1..52: mol_type = other DNA
tttttttttttttttttttttttttggttgtttctgttggtgctgatattgc

SEQ ID NO: 156 - DNA, 3556 bp, synthetic construct
  misc_feature 1..3556: note = Polynucleotide used in the Examples
  source 1..3556: mol_type = other DNA
acggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggc

(19) United States
(12) Patent Application Publication
(10) Pub. No.: US 2024/0417704 A1
(43) Pub. Date: Dec. 19, 2024

(54) MODIFIED HELICASES

(71) Applicant: Oxford Nanopore Technologies PLC, Oxford (GB)

(72) Inventors: Andrew John Heron, Oxford (GB); James Anthony Clarke, Oxford (GB); Ruth Moysey, Oxford (GB); Elizabeth Jayne Wallace, Oxford (GB); Mark John Bruce, Oxford (GB); Lakmal Nishantha Jayasinghe, Oxford (GB); Domenico Caprotti, Oxford (GB); Szabolcs Soeroes, Oxford (GB); Luke McNeill, Oxford (GB); David Antoni Alves, Oxford (GB); Rebecca Victoria Bowen, Oxford (GB); John Milton, Oxford (GB)

(73) Assignee: Oxford Nanopore Technologies PLC, Oxford (GB)

(21) Appl. No.: 18/762,324

(22) Filed: Jul. 2, 2024

Related U.S. Application Data
(63) Continuation of application No. 18/047,589, filed on Oct. 18, 2022.
Application No. 18/047,589 is a continuation of application No. 17/064,329, filed on Oct. 6, 2020, now Pat. No. 11,525,126.
Application No. 17/064,329 is a continuation of application No. 14/415,453, filed on Jan. 16, 2015, now Pat. No. 10,808,231.
(60) Provisional application No. 61/774,862, filed on Mar. 8, 2013.
(60) Provisional application No. 61/673,452, filed on Jul. 19, 2012.

(51) Int. Cl. C12N 9/14; C12N 9/90; C12Q 1/6827

(52) U.S. Cl. CPC C12N 9/14; C12N 9/90; C12Q 1/6827; C12Y 306/04012; C12Y 599/01002; C12Y 599/01003; C07K 2319/80

(57) ABSTRACT

The invention relates to modified helicases with reduced unbinding from polynucleotides. The helicases can be used to control the movement of polynucleotides and are particularly useful for sequencing polynucleotides.

### RELATED APPLICATIONS

This application is a continuation of U.S. application Ser. No. 18/047,589, filed Oct. 18, 2022, which is a continuation of U.S. application Ser. No. 17/064,329, filed Oct. 6, 2020, which is a continuation of U.S. application Ser. No. 14/415,453, filed Jan. 16, 2015, which is a national stage filing under 35 U.S.C. § 371 of International Patent Application No. PCT/GB2013/051925, filed Jul. 18, 2013, which claims the benefit of priority under 35 U.S.C. § 119(e) to U.S. Application No. 61/774,862, filed Mar. 8, 2013 and U.S. Application No. 61/673,452, filed Jul. 19, 2012, each of which is incorporated herein by reference in its entirety contents of these applications are incorporated herein by reference in their entirety.

### REFERENCE TO AN ELECTRONIC SEQUENCE LISTING

The content of the electronic sequence listing (O036670025US05-SUBSEQ-MSB.xml; Size: 238,724 bytes; and Date of Creation: Jul. 30, 2024) is herein incorporated by reference in its entirety.

### FIELD OF THE INVENTION

The invention relates to modified helicases with reduced unbinding from polynucleotides. The helicases can be used to control the movement of polynucleotides and are particularly useful for sequencing polynucleotides.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing” method, a single polynucleotide strand is passed through the pore and the identity of the nucleotides are derived. Strand sequencing can involve the use of a nucleotide handling protein, such as a helicase, to control the movement of the polynucleotide through the pore.

### SUMMARY OF THE INVENTION

Helicases are enzymes that are capable of binding to and controlling the movement of polynucleotides. Several helicases, including Hel308 helicases, have a polynucleotide binding domain which in at least one conformational state has an opening through which the polynucleotide can bind or unbind from the helicase. This allows the helicase to disengage from a polynucleotide, even if the helicase is not positioned at an end of the polynucleotide.

The inventors have surprisingly demonstrated that the ability of a helicase to control the movement of a polynucleotide can be improved by reducing the size of the opening through which the polynucleotide unbinds. In particular, the helicase's ability to control the movement of a polynucleotide can be improved by closing the opening. In accordance with the invention, the size of the opening is reduced or the opening is closed by connecting at least two parts of the helicase.

This result is surprising because a reduction in the size of the opening or a closing of the opening does not prevent the helicase from binding to a polynucleotide. Once a helicase modified in accordance with the invention has bound to a polynucleotide, it is capable of controlling the movement of most of, if not all of, the polynucleotide without unbinding or disengaging. In particular, the inventors have surprisingly demonstrated that helicases modified in accordance with the invention will strongly bind to a long polynucleotide, such as a polynucleotide comprising 400 nucleotides or more, and will control the movement of most of, if not all of, the polynucleotide. This allows the effective control of the movement of the polynucleotide, especially during Strand Sequencing.

The inventors have surprisingly demonstrated that the ability of a Hel308 helicase to control the movement of a polynucleotide can be improved by introducing one or more cysteine residues and/or one or more non-natural amino acids at specific positions. Irrespective of whether or not the introduced residues are connected, the modified Hel308 helicase is capable of controlling the movement of most of, if not all of, a polynucleotide without unbinding or disengaging.

Accordingly, the invention provides a helicase formed from one or more monomers and comprising a polynucleotide binding domain which comprises in at least one conformational state an opening through which a polynucleotide can unbind from the helicase, wherein the helicase is modified such that two or more parts on the same monomer of the helicase are connected to reduce the size of the opening and wherein the helicase retains its ability to control the movement of the polynucleotide.

The invention also provides:
a Hel308 helicase in which one or more cysteine residues and/or one or more non-natural amino acids have been introduced at one or more of the positions which correspond to D272, N273, D274, G281, E284, E285, E287, S288, T289, G290, E291, D293, T294, N300, R303, K304, N314, 5315, N316, H317, R318, K319, L320, E322, R326, N328, 5615, K717, Y720, N721 and S724 in Hel308 Mbu (SEQ ID NO: 10), wherein the helicase retains its ability to control the movement of a polynucleotide;
a construct comprising a helicase of the invention and an additional polynucleotide binding moiety, wherein the helicase is attached to the polynucleotide binding moiety and the construct has the ability to control the movement of a polynucleotide;
a method of controlling the movement of a polynucleotide, comprising contacting the polynucleotide with a helicase of the invention or a construct of the invention and thereby controlling the movement of the polynucleotide;
method of characterising a target polynucleotide, comprising (a) contacting the target polynucleotide with a transmembrane pore and a helicase of the invention or a construct of the invention such that the helicase or the construct controls the movement of the target polynucleotide through the pore and (b) taking one or more measurements as the polynucleotide moves with respect to the pore wherein the measurements are indicative of one or more characteristics of the target polynucleotide and thereby characterising the target polynucleotide;
a method of forming a sensor for characterising a target polynucleotide, comprising forming a complex between (a) a pore and (b) a helicase of the invention or a construct of the invention and thereby forming a sensor for characterising the target polynucleotide;
a sensor for characterising a target polynucleotide, comprising a complex between (a) a pore and (b) a helicase of the invention or a construct of the invention;
use of a helicase of the invention or a construct of the invention to control the movement of a target polynucleotide through a pore;
a kit for characterising a target polynucleotide comprising (a) a pore and (b) a helicase of the invention or a construct of the invention;
an apparatus for characterising target polynucleotides in a sample, comprising (a) a plurality of pores and (b) a plurality of helicases of the invention or a plurality of constructs of the invention;
a method of producing a helicase of the invention, comprising (a) providing a helicase formed from one or more monomers and comprising a polynucleotide binding domain which comprises an opening through which a polynucleotide can unbind from the helicase and (b) modifying the helicase such that two or more parts on the same monomer of the helicase are connected to reduce the size of the opening and thereby producing a helicase of the invention;
a method of producing a modified Hel308 helicase of the invention, comprising (a) providing a Hel308 helicase and (b) introducing one or more cysteine residues and/or one or more non-natural amino acids at one or more of the positions which correspond to D272, N273, D274, G281, E284, E285, E287, S288, T289, G290, E291, D293, T294, N300, R303, K304, N314, 5315, N316, H317, R318, K319, L320, E322, R326, N328, 5615, K717, Y720, N721 and S724 in Hel308 Mbu (SEQ ID NO: 10) and thereby producing a modified Hel308 helicase of the invention; and
a method of producing a construct of the invention, comprising attaching a helicase of the invention to an additional polynucleotide binding moiety and thereby producing a construct of the invention.

### DESCRIPTION OF THE FIGURES

FIG. 1 shows a coomassie stained, 7.5% Tris-HCl gel (loaded with Laemmli loading buffer) of the Hel308 Mbu(E284C/S615C)-bismaleimidePEG3 reaction mixture (SEQ ID NO: 10 with mutations E284C/S615C connected by a bismaleimidePEG3 linker). Lane X shows an appropriate protein ladder (the mass unit markers are shown on the left of the gel). Lanes a-c contain 2 μL, 5 μL or 10 μL of approximately 2.5 μM Hel308 Mbu(E284C/S615C) monomer (SEQ ID NO: 10 with mutations E284C/S615C). Lanes d-f contain 2 μL, 5 μL or 10 μL of approximately 2.5 μM Hel308 Mbu(E284C/S615C)-bismaleimidePEG3 (SEQ ID NO: 10 with mutations E284C/S615C connected by a bismaleimidePEG3 linker, i.e. a helicase in which the opening has been closed), it was clear from the gel that the reaction to attach the bismaleimidePEG3 linker went to nearly 100% yield. Arrow 1 corresponds to Hel308 Mbu (E284C/S615C) monomer (SEQ ID NO: 10 with mutations E284C/S615C) and arrow 2 corresponds to Hel308 Mbu(E284C/S615C)-bismaleimidePEG3 (SEQ ID NO: 10 with mutations E284C/S615C connected by a bismaleimidePEG3 linker, i.e. a helicase in which the opening has been closed).

FIG. 2 shows a coomassie stained 7.5% Tris-HCl gel of the Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)) reaction mixture. Lane X shows an appropriate protein ladder (the mass unit markers are shown on the left of the gel). Lane A contains 5 μL of approximately 10 μM Hel308 Mbu(E284C/S615C)-bismaleimidePEG3 (SEQ ID NO: 10 with the mutations E284C/S615C connected by a bismaleimidePEG3 linker) as a reference. The upper band (labelled 2) corresponds to Hel308 Mbu(E284C/S615C)-bismaleimidePEG3 and the lower band (labelled 1) to Hel308 Mbu (E284C/S615C) (SEQ ID NO: 10 with the mutations E284C/S615C). Lane B contains 5 μL of approximately 10 μM Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide), it was clear from the gel that the reaction to attach the mal-pep-mal linker did not go to completion as a band for the Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide) (upper band) and the Hel308 Mbu (E284C/S615C) (SEQ ID NO: 10 with the mutations E284C/S615C) (lower band) are observed. Lane C contains Hel308 Mbu (E284C/S615C) (SEQ ID NO: 10 with the mutations E284C/S615C).

FIGS. 3A-3E show a fluorescence assay used to compare the enzyme processivity of two Hel308 Mbu helicases in which the opening has been closed (Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker)) to that of the Hel308 Mbu monomer (SEQ ID NO: 10). A custom fluorescent substrate was used to assay the ability of the helicase to displace hybridised dsDNA. The fluorescent substrate (50 nM final) has a 3′ ssDNA overhang, and 80 and 33 base-pair sections of hybridised dsDNA (FIG. 3A, SEQ ID NO: 110, labelled 1). The major bottom “template” strand is hybridised to an 80 nt “blocker” strand (SEQ ID NO: 111, labelled 2), adjacent to its 3′ overhang, and a 33 nt fluorescent probe (labelled 3), labelled at its 5′ and 3′ ends with carboxyfluorescein (FAM, labelled 4) and black-hole quencher (BHQ-1, labelled 5) bases (SEQ ID NO: 112), respectively. When hybridised, the FAM is distant from the BHQ-1 and the substrate is essentially fluorescent. In the presence of ATP (1 mM) and MgCl₂ (10 mM), the helicase (labelled 6, 10 nM) binds to the substrate's 3′ overhang (SEQ ID NO: 110), moves along the lower strand, and begins to displace the 80 nt blocker strand (SEQ ID NO: 111), as shown in FIG. 3B. If processive, the helicase displaces the fluorescent probe too (FIG. 3C, SEQ ID NO: 112, labelled with a carboxyfluorescein (FAM) at its 5′ end a black-hole quencher (BHQ-1) at its 3′ end). The fluorescent probe is designed in such a way that its 5′ and 3′ ends are self-complementary and thus form a kinetically-stable hairpin once displaced, preventing the probe from re-annealing to the template strand (FIG. 3D). Upon formation of the hairpin product, the FAM is brought into the vicinity of the BHQ-1 and its fluorescence is quenched. A processive enzyme, capable of displacing the 80 mer “blocker” (SEQ ID NO: 111) and fluorescent (SEQ ID NO: 112, labelled with a carboxyfluorescein (FAM) at its 5′ end a black-hole quencher (BHQ-1) at its 3′ end) strands will therefore lead to a decrease in fluorescence over time. However, if the enzyme has a processivity of less than 80 nt it would be unable to displace the fluorescent strand (SEQ ID NO: 112, labelled with a carboxyfluorescein (FAM) at its 5′ end a black-hole quencher (BHQ-1) at its 3′ end) and, therefore, the “blocker” strand (SEQ ID NO: 111) would reanneal to the major bottom strand (FIG. 3E).

FIGS. 4A-4B show additional custom fluorescent substrates which were also used for control purposes. The substrate used as a negative control was identical to that of the one described in FIGS. 3A-3E but lacking the 3′overhang (FIG. 4A, (SEQ ID NO's: 111 (labelled 2 in figure), 112 (Strand labelled 3 in figure, labelled with a carboxyfluorescein (FAM, labelled 4 in figure) at its 5′ end a black-hole quencher (BHQ-1, labelled 5 in figure) at its 3′ end) and 113 labelled 7 in figure)). A similar substrate to that described in FIGS. 3A-3E but lacking the 80 base pair section (SEQ ID NO's: 112 (strand labelled 3 in figure, labelled with a carboxyfluorescein (FAM labelled 4 in figure) at its 5′ end a black-hole quencher (BHQ-1, labelled 5 in figure) at its 3′ end) and 114 labelled 8 in figure), was used as a positive control for active, but not necessarily processive, helicases (FIG. 4B).

FIG. 5 shows a graph (y-axis label=Normalised fluorescence (arbitrary values), x-axis label=time (min)) of the time-dependent fluorescence changes upon testing Hel308 Mbu, Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker) against the processivity substrate shown in FIGS. 3A-3E in buffered solution (400 mM NaCl, 10 mM Hepes pH 8.0, 1 mM ATP, 10 mM MgCl₂, 50 nM fluorescent substrate DNA (SEQ ID NOs: 110, 111 and 112 (labelled with a carboxyfluorescein (FAM) at its 5′ end a black-hole quencher (BHQ-1) at its 3′ end). The data points marked with a black diamond correspond to a buffer blank, the white square data points correspond to Hel308 Mbu monomer (SEQ ID NO: 10), the black cross data points correspond to Hel308 Mbu(E284C/S615C)-bismaleimidePEG11 (SEQ ID NO: 10 with mutations E284C/S615C connected by a bismaleimidePEG11 linker) and the white circle data points correspond to Hel308 Mbu(E284C/S615C)-bismaleimidePEG3 (SEQ ID NO: 10 with mutations E284C/S615C connected by a bismaleimidePEG3 linker). The decrease in fluorescence exhibited by Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker), denote the increased processivity of these complexes as compared to Hel308 Mbu monomer (SEQ ID NO: 10).

FIG. 6 shows a graph (y-axis label=Normalised fluorescence (arbitrary values), x-axis label=time (min)) of the time-dependent fluorescence changes upon testing Hel308 Mbu (SEQ ID NO: 10), Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker) against the positive control processivity substrate (shown in FIG. 4B, SEQ ID NOs: 112 (labelled with a carboxyfluorescein (FAM) at its 5′ end a black-hole quencher (BHQ-1) at its 3′ end) and 60) in buffered solution (400 mM NaCl, 10 mM Hepes pH 8.0, 1 mM ATP, 10 mM MgCl₂, 50 nM fluorescent substrate DNA (SEQ ID NOs: 112 (labelled with a carboxyfluorescein (FAM) at its 5′ end a black-hole quencher (BHQ-1) at its 3′ end) and 114)). The data points marked with a black diamond correspond to a buffer blank, the white square data points correspond to Hel308 Mbu monomer (SEQ ID NO: 10), the black cross data points correspond to Hel308 Mbu(E284C/S615C)-bismaleimidePEG11 (SEQ ID NO: 10 with mutations E284C/S615C connected by a bismaleimidePEG11 linker) and the white circle data points correspond to Hel308 Mbu(E284C/S615C)-bismaleimidePEG3 (SEQ ID NO: 10 with mutations E284C/S615C connected by a bismaleimidePEG3 linker). This positive control demonstrated that all complexes were indeed active, as denoted by a fluorescence decrease for all samples.

FIG. 7 shows a schematic of enzyme controlled translocation of a polynucleotide through a nanopore in a membrane, where the enzyme controls the movement of the polynucleotide against the force of the applied field. The schematic shows the example of a 3′ to 5′ enzyme (labelled A), where the capture of a polynucleotide in the pore by the 5′ end leads to the enzyme controlling the movement of the polynucleotide (the polynucleotide sequences used in example 4 are SEQ ID NO: 115 (labelled B in FIG. 7), SEQ ID NO:116 (labelled C in FIG. 7) and SEQ ID NO: 117 (labelled D in FIG. 7)) against the force of the applied field. During DNA capture the hybridised strands are unzipped. Arrow 1 denotes the direction of DNA movement through the nanopore, the white arrow 2 denotes the direction of enzyme movement along the DNA and arrow 3 denotes the direction of the applied field. As long as the enzyme does not dissociate from the DNA the enzyme will pull the DNA out of the pore until it is finally ejected on the cis side of the membrane.

FIG. 8 shows a schematic of enzyme controlled translocation of a polynucleotide through a nanopore in a membrane, where the enzyme controls the movement of the polynucleotide in the same direction as the force of the applied field. The schematic shows the example of a 3′ to 5′ enzyme (labelled A), where the capture of a polynucleotide in the pore by the 3′ end leads to the enzyme controlling the movement of the polynucleotide with the force of the applied field. Arrow 1 denotes the direction of the DNA movement through the nanopore, the white arrow 2 denotes the direction of enzyme movement along DNA and arrow 3 denotes the direction of the applied field. As long as the enzyme does not dissociate from the DNA the enzyme will feed the DNA through the pore until it is finally ejected on the trans side of the membrane.

FIG. 9 shows the DNA substrate design used in Example 4. The 900mer sense strand (SEQ ID NO: 115) is labelled A, the anti-sense strand which is minus the 4 base-pair leader (SEQ ID NO: 116) is labelled B and the primer (SEQ ID NO: 117) is labelled C. The primer has a 3′ cholesterol tag which is labelled D.

FIG. 10 shows example current traces observed when a helicase controls the translocation of DNA (+140 mV, 400 mM NaCl, 100 mM Hepes pH 8.0, 10 mM potassium ferrocyanide, 10 mM potassium ferricyanide, 0.1 nM 900mer DNA (SEQ ID NO: 115, 116 and 117 (which at the 3′ end of the sequence has six iSp18 spacers attached to two thymine residues and a 3′ cholesterol TEG)), 1 mM ATP, 1 mM MgCl₂) through an MspA nanopore (MS(B1-G75S/G77S/L88N/Q126R)8 MspA (SEQ ID NO: 2 with mutations G75S/G77S/L88N/Q126R) using Hel308 Mbu monomer (200 nM, SEQ ID NO: 10). The top electrical trace (y-axis label=current (pA), x-axis label=time (min)) shows the open pore current (˜120 pA) dropping to a DNA level (20-50 pA) when DNA is captured under the force of the applied potential (+140 mV). DNA with enzyme attached results in a long block that shows stepwise changes in current as the enzyme moves the DNA through the pore. The upper trace shows a sequence of 8 separate helicase-controlled DNA movements marked A-H. All of the helicase-controlled DNA movements in this section of trace are being moved through the nanopore against the field by the enzyme (DNA captured 5′down) (see FIG. 7 for details). Below are enlargements of the last section of 4 of the helicase-controlled DNA movements as the DNA exits the nanopore. Of the 8 helicase-controlled DNA movements in this section, only 1 (H) ends in the characteristic long polyT level that indicates that the enzyme has reached the end of the DNA and moved the 50T 5′-leader of the DNA substrate through the pore (labelled with a *). In the full run with Hel308Mbu (SEQ ID NO: 10) it was found that ˜30% of the helicase-controlled DNA movements end at the polyT (n=19 helicase-controlled DNA movements in this experiment).

FIG. 11 shows example current traces observed when a helicase controls the translocation of DNA (+140 mV, 400 mM NaCl, 100 mM Hepes pH 8.0, 10 mM potassium ferrocyanide, 10 mM potassium ferricyanide, 0.05 nM 900mer DNA (SEQ ID NO: 115, 116 and 117 (which at the 3′ end of the sequence has six iSp18 spacers attached to two thymine residues and a 3′ cholesterol TEG)), 2 mM ATP, 2 mM MgCl₂) through an MspA nanopore (MS(B1-G75S/G77S/L88N/Q126R)8 MspA (SEQ ID NO: 2 with mutations G75S/G77S/L88N/Q126R) using the Hel308 Mbu(E284C/S615C)-bismaleimidePEG3 (10 nM, SEQ ID NO: 10 with mutations E284C/S615C connected by a bismaleimidePEG3 linker). The top electrical trace (y-axis label=current (pA), x-axis label=time (min)) shows the open pore current (˜115 pA) dropping to a DNA level (15-40 pA) when DNA is captured under the force of the applied potential (+140 mV). DNA with enzyme attached results in a long block that shows stepwise changes in current as the enzyme moves the DNA through the pore. The upper trace shows a sequence of 8 separate helicase-controlled DNA movements marked A-H. All the helicase-controlled DNA movements in this section of trace are being moved through the nanopore against the field by the enzyme (DNA captured 5′down) (see FIG. 7 for details). Below are enlargements of the last section of 4 of the helicase-controlled DNA movements as the DNA exits the nanopore. Of the 8 helicase-controlled DNA movements in this section, every one ends in the characteristic long polyT level that indicates that the enzyme has reached the end of the DNA and moved the 50T 5′-leader of the DNA substrate through the pore (labelled with a *). In the full run with Hel308 Mbu (E284C/S615C)-bismaleimidePEG3 (SEQ ID NO: 10 with mutations E284C/S615C connected by a bismaleimidePEG3 linker) it was found that ˜85% of the helicase-controlled DNA movements against the field (5′ down) end at the polyT (n=27 helicase-controlled DNA movements in this experiment).

FIG. 12 shows example current traces observed when a helicase controls the translocation of DNA (+140 mV, 400 mM NaCl, 100 mM Hepes pH 8.0, 10 mM potassium ferrocyanide, 10 mM potassium ferricyanide, 0.05 nM 900mer DNA (SEQ ID NO: 115, 116 and 117 (which at the 3′ end of the sequence has six iSp18 spacers attached to two thymine residues and a 3′ cholesterol TEG)), 2 mM ATP, 2 mM MgCl₂) through an MspA nanopore (MS(B1-G75S/G77S/L88N/Q126R)8 MspA (SEQ ID NO: 2 with mutations G75S/G77S/L88N/Q126R) using the Hel308 Mbu(E284C/S615C)-bismaleimidePEG3 (10 nM, SEQ ID NO: 10 with mutations E284C/S615C connected by a bismaleimidePEG3 linker). The top electrical trace (y-axis label=current (pA), x-axis label=time (min)) shows the open pore current (˜120 pA) dropping to a DNA level (15-40 pA) when DNA is captured under the force of the applied potential (+140 mV). DNA with enzyme attached results in a long block that shows stepwise changes in current as the enzyme moves the DNA through the pore. The upper trace shows a sequence of 4 separate helicase-controlled DNA movements marked A-D. All the helicase-controlled DNA movements in this section of trace are being moved through the nanopore with the field by the enzyme (DNA captured 3′down) (see FIG. 8 for details). Below are enlargements of the last section of the helicase-controlled DNA movements as the DNA exits the nanopore. 3′ down DNA shows a characteristically different signature to 5′ down DNA, with a different current to sequence relationship, and different variance. Of the 4 helicase-controlled DNA movements in this section, every one ends in the characteristic long polyT level that indicates that the enzyme has reached the end of the DNA and moved the 50T 5′-leader of the DNA substrate through the pore (labelled with a *). In the full run with Hel308 Mbu(E284C/S615C)-bismaleimidePEG3 (SEQ ID NO: 10 with mutations E284C/S615C connected by a bismaleimidePEG3 linker) it was found that ˜87% of the helicase-controlled DNA movements with the field (3′ down) end at the polyT (n=15 helicase-controlled DNA movements in this experiment).

FIG. 13 shows example current traces (y-axis=current (pA), x-axis=time (s) for upper and lower traces) observed when a helicase controls the translocation of DNA (+120 mV, (625 mM KCl, 100 mM Hepes, 75 mM Potassium Ferrocyanide (II), 25 mM Potassium ferricyanide (III), pH 8, 0.5 nM DNA (SEQ ID NO: 127 attached at its 3′ end to four iSpC3 spacers, the last of which is attached to the 5′ end of SEQ ID NO: 128), 1 mM ATP, 10 mM MgCl₂) through an MspA nanopore (MS(B1-G75S/G77S/L88N/Q126R)8 MspA (SEQ ID NO: 2 with mutations G75S/G77S/L88N/Q126R) using the Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)). The top electrical trace shows the open pore current (˜400 pA) dropping to a DNA level (250-220 pA) when DNA is captured under the force of the applied potential (+120 mV). DNA with enzyme attached results in a long block that shows stepwise changes in current as the enzyme moves the DNA through the pore. The lower trace is a zoomed in region of the upper trace.

FIG. 14 shows a coomassie stained 7.5% Tris-HCl gel of the TrwC Cba-N691C/Q346C-mal-PEG11-mal (SEQ ID NO: 126 with the mutations N691C/Q346C connected by a bismaleimide polyethylene glycol linker) reaction mixture. The lane on the right of the gel (labelled M) shows an appropriate protein ladder (the mass unit markers are shown on the right of the gel). Lane 1 contains 5 μL of approximately 10 μM TrwC Cba-D657C/R339C alone (SEQ ID NO: 126 with mutation D657C/R339C) as a reference. Lane 2 contains 5 μL of approximately 10 μM TrwC Cba-N691C/Q346C-bismaleimidePEG11 (SEQ ID NO: 126 with the mutations N691C/Q346C connected by a bismaleimide PEG11 linker). As indicated in lane 2, the upper band corresponds to the dimeric enzyme species (labelled A), the middle band corresponds to the closed complex (labelled B) TraI-Cba-N691C/Q346C-bidmaleimidePEG11 (SEQ ID NO: 126 with the mutations N691C/Q346C connected by a bismaleimide PEG11 linker). It was clear from the gel that the reaction to attach the mal-PEG11-mal linker did not go to completion as a band for unmodified starting material (labelled C) TrwC Cba-N691C/Q346C (SEQ ID NO: 126 with the mutations N691C/Q346C) was observed.

FIG. 15 shows a fluorescence assay for testing the rate of turnover of dsDNA molecules (min⁻¹enzyme⁻¹). A custom fluorescent substrate was used to assay the ability of the helicase (a) to displace hybridised dsDNA. 1) The fluorescent substrate strand (50 nM final, SEQ ID NO: 151 and 152) has both a 3′ and 5′ ssDNA overhang. The upper strand (b) has a carboxyfluorescein base (c) near the 5′ end (the carboxyfluorescein is attached to a modified thymine at position 6 in SEQ ID NO: 151), and the hybridised complement (d) has a black-hole quencher (BHQ-1) base (e) near the 3′ end (the black-hole quencher is attached to a modified thymine at position 81 in SEQ ID NO: 152). When hybridised, the fluorescence from the fluorescein is quenched by the local BHQ-1, and the substrate is essentially non-fluorescent. 1 μM of a capture strand (f, SEQ ID NO: 153) that is part-complementary to the lower strand of the fluorescent substrate is included in the assay. 2) In the presence of ATP (1 mM) and MgCl₂ (10 mM), helicase (10 nM) added to the substrate binds to the 3′ tail of the fluorescent substrate, moves along the upper strand, and displaces the complementary strand (d) as shown. 3) Once the complementary strand with BHQ-1 is fully displaced the fluorescein on the major strand fluoresces. 4) Displaced lower strand (d) preferentially anneals to an excess of capture strand (f) to prevent re-annealing of initial substrate and loss of fluorescence.

FIG. 16 shows dsDNA turnover (enzyme⁻¹min⁻¹) in buffer (400 mM KCl, 100 mM Hepes pH 8.0, 1 mM ATP, 10 mM MgCl₂, 50 nM fluorescent substrate DNA (SEQ ID NOs: 151 and 152), 1 μM capture DNA (SEQ ID NO: 153)) for a number of helicases (Hel308 Mbu (labelled 1, SEQ ID NO: 10), Hel308 Mbu-E284C (labelled 2, SEQ ID NO: 10 with the mutation E284C), Hel308 Mbu-E284C/C301A (labelled 3, SEQ ID NO: 10 with the mutations E284C/C301A), Hel308 Mbu-E285C (labelled 4, SEQ ID NO: 10 with the mutation E285C) and Hel308 Mbu-S288C (labelled 5, SEQ ID NO: 10 with the mutation S288C)). Hel308 Mbu-E284C (SEQ ID NO: 10 with the mutation E284C), Hel308 Mbu-E284C/C301A (SEQ ID NO: 10 with the mutations E284C/C301A), Hel308 Mbu-E285C (SEQ ID NO: 10 with the mutation E285C) and Hel308 Mbu-S288C (SEQ ID NO: 10 with the mutation S288C) showed increased rate of turnover of dsDNA molecules (min⁻¹enzyme⁻¹) when compared to Hel308 Mbu (SEQ ID NO: 10).

FIG. 17 shows dsDNA turnover (enzyme⁻¹min⁻¹) in buffer (400 mM KCl, 100 mM Hepes pH 8.0, 1 mM ATP, 10 mM MgCl₂, 50 nM fluorescent substrate DNA (SEQ ID NOs: 151 and 152), 1 μM capture DNA (SEQ ID NO: 153)) for a number of helicases (Hel308 Mbu (labelled 1, SEQ ID NO: 10), Hel308 Mbu-E284C (labelled 2, SEQ ID NO: 10 with the mutation E284C) and Hel308 Mbu-D274C (labelled 6, SEQ ID NO: 10 with the mutation D274C)). Hel308 Mbu-E284C (SEQ ID NO: 10 with the mutation E284C) and Hel308 Mbu-D274C (SEQ ID NO: 10 with the mutations D274C) showed increased rate of turnover of dsDNA molecules (min⁻¹enzyme⁻¹) when compared to Hel308 Mbu (SEQ ID NO: 10).

FIG. 18 shows a schematic of enzyme controlled translocation of a polynucleotide through a nanopore in a membrane, where the enzyme controls the movement of the polynucleotide against the force of the applied field. The schematic shows the example of a 3′ to 5′ enzyme (labelled A), where the capture of a polynucleotide (the polynucleotide sequences used in Example 9 are SEQ ID NO: 154 (labelled B in FIG. 18), SEQ ID NO: 155 (labelled C in FIG. 18) and SEQ ID NO: 156 (labelled D in FIG. 18) and SEQ ID NO: 117 (labelled E in FIG. 18)) in the pore by the 5′ end leads to the enzyme controlling the movement of the polynucleotide against the force of the applied field (the direction of the applied field is indicated by arrow 1). During DNA capture the hybridised strands are unzipped. Arrow 2 denotes the direction of DNA movement through the nanopore and the arrow 3 denotes the direction of enzyme movement along the DNA. As long as the enzyme does not dissociate from the DNA the enzyme will pull the DNA out of the pore until it is finally ejected on the cis side of the membrane.

FIG. 19 shows an example current trace (y-axis=current (pA), x-axis=time (s)) observed when Hel308 Mbu (SEQ ID NO: 10) controls the translocation of DNA (+120 mV, (960 mM KCl, 25 mM potassium phosphate, 3 mM potassium ferrocyanide, 1 mM potassium ferricyanide pH 8.0, 10 mM MgCl₂ and 1 mM ATP) 0.2 nM DNA ((SEQ ID NO: 154 attached at its 5′ end to four nitroindoles the last of which is attached to the 3′ end of SEQ ID NO: 155), SEQ ID NO: 156 and SEQ ID NO: 117) through an MspA nanopore (MS(B1-G75S/G77S/L88N/Q126R)8 MspA (SEQ ID NO: 2 with mutations G75S/G77S/L88N/Q126R). The electrical trace shows the open pore current (˜250 pA) dropping to a DNA level (˜50 pA) when DNA is captured under the force of the applied potential (+120 mV). DNA with enzyme attached results in a long block that shows stepwise changes in current as the enzyme moves the DNA through the pore.

FIG. 20 shows example current traces (y-axis=current (pA), x-axis=time (s) for both traces) observed when Hel308 Mbu-E284C (SEQ ID NO: 10 with the mutation E284C) controls the translocation of DNA (+120 mV, (960 mM KCl, 25 mM potassium phosphate, 3 mM potassium ferrocyanide, 1 mM potassium ferricyanide pH 8.0, 10 mM MgCl₂ and 1 mM ATP) 0.2 nM DNA ((SEQ ID NO: 154 attached at its 5′ end to four nitroindoles the last of which is attached to the 3′ end of SEQ ID NO: 155), SEQ ID NO: 156 and SEQ ID NO: 117) through an MspA nanopore (MS(B1-G75S/G77S/L88N/Q126R)8 MspA (SEQ ID NO: 2 with mutations G75S/G77S/L88N/Q126R). The upper electrical trace shows the DNA level (˜35 pA) when DNA is captured under the force of the applied potential (+120 mV). DNA with enzyme attached results in a long block that shows stepwise changes in current as the enzyme moves the DNA through the pore. The lower trace shows a zoomed in view of the helicase controlled DNA movement shown in the upper trace.

FIG. 21 shows example current traces (y-axis=current (pA), x-axis=time (s) for both traces) observed when Hel308 Mbu-S288C (SEQ ID NO: 10 with the mutation S288C) controls the translocation of DNA (+120 mV, (960 mM KCl, 25 mM potassium phosphate, 3 mM potassium ferrocyanide, 1 mM potassium ferricyanide pH 8.0, 10 mM MgCl₂ and 1 mM ATP) 0.2 nM DNA ((SEQ ID NO: 154 attached at its 5′ end to four nitroindoles the last of which is attached to the 3′ end of SEQ ID NO: 155), SEQ ID NO: 156 and SEQ ID NO: 117) through an MspA nanopore (MS(B1-G75S/G77S/L88N/Q126R)8 MspA (SEQ ID NO: 2 with mutations G75S/G77S/L88N/Q126R). The upper electrical trace shows the DNA level (˜40 pA) when DNA is captured under the force of the applied potential (+120 mV). DNA with enzyme attached results in a long block that shows stepwise changes in current as the enzyme moves the DNA through the pore. The lower trace shows a zoomed in view of the helicase controlled DNA movement shown in the upper trace.

FIG. 22 shows example current traces (y-axis=current (pA), x-axis=time (s) for both traces) observed when Hel308 Mbu-E284Faz (SEQ ID NO: 10 with the mutation E284Faz) controls the translocation of DNA (+120 mV, (960 mM KCl, 25 mM potassium phosphate, 3 mM potassium ferrocyanide, 1 mM potassium ferricyanide pH 8.0, 10 mM MgCl₂ and 1 mM ATP) 0.2 nM DNA ((SEQ ID NO: 154 attached at its 5′ end to four nitroindoles the last of which is attached to the 3′ end of SEQ ID NO: 155), SEQ ID NO: 156 and SEQ ID NO: 117) through an MspA nanopore (MS(B1-G75S/G77S/L88N/Q126R)8 MspA (SEQ ID NO: 2 with mutations G75S/G77S/L88N/Q126R). The upper electrical trace shows the DNA level (˜50 pA) when DNA is captured under the force of the applied potential (+120 mV). DNA with enzyme attached results in a long block that shows stepwise changes in current as the enzyme moves the DNA through the pore. The lower trace shows a zoomed in view of the helicase controlled DNA movement shown in the upper trace.

FIG. 23 shows example current traces (y-axis=current (pA), x-axis=time (s) for both traces) observed when heat treated Hel308 Mbu-E284Faz (SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block) controls the translocation of DNA (+120 mV, (960 mM KCl, 25 mM potassium phosphate, 3 mM potassium ferrocyanide, 1 mM potassium ferricyanide pH 8.0, 10 mM MgCl₂ and 1 mM ATP) 0.2 nM DNA ((SEQ ID NO: 154 attached at its 5′ end to four nitroindoles the last of which is attached to the 3′ end of SEQ ID NO: 155), SEQ ID NO: 156 and SEQ ID NO: 117) through an MspA nanopore (MS(B1-G75S/G77S/L88N/Q126R)8 MspA (SEQ ID NO: 2 with mutations G75S/G77S/L88N/Q126R). The upper electrical trace shows the DNA level (˜50 pA) for a number of helicase controlled DNA movements (each movement is numbered 1-3) when DNA is captured under the force of the applied potential (+120 mV). DNA with enzyme attached results in a long block that shows stepwise changes in current as the enzyme moves the DNA through the pore. The lower trace shows a zoomed in view of the helicase controlled DNA movement labelled 1 in the upper trace.

### DESCRIPTION OF THE SEQUENCE LISTING

SEQ ID NO: 1 shows the codon optimised polynucleotide sequence encoding the MS-B1 mutant MspA monomer. This mutant lacks the signal sequence and includes the following mutations: D90N, D91N, D93N, D118R, D134R and E139K.

SEQ ID NO: 2 shows the amino acid sequence of the mature form of the MS-B1 mutant of the MspA monomer. This mutant lacks the signal sequence and includes the following mutations: D90N, D91N, D93N, D118R, D134R and E139K.

SEQ ID NO: 3 shows the polynucleotide sequence encoding one monomer of ca-hemolysin-E111N/K147N (α-HL-NN; Stoddart et al., PNAS, 2009; 106(19): 7702-7707).

SEQ ID NO: 4 shows the amino acid sequence of one monomer of α-HL-NN.

SEQ ID NOs: 5 to 7 show the amino acid sequences of MspB, C and D.

SEQ ID NO: 8 shows the amino acid sequence of the Hel308 motif.

SEQ ID NO: 9 shows the amino acid sequence of the extended Hel308 motif.

SEQ ID NOs: 10 to 58 show the amino acid sequences of Hel308 helicases in Table 1.

SEQ ID NO: 59 shows the RecD-like motif I.

SEQ ID NOs: 60 to 62 show the extended RecD-like motif I.

SEQ ID NO: 63 shows the RecD motif I.

SEQ ID NO: 64 shows a preferred RecD motif I, namely G-G-P-G-T-G-K-T.

SEQ ID NOs: 65 to 67 show the extended RecD motif I.

SEQ ID NO: 68 shows the RecD-like motif V.

SEQ ID NO: 69 shows the RecD motif V.

SEQ ID NOs: 70 to 77 show the MobF motif III.

SEQ ID NOs: 78 to 84 show the MobQ motif III.

SEQ ID NO: 85 shows the amino acid sequence of TraI Eco.

SEQ ID NO: 86 shows the RecD-like motif I of TraI Eco.

SEQ ID NO: 87 shows the RecD-like motif V of TraI Eco.

SEQ ID NO: 88 shows the MobF motif III of TraI Eco.

SEQ ID NO: 89 shows the XPD motif V.

SEQ ID NO: 90 shows XPD motif VI.

SEQ ID NO: 91 shows the amino acid sequence of XPD Mbu.

SEQ ID NO: 92 shows the XPD motif V of XPD Mbu.

SEQ ID NO: 93 shows XPD motif V I of XPD Mbu.

SEQ ID NO: 94 shows the amino acid sequence of a preferred HhH domain.

SEQ ID NO: 95 shows the amino acid sequence of the ssb from the bacteriophage RB69, which is encoded by the gp32 gene.

SEQ ID NO: 96 shows the amino acid sequence of the ssb from the bacteriophage T7, which is encoded by the gp2.5 gene.

SEQ ID NO: 97 shows the amino acid sequence of the UL42 processivity factor from Herpes virus 1.

SEQ ID NO: 98 shows the amino acid sequence of subunit 1 of PCNA.

SEQ ID NO: 99 shows the amino acid sequence of subunit 2 of PCNA.

SEQ ID NO: 100 shows the amino acid sequence of subunit 3 of PCNA.

SEQ ID NO: 101 shows the amino acid sequence of Phi29 DNA polymerase.

SEQ ID NO: 102 shows the amino acid sequence (from 1 to 319) of the UL42 processivity factor from the Herpes virus 1.

SEQ ID NO: 103 shows the amino acid sequence of the ssb from the bacteriophage RB69, i.e. SEQ ID NO: 95, with its C terminus deleted (gp32RB69CD).

SEQ ID NO: 104 shows the amino acid sequence (from 1 to 210) of the ssb from the bacteriophage T7 (gp2.5T7-R211Del). The full length protein is shown in SEQ ID NO: 96.

SEQ ID NO: 105 shows the amino acid sequence of the 5^{th} domain of Hel308 Hla.

SEQ ID NO: 106 shows the amino acid sequence of the 5^{th} domain of Hel308 Hvo.

SEQ ID NO: 107 shows the amino acid sequence of the (HhH)2 domain.

SEQ ID NO: 108 shows the amino acid sequence of the (HhH)2-(HhH)2 domain.

SEQ ID NO: 109 shows the amino acid sequence of the peptide linker used to form a helicase in which the opening has been closed.

SEQ ID NOs: 110 to 117 show polynucleotide sequences used in the Examples.

SEQ ID NO: 118 shows the amino acid sequence of the human mitochondrial SSB (HsmtSSB).

SEQ ID NO: 119 shows the amino acid sequence of the p5 protein from Phi29 DNA polymerase.

SEQ ID NO: 120 shows the amino acid sequence of the wild-type SSB from E. coli.

SEQ ID NO: 121 shows the amino acid sequence of the ssb from the bacteriophage T4, which is encoded by the gp32 gene.

SEQ ID NO: 122 shows the amino acid sequence of EcoSSB-CterAla.

SEQ ID NO: 123 shows the amino acid sequence of EcoSSB-CterNGGN.

SEQ ID NO: 124 shows the amino acid sequence of EcoSSB-Q152del.

SEQ ID NO: 125 shows the amino acid sequence of EcoSSB-G117del.

SEQ ID NO: 126 shows the amino acid sequence of TrwC Cba.

SEQ ID NO: 127 shows part of the polynucleotide sequence used in Example 5. Attached to the 3′ end of this sequence are four iSpC3 spacers units the last of which is attached to the 5′ end of SEQ ID NO: 128.

SEQ ID NO: 128 shows part of the polynucleotide sequence used in Example 5. Attached to the 5′ end of this sequence are four iSpC3 spacers units the last of which is attached to the 3′ end of SEQ ID NO: 127.

SEQ ID NO: 129 shows the amino acid sequence of Topoisomerase V Mka (Methanopyrus Kandleri).

SEQ ID NO: 130 shows the amino acid sequence of domains H-L of Topoisomerase V Mka (Methanopyrus Kandleri).

SEQ ID NOs: 131 to 139 show some of the TraI sequences shown in Table 3.

SEQ ID NO: 140 shows the amino acid sequence of Mutant S (Escherichia coli).

SEQ ID NO: 141 shows the amino acid sequence of Sso7d (Sufolobus solfataricus).

SEQ ID NO: 142 shows the amino acid sequence of Sso10b1 (Sulfolobus solfataricus P2).

SEQ ID NO: 143 shows the amino acid sequence of Sso10b2 (Sulfolobus solfataricus P2).

SEQ ID NO: 144 shows the amino acid sequence of Tryptophan repressor (Escherichia coli).

SEQ ID NO: 145 shows the amino acid sequence of Lambda repressor (Enterobacteria phage lambda).

SEQ ID NO: 146 shows the amino acid sequence of Cren7 (Histone crenarchaea Cren7 Sso).

SEQ ID NO: 147 shows the amino acid sequence of human histone (Homo sapiens).

SEQ ID NO: 148 shows the amino acid sequence of dsbA (Enterobacteria phage T4).

SEQ ID NO: 149 shows the amino acid sequence of Rad51 (Homo sapiens).

SEQ ID NO: 150 shows the amino acid sequence of PCNA sliding clamp (Citromicrobium bathyomarinum JL354).

SEQ ID NO: 151 shows one of the sequences used in Example 7. This sequence has a carboxyfluorescein attached to a modified thymine located at position 6.

SEQ ID NO: 152 shows one of the sequences used in Example 7. This sequence has a black-hole quencher (BHQ-1) attached to a modified thymine at position 81.

SEQ ID NO: 153 shows one of the sequences used in Example 7.

SEQ ID NO: 154 shows one of the sequences used in Example 9. This sequence is attached at its 5′ end by four nitroindoles to the 3′ end of SEQ ID NO: 155.

SEQ ID NO: 155 shows one of the sequences used in Example 9. This sequence is attached at its 3′ end by four nitroindoles to the 5′ end of SEQ ID NO: 154.

SEQ ID NO: 156 shows one of the sequences used in Example 9.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a helicase” includes “helicases”, reference to “an opening” includes two or more such openings, reference to “a transmembrane protein pore” includes two or more such pores, and the like.

All publications, patents and patent applications cited herein, whether supra or infra, are hereby incorporated by reference in their entirety.

Modified Helicases with Two or More Parts Connected

The present invention provides a modified helicase that is useful for controlling the movement of a polynucleotide. The modified helicase is based on an unmodified helicase having one or more monomers. In other words, the helicase may be monomeric or oligomeric/multimeric. This is discussed in more detail below. The modified helicase is based on an unmodified helicase comprising a polynucleotide binding domain which comprises in at least one conformational state an opening through which a polynucleotide can unbind from the helicase. In accordance with the invention, the helicase is modified such that two or more parts on the same monomer of the helicase are connected to reduce the size of the opening. The reduced size of the opening does not prevent the helicase from binding to a polynucleotide. For instance, the helicase may bind to a polynucleotide at one of its termini. The reduced size of the opening decreases the ability of the polynucleotide to unbind or disengage from the helicase, particularly from internal nucleotides of the polynucleotide. This is discussed in more detail below and allows the modified helicase to remain bound to the polynucleotide for longer. The modified helicase has the ability to control the movement of a polynucleotide. The modified helicase is artificial or non-natural.

The ability of a helicase to bind to and unbind from a polynucleotide can be determined using any method known in the art. Suitable binding/unbinding assays include, but are not limited to, native polyacrylamide gel electrophoresis (PAGE), fluorescence anisotropy, calorimetry and Surface plasmon resonance (SPR, such as Biacore™). The ability of a helicase to unbind from a polynucleotide can of course be determined by measuring the time for which the helicase can control the movement of a polynucleotide. This may also be determined using any method known in the art. The ability of a helicase to control the movement of a polynucleotide is typically assayed in a nanopore system, such as the ones described below. The ability of a helicase to control the movement of a polynucleotide can be determined as described in the Examples.

A modified helicase of the invention is a useful tool for controlling the movement of a polynucleotide during Strand Sequencing. A problem which occurs in sequencing polynucleotides, particularly those of 500 nucleotides or more, is that the molecular motor which is controlling the movement of the polynucleotide may disengage from the polynucleotide. This allows the polynucleotide to be pulled through the pore rapidly and in an uncontrolled manner in the direction of the applied field. A modified helicase of the invention is less likely to unbind or disengage from the polynucleotide being sequenced. The modified helicase can provide increased read lengths of the polynucleotide as they control the movement of the polynucleotide through a nanopore. The ability to move an entire polynucleotide through a nanopore under the control of a modified helicase of the invention allows characteristics of the polynucleotide, such as its sequence, to be estimated with improved accuracy and speed over known methods. This becomes more important as strand lengths increase and molecular motors are required with improved processivity. A modified helicase of the invention is particularly effective in controlling the movement of target polynucleotides of 500 nucleotides or more, for example 1000 nucleotides, 5000, 10000, 20000, 50000, 100000 or more.

A modified helicase of the invention is also a useful tool for isothermal polymerase chain reaction (PCR). In such methods, the strands of double stranded DNA are typically first separated by a helicase of the invention and coated by single stranded DNA (ssDNA)-binding proteins. In the second step, two sequence specific primers typically hybridise to each border of the DNA template. DNA polymerases may then be used to extend the primers annealed to the templates to produce a double stranded DNA and the two newly synthesized DNA products may then be used as substrates by the helicases of the invention, entering the next round of the reaction. Thus, a simultaneous chain reaction develops, resulting in exponential amplification of the selected target sequence.

The modified helicase has the ability to control the movement of a polynucleotide. The ability of a helicase to control the movement of a polynucleotide can be assayed using any method known in the art. For instance, the helicase may be contacted with a polynucleotide and the position of the polynucleotide may be determined using standard methods. The ability of a modified helicase to control the movement of a polynucleotide is typically assayed in a nanopore system, such as the ones described below and, in particular, as described in the Examples.

A modified helicase of the invention may be isolated, substantially isolated, purified or substantially purified. A helicase is isolated or purified if it is completely free of any other components, such as lipids, polynucleotides, pore monomers or other proteins. A helicase is substantially isolated if it is mixed with carriers or diluents which will not interfere with its intended use. For instance, a helicase is substantially isolated or substantially purified if it is present in a form that comprises less than 10%, less than 5%, less than 2% or less than 1% of other components, such as lipids, polynucleotides, pore monomers or other proteins.

A helicase for use in the invention comprises a polynucleotide binding domain. A polynucleotide binding domain is the part of the helicase that is capable of binding to a polynucleotide. Polynucleotides are defined below. The ability of a domain to bind a polynucleotide can be determined using any method known in the art. The polynucleotide binding domains of known helicases have typically been identified in the art. The domain (with or without bound polynucleotide) may be identified using protein modelling, x-ray diffraction measurement of the protein in a crystalline state (Rupp B (2009). Biomolecular Crystallography: Principles, Practice and Application to Structural Biology. New York: Garland Science.), nuclear magnetic resonance (NMR) spectroscopy of the protein in solution (Mark Rance; Cavanagh, John; Wayne J. Fairbrother; Arthur W. Hunt III; Skelton, NNicholas J. (2007). Protein NMR spectroscopy: principles and practice (2nd ed.). Boston: Academic Press.) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33: 307-69. Structural information of proteins determined by above mentioned methods are publicly available from the protein bank (PDB) database.

Protein modelling exploits the fact that protein structures are more conserved than protein sequences amongst homologues. Hence, producing atomic resolution models of proteins is dependent upon the identification of one or more protein structures that are likely to resemble the structure of the query sequence. In order to assess whether a suitable protein structure exists to use as a “template” to build a protein model, a search is performed on the protein data bank (PDB) database. A protein structure is considered a suitable template if it shares a reasonable level of sequence identity with the query sequence. If such a template exists, then the template sequence is “aligned” with the query sequence, i.e. residues in the query sequence are mapped onto the template residues. The sequence alignment and template structure are then used to produce a structural model of the query sequence. Hence, the quality of a protein model is dependent upon the quality of the sequence alignment and the template structure.

Proteins, such as helicases, are dynamic structures which are in constant motion. The conformational space that a protein can explore has been described by an energy landscape, in which different conformations are populated based on their energies, and rates of interconversion are dependent on the energy barriers between states (Vinson, Science, 2009: 324(5924): 197). Helicases can therefore exist in several conformation states whether in isolation or controlling the movement of a polynucleotide. In at least one conformational state, the polynucleotide binding domain of an unmodified helicase for use in the invention comprises an opening through which a polynucleotide can unbind from the helicase. The opening may be present in all conformational states of the helicase, but does not have to be. For instance, in all conformational states, the polynucleotide binding domain may comprise an opening through which a polynucleotide can unbind from the helicase. Alternatively, in one or more conformational states of the helicase, the polynucleotide binding domain may comprise an opening through which a polynucleotide cannot unbind from the helicase because the opening is too small. In one or more conformational states of the helicase, the polynucleotide binding domain may not comprise an opening through which a polynucleotide can unbind from the helicase.

The polynucleotide binding domain preferably comprises in at least one conformational state an opening through which one or more internal nucleotides of the polynucleotide can unbind from the helicase. An internal nucleotide is a nucleotide which is not a terminal nucleotide in the polynucleotide. For example, it is not a 3′ terminal nucleotide or a 5′ terminal nucleotide. All nucleotides in a circular polynucleotide are internal nucleotides. Reducing or preventing the unbinding from one or more internal nucleotides in accordance with the invention is advantageous because it results in modified helicases that are capable of binding to one terminus of a polynucleotide, controlling the movement of most, if not all of, the polynucleotide and then unbinding at the other terminus. Such helicases are particularly helpful for Strand Sequencing.

The ability of one or more internal nucleotide to unbind from the helicase may be determined by carrying out a comparative assay. For instance, the ability of a helicase to unbind from a control polynucleotide A is compared with its ability to unbind from the same polynucleotide but with a blocking group attached at the terminal nucleotides (polynucleotide B). The blocking group prevents any unbinding at the terminal nucleotide of strand B, and thus allows only internal unbinding of the helicase. Alternatively, the ability of a helicase to unbind from a circular polynucleotide may be assayed. Unbinding may be assayed as described above.

The opening may be a groove, pocket or recess in the polynucleotide binding domain.

The presence of an opening through which a polynucleotide can unbind from the helicase can be determined using any method known in the art. The presence of an opening can be determined by measuring the ability of a helicase to unbind from a polynucleotide, and in particular from internal nucleotides of the polynucleotide, as discussed in more detail above. Openings in the polynucleotide domain can be identified using protein modelling, x-ray diffraction, NMR spectroscopy or cryo-electron microscopy as discussed above.

In accordance with the invention, the helicase is modified by connecting two or more parts on the same monomer of the helicase. If the helicase is oligomeric or multimeric, the two or more parts cannot be on different monomers. Any number of parts, such as 3, 4, 5 or more parts, may be connected. Preferred methods of connecting the two or more parts are discussed in more detail below.

The two or more parts can be located anywhere on the monomer as long as they reduce the size of the opening when connected in accordance with the invention. The two or more parts may be in the polynucleotide domain or the opening, but do not have to be. For instance, one, both or all of the two or more parts may be outside the polynucleotide binding domain, such as on different domain of the helicase. The maximum distance between the two or more parts is the circumference of the helicase.

The two or more parts are preferably spatially proximate. The two or more parts are preferably less that 50 Angstroms (Å) apart, such as less than 40 Å apart, less than 30 Å apart, less than 25 Å apart, less than 20 Å apart, less than 10 Å apart or less than 10 Å apart.

At least one of the two or more parts preferably forms part of the opening, is adjacent to the opening or is near the opening. It is straightforward to identify parts of the opening, such as amino acids within the opening, as described above. Parts are adjacent to the opening if they are next to, but do not form part of the opening. For instance, an amino acid which is located next to an amino acid that forms part of the opening, but which itself does not form part of the opening is adjacent to the opening. In the context of the invention, “next to” may mean next to in the amino acid sequence of the helicase or next two in the three-dimensional structure of the helicase. A part is typically near to the opening if it is less than 20 Å from an amino acid that forms part of the opening, such as less than 15 Å, less than 10 Å, less than 5 Å or less than 2Å apart from an amino acid that forms part of the opening. A part is typically near to the opening if it is within 1, 2, 3, 4 or 5 amino acids of an amino acid that forms part of the opening in the amino acid sequence of the helicase. Such amino acids may be identified as discussed above.

The two or more parts may be on opposite sides of the opening. The two or more parts may be on the same side of the opening. In this embodiment, the two or more parts of the helicase may be connected to form a loop, lid, constriction or flap that reduces the size of the opening.

The two or more parts are preferably on the surface of the monomer, i.e. on the surface of the helicase. It is straightforward to connect two or more parts on the surface as described in more detail below. Surface parts may be determined using protein modelling, x-ray diffraction, NMR spectroscopy or cryo-electron microscopy as discussed above.

The modified helicase retains its ability to control the movement of a polynucleotide. This ability of the helicase is typically provided by its three dimensional structure that is typically provided by its β-strands and α-helices. The α-helices and β-strands are typically connected by loop regions. In order to avoid affecting the ability of the helicase to control the movement of a polynucleotide, the two or more parts are preferably loop regions of the monomer. The loop regions of specific helicases can be identified using methods known in the art, such as protein modelling, x-ray diffraction, NMR spectroscopy or cryo-electron microscopy as discussed above.

For Hel308 helicases (SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58), β-strands can only be found in the two RecA-like engine domains (domains 1 and 2). These domains are responsible for coupling the hydrolysis of the fuel nucleotide (normally ATP) with movement. The important domains for ratcheting along a polynucleotide are domains 3 and 4, but above all domain 4. Interestingly, both of domains 3 and 4 comprise only α-helices. There is an important α-helix in domain 4 called the ratchet helix. As a result, in the Hel308 embodiments of the invention, the two or more parts are preferably not in any of the α-helixes.

The size of the opening may be reduced to any degree as long as it reduces the unbinding of the polynucleotides from the helicase. This may be determined as discussed above. Ways in which the size of the opening are reduced are discussed in more detail below.

The two or more parts are preferably connected to close the opening. If the opening is closed, the polynucleotide cannot unbind from the helicase through the opening. The helicase is more preferably modified such that it does not comprise the opening in any conformational state. If the opening is not present in any conformational state of the helicase, the polynucleotide cannot unbind from the helicase through the opening. The helicase is most preferably modified such that it is capable of forming a covalently-closed structure around the polynucleotide. Once the covalently-closed structure is bound to a polynucleotide, for instance at one end of the polynucleotide, it is capable of controlling the movement of the polynucleotide without unbinding until it reaches the other end.

### Connection

The two or more parts may be connected in any way. The connection can be transient, for example non-covalent. Even transient connection will reduce the size of the opening and reduce unbinding of the polynucleotide from the helicase through the opening.

The two or more parts are preferably connected by affinity molecules. Suitable affinity molecules are known in the art. The affinity molecules are preferably (a) complementary polynucleotides (International Application No. PCT/GB10/000132 (published as WO 2010/086602), (b) an antibody or a fragment thereof and the complementary epitope (Biochemistry 6th Ed, W.H. Freeman and co (2007) pp 953-954), (c) peptide zippers (O'Shea et al., Science 254 (5031): 539-544), (d) capable of interacting by β-sheet augmentation (Remaut and Waksman Trends Biochem. Sci. (2006) 31 436-444), (e) capable of hydrogen bonding, pi-stacking or forming a salt bridge, (f) rotaxanes (Xiang Ma and He Tian Chem. Soc. Rev., 2010, 39, 70-80), (g) an aptamer and the complementary protein (James, W. in Encyclopedia of Analytical Chemistry, R. A. Meyers (Ed.) pp. 4848-4871 John Wiley & Sons Ltd, Chichester, 2000) or (h) half-chelators (Hammerstein et al. J Biol Chem. 2011 Apr. 22; 286(16): 14324-14334). For (e), hydrogen bonding occurs between a proton bound to an electronegative atom and another electronegative atom. Pi-stacking requires two aromatic rings that can stack together where the planes of the rings are parallel. Salt bridges are between groups that can delocalize their electrons over several atoms, e.g. between aspartate and arginine.

The two or more parts may be transiently connected by a hexa-his tag or Ni-NTA. The two or more parts may also be modified such that they transiently connect to each other.

The two or more parts are preferably permanently connected. In the context of the invention, a connection is permanent if is not broken while the helicase is used or cannot be broken without intervention on the part of the user, such as using reduction to open —S—S— bonds.

The two or more parts are preferably covalently-attached. The two or more parts may be covalently attached using any method known in the art.

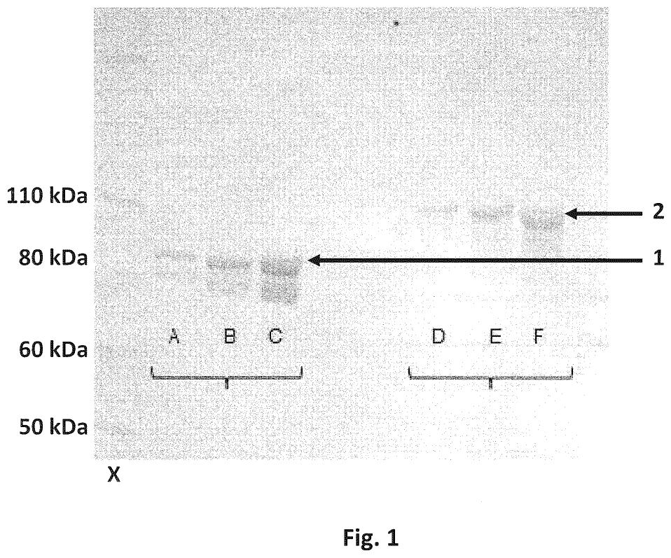

The two or more parts may be covalently attached via their naturally occurring amino acids, such as cysteines, threonines, serines, aspartates, asparagines, glutamates and glutamines. Naturally occurring amino acids may be modified to facilitate attachment. For instance, the naturally occurring amino acids may be modified by acylation, phosphorylation, glycosylation or farnesylation. Other suitable modifications are known in the art. Modifications to naturally occurring amino acids may be post-translation modifications. The two or more parts may be attached via amino acids that have been introduced into their sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), any one of the amino acids numbered 1-71 included in FIG. 1 of Liu C. C. and Schultz P. G., Annu. Rev. Biochem., 2010, 79, 413-444 or any one of the amino acids listed below. The introduced amino acids may be modified as discussed above.

In a preferred embodiment, the two or more parts are connected using linkers. Linker molecules are discussed in more detail below. One suitable method of connection is cysteine linkage. This is discussed in more detail below. The two or more parts are preferably connected using one or more, such as two or three, linkers. The one or more linkers may be designed to reduce the size of, or close, the opening as discussed above. If one or more linkers are being used to close the opening as discussed above, at least a part of the one or more linkers is preferably oriented such that it is not parallel to the polynucleotide when it is bound by the helicase. More preferably, all of the linkers are oriented in this manner. If one or more linkers are being used to close the opening as discussed above, at least a part of the one or more linkers preferably crosses the opening in an orientation that is not parallel to the polynucleotide when it bound by the helicase. More preferably, all of the linkers cross the opening in this manner. In these embodiments, at least a part of the one or more linkers may be perpendicular to the polynucleotide. Such orientations effectively close the opening such that the polynucleotide cannot unbind from the helicase through the opening.

Each linker may have two or more functional ends, such as two, three or four functional ends. Suitable configurations of ends in linkers are well known in the art.

One or more ends of the one or more linkers are preferably covalently attached to the helicase. If one end is covalently attached, the one or more linkers may transiently connect the two or more parts as discussed above. If both or all ends are covalently attached, the one or more linkers permanently connect the two or more parts.

At least one of the two or more parts is preferably modified to facilitate the attachment of the one or more linkers. Any modification may be made. The linkers may be attached to one or more reactive cysteine residues, reactive lysine residues or non-natural amino acids in the two or more parts. The non-natural amino acid may be any of those discussed above. The non-natural amino acid is preferably 4-azido-L-phenylalanine (Faz). At least one amino acid in the two or more parts is preferably substituted with cysteine or a non-natural amino acid, such as Faz.

The one or more linkers are preferably amino acid sequences and/or chemical crosslinkers.

Suitable amino acid linkers, such as peptide linkers, are known in the art. The length, flexibility and hydrophilicity of the amino acid or peptide linker are typically designed such that it reduces the size of the opening, but does not to disturb the functions of the helicase. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline. The amino acid sequence of a linker preferably comprises a polynucleotide binding moiety. Such moieties and the advantages associated with their use are discussed below.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to dithiothreitol (DTT).

Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyl]amine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S-S-PEG3-biotin, DBCO-S-S-PEG3-biotin, DBCO-S-S-PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). The most preferred crosslinker is maleimide-propyl-SRDFWRS-(1,2-diaminoethane)-propyl-maleimide as used in the Examples.

The one or more linkers may be cleavable. This is discussed in more detail below.

The two or more parts may be connected using two different linkers that are specific for each other. One of the linkers is attached to one part and the other is attached to another part. The linkers should react to form a modified helicase of the invention. The two or more parts may be connected using the hybridization linkers described in International Application No. PCT/GB10/000132 (published as WO 2010/086602). In particular, the two or more parts may be connected using two or more linkers each comprising a hybridizable region and a group capable of forming a covalent bond. The hybridizable regions in the linkers hybridize and link the two or more parts. The linked parts are then coupled via the formation of covalent bonds between the groups. Any of the specific linkers disclosed in International Application No. PCT/GB10/000132 (published as WO 2010/086602) may be used in accordance with the invention.

The two or more parts may be modified and then attached using a chemical crosslinker that is specific for the two modifications. Any of the crosslinkers discussed above may be used.

The linkers may be labeled. Suitable labels include, but are not limited to, fluorescent molecules (such as Cy3 or AlexaFluor®555), radioisotopes, e.g. ¹²⁵I, ³⁵S, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting the two or more parts is via cysteine linkage. This can be mediated by a bi-functional chemical crosslinker or by an amino acid linker with a terminal presented cysteine residue. Linkage can occur via natural cysteines in the helicase. Alternatively, cysteines can be introduced into the two or more parts of the helicase. If the two or more parts are connected via cysteine linkage, the one or more cysteines have preferably been introduced to the two or more parts by substitution.

The length, reactivity, specificity, rigidity and solubility of any bi-functional linker may be designed to ensure that the size of the opening is reduced sufficiently and the function of the helicase is retained. Suitable linkers include bismaleimide crosslinkers, such as 1,4-bis(maleimido)butane (BMB) or bis(maleimido)hexane. One draw back of bi-functional linkers is the requirement of the helicase to contain no further surface accessible cysteine residues if attachment at specific sites is preferred, as binding of the bi-functional linker to surface accessible cysteine residues may be difficult to control and may affect substrate binding or activity. If the helicase does contain several accessible cysteine residues, modification of the helicase may be required to remove them while ensuring the modifications do not affect the folding or activity of the helicase. This is discussed in International Application No. PCT/GB10/000133 (published as WO 2010/086603). The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S⁻ group. The reactivity of cysteine residues may be protected by thiol protective groups such as 5,5′-dithiobis-(2-nitrobenzoic acid) (dTNB). These may be reacted with one or more cysteine residues of the helicase before a linker is attached. Selective deprotection of surface accessible cysteines may be possible using reducing reagents immobilized on beads (for example immobilized tris(2-carboxyethyl) phosphine, TCEP). Cysteine linkage of the two or more parts is discussed in more detail below.

Another preferred method of attaching the two or more parts is via 4-azido-L-phenylalanine (Faz) linkage. This can be mediated by a bi-functional chemical linker or by a polypeptide linker with a terminal presented Faz residue. The one or more Faz residues have preferably been introduced to the helicase by substitution. Faz linkage of two or more helicases is discussed in more detail below.

### Helicase

Any helicase formed of one or monomers and comprising a polynucleotide binding domain which comprises in at least one conformational state an opening through which a polynucleotide can unbind from the helicase may modified in accordance with the invention. Helicases are often known as translocases and the two terms may be used interchangeably.

Suitable helicases are well-known in the art (M. E. Fairman-Williams et al., Curr. Opin. Struct Biol., 2010, 20 (3), 313-324, T. M. Lohman et al., Nature Reviews Molecular Cell Biology, 2008, 9, 391-401).

The helicase is preferably a member of superfamily 1 or superfamily 2. The helicase is more preferably a member of one of the following families: Pif1-like, Upf1-like, UvrD/Rep, Ski-like, Rad3/XPD, NS3/NPH-II, DEAD, DEAH/RHA, RecG-like, REcQ-like, T1R-like, Swi/Snf-like and Rig-I-like. The first three of those families are in superfamily 1 and the second ten families are in superfamily 2. The helicase is more preferably a member of one of the following subfamilies: RecD, Upf1 (RNA), PcrA, Rep, UvrD, Hel308, Mtr4 (RNA), XPD, NS3 (RNA), Mss116 (RNA), Prp43 (RNA), RecG, RecQ, T1R, RapA and Hef (RNA). The first five of those subfamilies are in superfamily 1 and the second eleven subfamilies are in superfamily 2. Members of the Upf1, Mtr4, NS3, Mss116, Prp43 and Hef subfamilies are RNA helicases. Members of the remaining subfamilies are DNA helicases.

The helicase may be a multimeric or oligomeric helicase. In other words, the helicase may need to form a multimer or an oligomer, such as a dimer, to function. In such embodiments, the two or more parts cannot be on different monomers. The helicase is preferably monomeric. In other words, the helicase preferably does not need to form a multimer or an oligomer, such as a dimer, to function. Hel308, RecD, TraI and XPD helicases are all monomeric helicases. These are discussed in more detail below. Methods for determining whether or not a helicase is oligomeric/multimeric or monomeric are known in the art. For instance, the kinetics of radiolabelled or fluorescently-labelled polynucleotide unwinding using the helicase can be examined. Alternatively, the helicase can be analysed using size exclusion chromatography.

Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. The two or more parts may be present on the same or different domains of a monomeric helicase. The unmodified helicase suitable for modification in accordance with the invention is preferably capable of binding to the target polynucleotide at an internal nucleotide. Internal nucleotides are defined above.

Generally, a helicase which is capable of binding at an internal nucleotide is also capable of binding at a terminal nucleotide, but the tendency for some helicases to bind at an internal nucleotide will be greater than others. For an unmodified helicase suitable for modification in accordance with the invention, typically at least 10% of its binding to a polynucleotide will be at an internal nucleotide. Typically, at least 20%, at least 30%, at least 40% or at least 50% of its binding will be at an internal nucleotide. Binding at a terminal nucleotide may involve binding to both a terminal nucleotide and adjacent nucleotides at the same time. For the purposes of the invention, this is not binding to the target polynucleotide at an internal nucleotide. In other words, the helicase for modification using the invention is not only capable of binding to a terminal nucleotide in combination with one or more adjacent internal nucleotides. The helicase may be capable of binding to an internal nucleotide without concurrent binding to a terminal nucleotide.

A helicase which is capable of binding at an internal nucleotide may bind to more than one internal nucleotide. Typically, the helicase binds to at least 2 internal nucleotides, for example at least 3, at least 4, at least 5, at least 10 or at least 15 internal nucleotides. Typically the helicase binds to at least 2 adjacent internal nucleotides, for example at least 3, at least 4, at least 5, at least 10 or at least 15 adjacent internal nucleotides. The at least 2 internal nucleotides may be adjacent or non-adjacent.

If modification in accordance with the invention closes the opening such that unbinding from internal nucleotides is prevented, it is preferred that the unmodified helicase is capable of at least some binding to a terminal nucleotide. This will allow the modified helicase to bind to a polynucleotide at one terminus and control the movement of the polynucleotide along its entire length without unbinding. The helicase will eventually unbind from the polynucleotide at the opposite terminus from which it became bound.

The ability of a helicase to bind to a polynucleotide at an internal nucleotide may be determined by carrying out a comparative assay. The ability of a helicase to bind to a control polynucleotide A is compared to the ability to bind to the same polynucleotide but with a blocking group attached at the terminal nucleotide (polynucleotide B). The blocking group prevents any binding at the terminal nucleotide of strand B, and thus allows only internal binding of a helicase. Alternatively, the ability of a helicase to bind to an internal nucleotide may also be assayed using circular polynucleotides.

Examples of helicases which are capable of binding at an internal nucleotide include, but are not limited to, Hel308 Tga, Hel308 Mhu and Hel308 Csy. Hence, the helicase preferably comprises (a) the sequence of Hel308 Tga (i.e. SEQ ID NO: 33) or a variant thereof or (b) the sequence of Hel308 Csy (i.e. SEQ ID NO: 22) or a variant thereof or (c) the sequence of Hel308 Mhu (i.e. SEQ ID NO: 52) or a variant thereof. Variants of these sequences are discussed in more detail below. Variants preferably comprise one or more substituted cysteine residues and/or one or more substituted Faz residues to facilitate attachment as discussed above.

The helicase is preferably a Hel308 helicase. Any Hel308 helicase may be used in accordance with the invention. Hel308 helicases are also known as ski2-like helicases and the two terms can be used interchangeably. Suitable Hel308 helicases are disclosed in Table 4 of US Patent Application Nos. 61,549,998 and 61/599,244 and International Application No. PCT/GB2012/052579 (published as WO 2013/057495).

The Hel308 helicase typically comprises the amino acid motif Q-X1-X2-G-R-A-G-R (hereinafter called the Hel308 motif; SEQ ID NO: 8). The Hel308 motif is typically part of the helicase motif VI (Tuteja and Tuteja, Eur. J. Biochem. 271, 1849-1863 (2004)). X1 may be C, M or L. X1 is preferably C. X2 may be any amino acid residue. X2 is typically a hydrophobic or neutral residue. X2 may be A, F, M, C, V, L, I, S, T, P or R. X2 is preferably A, F, M, C, V, L, I, S, T or P. X2 is more preferably A, M or L. X2 is most preferably A or M.

The Hel308 helicase preferably comprises the motif Q-X1-X2-G-R-A-G-R-P (hereinafter called the extended Hel308 motif; SEQ ID NO: 9) wherein X1 and X2 are as described above.

The most preferred Hel308 helicases, Hel308 motifs and extended Hel308 motifs are shown in the Table 1 below.

TABLE 1
Preferred Hel308 helicases and their motifs

| | | | % | % | | |
|---|---|---|---|---|---|---|
| SEQ | | | Identity | Identity | | Extended |
| ID | | | Hel308 | Hel308 | Hel308 | Hel308 |
| NO: | Helicase | Names | Pfu | Mbu | motif | motif |
| 10 | Hel308 Mbu | Methanococcoides | 37% | —— | QMAGRAGR | QMAGRAGRP |
| | | burtonii | | | (SEQ ID NO: | (SEQ ID NO: 12) |
| | | | | | 11) | |
| 13 | Hel308 Pfu | Pyrococcus | — | 37% | QMLGRAGR | QMLGRAGRP |
| | | furiosus DSM | | | (SEQ ID NO: | (SEQ ID NO: 15) |
| | | 3638 | | | 14) | |
| 16 | Hel308 Hvo | Haloferax volcanii | 34% | 41% | QMMGRAGR | QMMGRAGRP |
| | | | | | (SEQ ID NO: | (SEQ ID NO: 18) |
| | | | | | 17) | |
| 19 | Hel308 Hla | Halorubrum | 35% | 42% | QMCGRAGR | QMCGRAGRP |
| | | lacusprofundi | | | (SEQ ID NO: | (SEQ ID NO: 21) |
| | | | | | 20) | |
| 22 | Hel308 Csy | Cenarchaeum | 34% | 34% | QLCGRAGR | QLCGRAGRP |
| | | symbiosum | | | (SEQ ID NO: | (SEQ ID NO: 24) |
| | | | | | 23) | |
| 25 | Hel308 Sso | Sulfolobus | 35% | 33% | QMSGRAGR | QMSGRAGRP |
| | | solfataricus | | | (SEQ ID NO: | (SEQ ID NO: 27) |
| | | | | | 26) | |
| 28 | Hel308 Mfr | Methanogenium | 37% | 44% | QMAGRAGR | QMAGRAGRP |
| | | frigidum | | | (SEQ ID NO: | (SEQ ID NO: 12) |
| | | | | | 11) | |
| 29 | Hel308 Mok | Methanothermococcus | 37% | 34% | QCIGRAGR | QCIGRAGRP |
| | | okinawensis | | | (SEQ ID NO: | (SEQ ID NO: 31) |
| | | | | | 30) | |
| 32 | Hel308 Mig | Methanotorris | 40% | 35% | QCIGRAGR | QCIGRAGRP |
| | | igneus Kol 5 | | | (SEQ ID NO: | (SEQ ID NO: 31) |
| | | | | | 30) | |
| 33 | Hel308 Tga | Thermococcus | 60% | 38% | QMMGRAGR | QMMGRAGRP |
| | | gammatolerans | | | (SEQ ID NO: | (SEQ ID NO: 18) |
| | | EJ3 | | | 17) | |
| 34 | Hel308 Tba | Thermococcus | 57% | 35% | QMIGRAGR | QMIGRAGRP |
| | | barophilus MP | | | (SEQ ID NO: | (SEQ ID NO: 36) |
| | | | | | 35) | |
| 37 | Hel308 Tsi | Thermococcus | 56% | 35% | QMMGRAGR | QMMGRAGRP |
| | | sibiricus MM 739 | | | (SEQ ID NO: | (SEQ ID NO: 18) |
| | | | | | 17) | |
| 38 | Hel308 Mba | Methanosarcina | 39% | 60% | QMAGRAGR | QMAGRAGRP |
| | | barkeri str. Fusaro | | | (SEQ ID NO: | (SEQ ID NO: 12) |
| | | | | | 11) | |
| 39 | Hel308 Mac | Methanosarcina | 38% | 60% | QMAGRAGR | QMAGRAGRP |
| | | acetivorans | | | (SEQ ID NO: | (SEQ ID NO: 12) |
| | | | | | 11) | |
| 40 | Hel308 | Methanohalophilus | 38% | 60% | QMAGRAGR | QMAGRAGRP |
| | Mmah | mahii DSM 5219 | | | (SEQ ID NO: | (SEQ ID NO: 12) |
| | | | | | 11) | |
| 41 | Hel308 | Methanosarcina | 38% | 60% | QMAGRAGR | QMAGRAGRP |
| | Mmaz | mazei | | | (SEQ ID NO: | (SEQ ID NO: 12) |
| | | | | | 11) | |
| 42 | Hel308 Mth | Methanosaeta | 39% | 46% | QMAGRAGR | QMAGRAGRP |
| | | thermophila PT | | | (SEQ ID NO: | (SEQ ID NO: 12) |
| | | | | | 11) | |
| 43 | Hel308 Mzh | Methanosalsum | 39% | 57% | QMAGRAGR | QMAGRAGRP |
| | | zhilinae DSM | | | (SEQ ID NO: | (SEQ ID NO: 12) |
| | | 4017 | | | 11) | |
| 44 | Hel308 Mev | Methanohalobium | 38% | 61% | QMAGRAGR | QMAGRAGRP |
| | | evestigatum Z- | | | (SEQ ID NO: | (SEQ ID NO: 12) |
| | | 7303 | | | 11) | |
| 45 | Hel308 | Methanococcus | 36% | 32% | QCIGRAGR | QCIGRAGRP |
| | Mma | maripaludis | | | (SEQ ID NO: | (SEQ ID NO: 31) |
| | | | | | 30) | |
| 46 | Hel308 Nma | Natrialba magadii | 37% | 43% | QMMGRAGR | QMMGRAGRP |
| | | | | | (SEQ ID NO: | (SEQ ID NO: 18) |
| | | | | | 17) | |
| 47 | Hel308 Mbo | Methanoregula | 38% | 45% | QMAGRAGR | QMAGRAGRP |
| | | boonei 6A8 | | | (SEQ ID NO: | (SEQ ID NO: 12) |
| | | | | | 11) | |
| 48 | Hel308 Fac | Ferroplasma | 34% | 32% | QMIGRAGR | QMIGRAGRP |
| | | acidarmanus | | | (SEQ ID NO: | (SEQ ID NO: 36) |
| | | | | | 35) | |
| 49 | Hel308 Mfe | Methanocaldococcus | 40% | 35% | QCIGRAGR | QCIGRAGRP |
| | | fervens AG86 | | | (SEQ ID NO: | (SEQ ID NO: 31) |
| | | | | | 30) | |
| 50 | Hel308 Mja | Methanocaldococcus | 24% | 22% | QCIGRAGR | QCIGRAGRP |
| | | jannaschii | | | (SEQ ID NO: | (SEQ ID NO: 31) |
| | | | | | 30) | |
| 51 | Hel308 Min | Methanocaldococcus | 41% | 33% | QCIGRAGR | QCIGRAGRP |
| | | infernus | | | (SEQ ID NO: | (SEQ ID NO: 31) |
| | | | | | 30) | |
| 52 | Hel308 Mhu | Methanospirillum | 36% | 40% | QMAGRAGR | QMAGRAGRP |
| | | hungatei JF-1 | | | (SEQ ID NO: | (SEQ ID NO: 12) |
| | | | | | 11) | |
| 53 | Hel308 Afu | Archaeoglobus | 40% | 40% | QMAGRAGR | QMAGRAGRP |
| | | fulgidus DSM | | | (SEQ ID NO: | (SEQ ID NO: 12) |
| | | 4304 | | | 11) | |
| 54 | Hel308 Htu | Haloterrigena | 35% | 43% | QMAGRAGR | QMMGRAGRP |
| | | turkmenica | | | (SEQ ID NO: | (SEQ ID NO: 12) |
| | | | | | 11) | |
| 55 | Hel308 Hpa | Haladaptatus | 38% | 45% | QMFGRAGR | QMFGRAGRP |
| | | paucihalophilus | | | (SEQ ID NO: | (SEQ ID NO: 57) |
| | | DX253 | | | 56) | |
| 58 | Hel308 Hsp | Halobacterium sp. | 36.8% | 42.0% | QMFGRAGR | QMFGRAGRP |
| | ski2-like | NRC-1 | | | (SEQ ID | (SEQ ID NO: 57) |
| | helicase | | | | NO:56) | |

The most preferred Hel308 motif is shown in SEQ ID NO: 17. The most preferred extended Hel308 motif is shown in SEQ ID NO: 18.

The Hel308 helicase preferably comprises the sequence of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 or a variant thereof.

In Hel308 helicases, the polynucleotide domain and opening can be found between domain 2 (one of the ATPase domains) and domain 4 (the ratchet domain) and domain 2 and domain 5 (the molecular brake). The two or more parts connected in accordance with the invention are preferably (a) any amino acid in domain 2 and any amino acid in domain 4 or (b) any amino acid in domain 2 and any amino acid in domain 5. The amino acid residues which define domains 2, 4 and 5 in various Hel308 helicases are listed in Table 2 below.

TABLE 2
Amino acid residues which correspond to
domains 2, 4 and 5 in various Hel 308 helicases

| SEQ | Hel308 | Domain 2 | | Domain 4 | | Domain 5 | |
|---|---|---|---|---|---|---|---|
| ID NO: | Homologue | Start | End | Start | End | Start | End |
| 10 | Mbu | W200 | E409 | Y506 | G669 | S670 | Q760 |
| 13 | Pfu | W198 | F398 | Y490 | G640 | I641 | S720 |
| 16 | Hvo | W201 | W418 | Y509 | G725 | V726 | E827 |
| 19 | Hla | W201 | W418 | Y513 | G725 | V726 | R824 |
| 22 | Csy | W205 | G414 | Y504 | G644 | I645 | K705 |
| 25 | Sso | W204 | L420 | Y506 | G651 | I652 | S717 |
| 28 | Mfr | W193 | E397 | Y488 | G630 | I631 | I684 |
| 29 | Mok | W198 | G415 | Y551 | G706 | A707 | I775 |
| 32 | Mig | W200 | E408 | Y495 | G632 | A633 | I699 |
| 33 | Tga | W198 | R399 | Y491 | G639 | V640 | R720 |
| 34 | Tba | W219 | F420 | Y512 | G660 | V661 | K755 |
| 37 | Tsi | W221 | L422 | Y514 | G662 | V663 | K744 |
| 38 | Mba | W200 | E409 | Y498 | G643 | A644 | Y729 |
| 39 | Mac | W200 | E409 | Y499 | G644 | A645 | F730 |
| 40 | Mmah | W196 | G405 | Y531 | G678 | A679 | N747 |
| 41 | Mmaz | W200 | E409 | Y499 | G644 | A645 | Y730 |
| 42 | Mth | W203 | M404 | Y491 | G629 | A630 | A693 |
| 43 | Mzh | W200 | N409 | Y505 | G651 | I652 | T739 |
| 44 | Mev | W200 | D409 | Y499 | G643 | V644 | F733 |
| 45 | Mma | W196 | G405 | Y531 | G678 | A679 | N747 |
| 46 | Nma | W201 | W413 | Y541 | G688 | V689 | F799 |
| 47 | Mbo | W197 | E402 | Y493 | G637 | I638 | G723 |
| 48 | Fac | F197 | T390 | Y480 | G613 | V614 | R681 |
| 49 | Mfe | W199 | Q408 | Y494 | G629 | A630 | F696 |
| 50 | Mja | W197 | Q406 | Y492 | G627 | A628 | F694 |
| 51 | Min | W189 | Q390 | Y476 | G604 | A605 | I670 |
| 52 | Mhu | W198 | D402 | Y493 | G637 | V638 | C799 |
| 53 | Afu | W201 | F399 | Y487 | G626 | V627 | E696 |
| 54 | Htu | W201 | W413 | Y533 | G680 | V681 | F791 |
| 55 | Hpa | W201 | W412 | Y502 | G657 | V658 | E752 |
| 58 | Hsp | W210 | Y421 | Y512 | G687 | V688 | S783 |
| | (ski2-like | | | | | | |
| | helicase) | | | | | | |

The Hel308 helicase preferably comprises the sequence of Hel308 Mbu (i.e. SEQ ID NO: 10) or a variant thereof. In Hel308 Mbu, the polynucleotide domain and opening can be found between domain 2 (one of the ATPase domains) and domain 4 (the ratchet domain) and domain 2 and domain 5 (the molecular brake). The two or more parts of Hel308 Mbu connected are preferably (a) any amino acid in domain 2 and any amino acid in domain 4 or (b) any amino acid in domain 2 and any amino acid in domain 5. The amino acid residues which define domains 2, 4 and 5 for Hel308 Mbu are listed in Table 2 above. The two or more parts of Hel308 Mbu connected are preferably amino acids 284 and 615 in SEQ ID NO: 10. These amino acids are preferably substituted with cysteine (i.e. E284C and S615C) such that they can be connected by cysteine linkage.

The invention also provides a mutant Hel308 Mbu protein which comprises a variant of SEQ ID NO: 10 in which E284 and S615 are modified. E284 and S615 are preferably substituted. E284 and S615 are more preferably substituted with cysteine (i.e. E284C and S615C). The variant may differ from SEQ ID NO: 10 at positions other than E284 and S615 as long as E284 and S615 are modified. The variant will preferably be at least 30% homologous to SEQ ID NO: 10 based on amino acid identity as discussed in more detail below. E284 and S615 are not connected. The mutant Hel308 Mbu protein of the invention may be used to form a modified helicase of the invention in which E284 and S615 are connected.

The Hel308 helicase more preferably comprises (a) the sequence of Hel308 Tga (i.e. SEQ ID NO: 33) or a variant thereof, (b) the sequence of Hel308 Csy (i.e. SEQ ID NO: 22) or a variant thereof or (c) the sequence of Hel308 Mhu (i.e. SEQ ID NO: 52) or a variant thereof.

A variant of a Hel308 helicase is an enzyme that has an amino acid sequence which varies from that of the wild-type helicase and which retains polynucleotide binding activity. In particular, a variant of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 is an enzyme that has an amino acid sequence which varies from that of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58and which retains polynucleotide binding activity. Polynucleotide binding activity can be determined using methods known in the art. Suitable methods include, but are not limited to, fluorescence anisotropy, tryptophan fluorescence and electrophoretic mobility shift assay (EMSA). For instance, the ability of a variant to bind a single stranded polynucleotide can be determined as described in the Examples.

The variant retains helicase activity. This can be measured in various ways. For instance, the ability of the variant to translocate along a polynucleotide can be measured using electrophysiology, a fluorescence assay or ATP hydrolysis.

The variant may include modifications that facilitate handling of the polynucleotide encoding the helicase and/or facilitate its activity at high salt concentrations and/or room temperature. Variants typically differ from the wild-type helicase in regions outside of the Hel308 motif or extended Hel308 motif discussed above. However, variants may include modifications within these motif(s).

Over the entire length of the amino acid sequence of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58, a variant will preferably be at least 30% homologous to that sequence based on amino acid identity. More preferably, the variant polypeptide may be at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described below. The variant may differ from the wild-type sequence in any of the ways discussed below with reference to SEQ ID NOs: 2 and 4.

A variant of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 preferably comprises the Hel308 motif or extended Hel308 motif of the wild-type sequence as shown in Table 1 above. However, a variant may comprise the Hel308 motif or extended Hel308 motif from a different wild-type sequence. For instance, a variant of SEQ ID NO: 10 may comprise the Hel308 motif or extended Hel308 motif from SEQ ID NO: 13 (i.e. SEQ ID NO: 14 or 15). Variants of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 may also include modifications within the Hel308 motif or extended Hel308 motif of the relevant wild-type sequence. Suitable modifications at X1 and X2 are discussed above when defining the two motifs. A variant of SEQ ID NO: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 or 58 preferably comprises one or more substituted cysteine residues and/or one or more substituted Faz residues to facilitate attachment as discussed above.

A variant of SEQ ID NO: 10 may lack the first 19 amino acids of SEQ ID NO: 10 and/or lack the last 33 amino acids of SEQ ID NO: 10. A variant of SEQ ID NO: 10 preferably comprises a sequence which is at least 70%, at least 75%, at least 80%, at least 85%, at least 90% or more preferably at least 95%, at least 97% or at least 99% homologous based on amino acid identity with amino acids 20 to 211 or 20 to 727 of SEQ ID NO: 10.

SEQ ID NO: 10 (Hel308 Mbu) contains five natural cysteine residues. However, all of these residues are located within or around the DNA binding grove of the enzyme. Once a DNA strand is bound within the enzyme, these natural cysteine residues become less accessible for external modifications. This allows specific cysteine mutants of SEQ ID NO: 10 to be designed and attached to the moiety using cysteine linkage as discussed above. Preferred variants of SEQ ID NO: 10 have one or more of the following substitutions: A29C, Q221C, Q442C, T569C, A577C, A700C and S708C. The introduction of a cysteine residue at one or more of these positions facilitates cysteine linkage as discussed above. Other preferred variants of SEQ ID NO: 10 have one or more of the following substitutions: M2Faz, R10Faz, F15Faz, A29Faz, R185Faz, A268Faz, E284Faz, Y387Faz, F400Faz, Y455Faz, E464Faz, E573Faz, A577Faz, E649Faz, A700Faz, Y720Faz, Q442Faz and S708Faz. The introduction of a Faz residue at one or more of these positions facilitates Faz linkage as discussed above.

The helicase is preferably a RecD helicase. Any RecD helicase may be used in accordance with the invention. The structures of RecD helicases are known in the art (FEBS J. 2008 Apr.; 275(8):1835-51L Epub 2008 Mar. 9, ATPase activity of RecD is essential for growth of the Antarctic Pseudomonas syringae Lz4W at low temperature. Satapathy A K. Pavankumar T L, Bhattacharjya S, Sankaranarayanan R, Ray M K; EMS Microbiol Rev. 2009 May; 33(3):657-87. The diversity of conjugative relaxases and its application in plasmid classification. Garcillán-Barcia M P, Francia M V, de la Cruz F; J Biol Chem. 2011 Apr. 8; 286(14):12670-82. Epub 2011 Feb. 2. Functional characterization of the multidomain F plasmid TraI relaxase-helicase. Cheng Y, McNamara D E, Miley M J, Nash R P, Redinbo M R).

The RecD helicase typically comprises the amino acid motif X1-X2-X3-G-X4-X5-X6-X7 (hereinafter called the RecD-like motif I; SEQ ID NO: 59), wherein X1 is G, S or A, X2 is any amino acid, X3 is P, A, S or G, X4 is T, A, V, S or C, X5 is G or A, X6 is K or R and X7 is T or S. X1 is preferably G. X2 is preferably G, I, Y or A. X2 is more preferably G. X3 is preferably P or A. X4 is preferably T, A, V or C. X4 is preferably T, V or C. X5 is preferably G. X6 is preferably K. X7 is preferably T or S. The RecD helicase preferably comprises Q-(X8)₁₆₋₁₈-X1-X2-X3-G-X4-X5-X6-X7 (hereinafter called the extended RecD-like motif I; SEQ ID NOs 60, 61 and 62), wherein X1 to X7 are as defined above and X8 is any amino acid. There are preferably 16 X8 residues (i.e. (X8)₁₆) in the extended RecD-like motif I (SEQ ID NO: 60). Suitable sequences for (X8)¹⁶ can be identified in SEQ ID NOs: 14, 17, 20, 23, 26, 29, 32, 35, 38, 41, 44, 47 and 50 of U.S. Patent Application No. 61/581,332 and SEQ ID NOs: 18, 21, 24, 25, 28, 30, 35, 37, 39, 41, 42 and 44 of International Application No. PCT/GB2012/053274 (published as WO 2012/098562).

The RecD helicase preferably comprises the amino acid motif G-G-P-G-Xa-G-K-Xb (hereinafter called the RecD motif I; SEQ ID NO: 63) wherein Xa is T, V or C and Xb is T or S. Xa is preferably T. Xb is preferably T. The Rec-D helicase preferably comprises the sequence G-G-P-G-T-G-K-T (SEQ ID NO: 64). The RecD helicase more preferably comprises the amino acid motif Q-(X8)₁₆₋₁₈-G-G-P-G-Xa-G-K-Xb (hereinafter called the extended Reel) motif 1: SEQ ID NO: 65, 66 and 67), wherein Xa and Xb are as defined above and X8 is any amino acid. There are preferably 16 X8 residues (i.e. (X8)₁₆) in the extended RecD motif I (SEQ I) NO: 65). Suitable sequences for (X8)₁₆ can be identified in SEQ ID NOs: 14, 17, 20, 23, 26, 29, 32, 35, 38, 41, 44, 47 and 50 of U.S. Patent Application No. 61/581,332 and SEQ ID NOs: 18, 21, 24, 25, 28, 30, 32, 35, 37, 39, 41, 42) and 44 of International Application No. PCT/GB2012/053274 (published as WO 2012/098562).

The RecD helicase typically comprises the amino acid motif X1-X2-X3-X4-X5-(X6)₃-Q-X7 (hereinafter called the RecD-like motif V; SEQ ID NO: 68), wherein XI is Y, W or F, X2 is A, T, S, M, C or V, X3 is any amino acid, X4 is T, N or S. X5 is A, T, G, S, V or 1, X6 is any amino acid and X7 is G or S, XI is preferably Y. X2 is preferably A, M, C or V. X2 is more preferably A. X3 is preferably 1, M or L. X3 is more preferably I or L. X4 is preferably T or S. X4 is more preferably T. X5 is preferably A, V or I. X5 is more preferably V or I. X5 is most preferably V. (X6)₃ is preferably H-K-S, H-M-A, H-G-A or H-R-S. (X6)₃ is more preferably H-K-S. X7 is preferably G. The RecD helicase preferably comprises the amino acid motif Xa-Xb-Xc-Xd-Xe-H-K-S-Q-G (hereinafter called the RecD motif V; SEQ ID NO: 69), wherein Xa is Y, W or F, Xb is A, M, C or V, Xc is I, M or L, Xd is T or S and Xe is V or I. Xa is preferably Y. Xb is preferably A. Xd is preferably T. Xd is preferably V. Preferred RecD motifs I are shown in Table 5 of U.S. Patent Application No. 61/581,332, Preferred RecD-like motifs I are shown in Table 7 of U.S. Patent Application No. 61/581,332 and International Application No. PCT/GB2012/053274 (published as WO 2012/098562). Preferred RecD-like motifs V are shown in Tables 5 and 7 of U.S. Patent Application No. 61/581,332 and International Application No. PCT/GB2012/053274 (published as WO 2012/098562).

The RecD helicase is preferably one of the helicases shown in Table 4 or 5 of U.S. Patent Application No. 61/581,332 and International Application No. PCT/GB2012/053274 (published as WO 2012/098562) or a variant thereof. Variants are described in U.S. Patent Application No. 61/581,332 and International Application No. PCT/GB2012/053274 (published as WO 2012/098562).

The RecD helicase is preferably a TraI helicase or a TraI subgroup helicase. TraI helicases and TraI subgroup helicases may contain two RecD helicase domains, a relaxase domain and a C-terminal domain. The TraI subgroup helicase is preferably a TrwC helicase. The TraI helicase or TraI subgroup helicase is preferably one of the helicases shown in Table 6 of U.S. Patent Application No. 61/581,332 and International Application No. PCT/GB2012/053274 (published as WO 2012/098562) or a variant thereof. Variants are described in U.S. Patent Application No. 61/581,332 and International Application No. PCT/GB2012/053274 (published as WO 2012/098562).

The TraI helicase or a TraI subgroup helicase typically comprises a RecD-like motif I as defined above (SEQ ID NO: 59) and/or a RecD-like motif V as defined above (SEQ ID NO: 68). The TraI helicase or a TraI subgroup helicase preferably comprises both a RecD-like motif I (SEQ ID NO: 59) and a RecD-like motif V (SEQ ID NO: 68). The TraI helicase or a TraI subgroup helicase typically further comprises one of the following two motifs:
The amino acid motif H-(X1)₂-X2-R-(X3)₅₋₁₂-H-X4-H (hereinafter called the MobF motif III; SEQ ID NOs: 70 to 77), wherein X1 and X2 are any amino acid and X2 and X4 are independently selected from any amino acid except D, E, K and R, (X1)₂ is of course X1a-X1b. X1a and X1b can be the same of different amino acid. X1a is preferably D or E. X1b is preferably T or D. (X1)₂ is preferably DT or ED. (X1)₂ is most preferably DT. The 5 to 12 amino acids in (X3)₅₋₁₂ can be the same or different. X2 and X4 are independently selected from G, P, A, V, L, I, M, C, F, Y, W, H, Q, N, S and T. X2 and X4 are preferably not charged. X2 and X4 are preferably not H. X2 is more preferably N. S or A. X2 is most preferably N. X4 is most preferably F or T. (X3)₅₋₁₂₂ is preferably 6 or 10 residues in length. Suitable embodiments of (X3)₅₋₁₂ can be derived from SEQ ID NOs: 58, 62, 66 and 70 shown in Table 7 of U.S. Patent Application No. 61/581,332 and SEQ ID NOs: 61, 65, 69, 73, 74, 82, 86, 90, 94, 98, 102, 110, 112, 113, 114, 117, 121, 124, 125, 129, 133, 136, 140, 144, 147, 151, 152, 156, 160, 164 and 168 of International Application No. PCT/GB2012/053274 (published as WO 2012/098562).
The amino acid motif G-X1-X2-X3-X4-X5-X6-X7-H-(X8)₆₋₁₂-H-X9 (hereinafter called the MobQ motif III; SEQ ID NOs: 78 to 84), wherein X1, X2, X3, X5, X6, X7 and X9 are independently selected from any amino acid except D, E, K and R, X4 is D or E and X8 is any amino acid. X1, X2, X3, X5, X6, X7 and X9 are independently selected from G, P, A, V, L, I, M, C, F, Y, W, H, Q, N, S and T. X1, X2, X3, X5, X6, X7 and X9 are preferably not charged. X1, X2, X3, X5, X6, X7 and X9 are preferably not H. The 6 to 12 amino acids in (X8)₆₋₁₂ can be the same or different. Preferred MobF motifs III are shown in Table 7 of U.S. Patent Application No. 61/581,332 and International Application No. PCT/GB2012/053274 (published as WO 2012/098562).

The TraI helicase or TraI subgroup helicase is more preferably one of the helicases shown in Table 6 or 7 of U.S. Patent Application No. 61/581,332 and International Application No. PCT/GB2012/053274 (published as WO 2012/098562) or a variant thereof. The TraI helicase most preferably comprises the sequence shown in SEQ ID NO: 85 or a variant thereof. SEQ ID NO: 85 is TraI Eco (NCBI Reference Sequence: NP_061483.1; Genbank AAQ98619.1; SEQ ID NO: 85). TraI Eco comprises the following motifs: RecD-like motif I (GYAGVGKT; SEQ ID NO: 86), RecD-like motif V (YAITAHGAQG; SEQ ID NO: 87) and Mob F motif III (HDTSRDQEPQLHTH; SEQ ID NO: 88).

The TraI helicase or TraI subgroup helicase more preferably comprises the sequence of one of the helicases shown in Table 3 below, i.e. one of SEQ ID NOs: 85, 126, 134 and 138, or a variant thereof.

TABLE 3
More preferred TraI helicase and TraI subgroup helicases

| | | | | | RecD- | RecD- | Mob F |
|---|---|---|---|---|---|---|---|
| | | | | % | like | like | motif |
| SEQ | | | | Identity | motif I | motif V | III |
| ID | | | | to TraI | (SEQ ID | (SEQ ID | (SEQ ID |
| NO | Name | Strain | NCBI ref | Eco | NO:) | NO:) | NO:) |
| 85 | TraI | Escherichia | NCBI | - | GYAGV | YAITA | HDTSR |
| | Eco | coli | Reference | | GKT | HGAQG | DQEPQ |
| | | | Sequence: | | (86) | (87) | LHTH |
| | | | NP_061483.1 | | | | 88) |
| | | | Genbank | | | | |
| | | | AAQ98619.1 | | | | |
| 126 | TrwC | Citromicrobium | NCBI | 15% | GIAGA | YALNV | HDTNR |
| | Cba | bathyomarinum | Reference | | GKS | HMAQG | NQEPN |
| | | JL354 | Sequence: | | (131) | (132) | LHFH |
| | | | ZP_06861556.1 | | | | (133) |
| 134 | TrwC | Halothiobacillus | NCBI | 11.5% | GAAGA | YCITIH | HEDAR |
| | Hne | neapolitanus | Reference | | GKT | RSQG | TVDDI |
| | | c2 | Sequence: | | (135) | (136) | ADPQL |
| | | | YP_003262832.1 | | | | HTH |
| | | | | | | | (137) |
| 138 | TrwC | Erythrobacter | NCBI | 16% | GIAGA | YALNA | HDTNR |
| | Eli | litoralis | Reference | | GKS | HMAQG | NQEPN |
| | | HTCC2594 | Sequence: | | (131) | (139) | LHFH |
| | | | YP_457045.1 | | | | (133) |

The two or more parts of TrwC Cba connected are preferably (a) amino acids 691 and 346 in SEQ ID NO: 126; (b) amino acids 657 and 339 in SEQ ID NO: 126; (c) amino acids 691 and 350 in SEQ ID NO: 126; or (d) amino acids 690 and 350 in SEQ ID NO: 126. These amino acids are preferably substituted with cysteine such that they can be connected by cysteine linkage.

The invention also provides a mutant TrwC Cba protein which comprises a variant of SEQ ID NO: 126 in which amino acids 691 and 346; 657 and 339; 691 and 350; or 690 and 350 are modified. The amino acids are preferably substituted. The amino acids are more preferably substituted with cysteine. The variant may differ from SEQ ID NO: 126 at positions other than 691 and 346; 657 and 339; 691 and 350; or 690 and 350 as long as the relevant amino acids are modified. The variant will preferably be at least 10% homologous to SEQ ID NO: 126 based on amino acid identity as discussed in more detail below. Amino acid 691 and 346; 657 and 339; 691 and 350; or 690 and 350 are not connected. The mutant TrwC Cba protein of the invention may be used to form a modified helicase of the invention in which the modified amino acids are connected.

A variant of a RecD helicase, TraI helicase or TraI subgroup helicase is an enzyme that has an amino acid sequence which varies from that of the wild-type helicase and which retains polynucleotide binding activity. This can be measured as described above. In particular, a variant of SEQ ID NO: 85, 126, 134 or 138 is an enzyme that has an amino acid sequence which varies from that of SEQ ID NO: 85, 126, 134 or 138 and which retains polynucleotide binding activity. The variant retains helicase activity. The variant must work in at least one of the two modes discussed below. Preferably, the variant works in both modes. The variant may include modifications that facilitate handling of the polynucleotide encoding the helicase and/or facilitate its activity at high salt concentrations and/or room temperature. Variants typically differ from the wild-type helicase in regions outside of the motifs discussed above. However, variants may include modifications within these motif(s).

Over the entire length of the amino acid sequence of any one of SEQ ID NO: 85, 126, 134 and 138, a variant will preferably be at least 10% homologous to that sequence based on amino acid identity. More preferably, the variant polypeptide may be at least 20%, at least 25%, at least 30%, at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 85, 126, 134 and 138 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described below. The variant may differ from the wild-type sequence in any of the ways discussed above with reference to SEQ ID NOs: 2 and 4.

A variant of any one of SEQ ID NOs: 85, 126, 134 and 138 preferably comprises the RecD-like motif I and/or RecD-like motif V of the wild-type sequence. However, a variant of SEQ ID NO: 85, 126, 134 or 138 may comprise the RecD-like motif I and/or extended RecD-like motif V from a different wild-type sequence. For instance, a variant may comprise any one of the preferred motifs shown in Tables 5 and 7 of U.S. Patent Application No. 61/581,332 and International Application No. PCT/GB2012/053274 (published as WO 2012/098562). Variants of SEQ ID NOs: 85, 126, 134 and 138 may also include modifications within the RecD-like motifs I and V of the wild-type sequence. A variant of SEQ ID NO: 85, 126, 134 or 138 preferably comprises one or more substituted cysteine residues and/or one or more substituted Faz residues to facilitate attachment as discussed above.

The helicase is preferably an XPD helicase. Any XPD helicase may be used in accordance with the invention. XPD helicases are also known as Rad3 helicases and the two terms can be used interchangeably.

The structures of XPD helicases are known in the art (Cell. 2008 May 30; 133(5):801-12. Structure of the DNA repair helicase XPD, Liu H, Rudolf J, Johnson A, McMahon S A, Oke M, Carter L, McRobbie A M, Brown S E, Naismith J H, White M F). The XPD helicase typically comprises the amino acid motif X1-X2-X3-G-X4-X5-X6-E-G (hereinafter called XPD motif V; SEQ ID NO: 89). X1, X2, X5 and X6 are independently selected from any amino acid except D, E, K and R. X1, X2, X5 and X6 are independently selected from G, P, A, V, L, I, M, C, F, Y, W, H, Q, N, S and T. X1, X2, X5 and X6 are preferably not charged. X1, X2, X5 and X6 are preferably not H. X1 is more preferably V, L, I, S or Y. X5 is more preferably V, L, I, N or F. X6 is more preferably S or A. X3 and X4 may be any amino acid residue. X4 is preferably K, R or T.

The XPD helicase typically comprises the amino acid motif Q-Xa-Xb-G-Xc-Xd-(Xe)₃-Xf-(Xg)₇-D-Xh-R (hereinafter called XPD motif VI; SEQ ID NO: 90). Xa, Xe and Xg may be any amino acid residue. Xb, Xc and Xd are independently selected from any amino acid except D, E, K and R. Xb, Xc and Xd are typically independently selected from G, P, A, V, L, I, M, C, F, Y, W, H, Q, N, S and T. Xb, Xc and Xd are preferably not charged. Xb, Xc and Xd are preferably not H. Xb is more preferably V, A, L, I or M. Xc is more preferably V, A, L, I, M or C. Xd is more preferably I, H, L, F, M or V. Xf may be D or E. (Xg)₇ is X_{g1}, X_{g2}, X_{g3}, X_{g4}, X_{g5}, X_{g6} and X_{g7}. X_{g2} is preferably G, A, S or C. X_{g5} is preferably F, V, L I, M, A, W or Y. X_{g6} is preferably L, F, Y, M, I or V, X_{g7} is preferably A, C, V, L, I, M or S.

The XPD helicase preferably comprises XPD motifs V and VI. The most preferred XPD motifs V and VI are shown in Table 5 of U.S. Patent Application No. 61/581,340 and International Application No. PCT/GB2012/053273 (published as WO 2012/098561).

The XPD helicase preferably further comprises an iron sulphide (FeS) core between two Walker A and B motifs (motifs I and II). An FeS core typically comprises an iron atom coordinated between the sulphide groups of cysteine residues. The FeS core is typically tetrahedral.

The XPD helicase is preferably one of the helicases shown in Table 4 or 5 of U.S. Patent Application No. 61/581,340 and International Application No. PCT/GB2012/053273 (published as WO 2012/098561) or a variant thereof. The XPD helicase most preferably comprises the sequence shown in SEQ ID NO: 91 or a variant thereof. SEQ ID NO: 91 is XPD Mbu (Methanococcoides burtonii; YP_566221.1; GI:91773529). XPD Mbu comprises YLWGTLSEG (Motif V; SEQ ID NO: 92) and QAMGRVVRSPTDYGARILLDGR (Motif VI; SEQ ID NO: 93).

A variant of a XPD helicase is an enzyme that has an amino acid sequence which varies from that of the wild-type helicase and which retains polynucleotide binding activity. This can be measured as described above. In particular, a variant of SEQ ID NO: 91 is an enzyme that has an amino acid sequence which varies from that of SEQ ID NO: 91 and which retains polynucleotide binding activity. The variant retains helicase activity. The variant must work in at least one of the two modes discussed below. Preferably, the variant works in both modes. The variant may include modifications that facilitate handling of the polynucleotide encoding the helicase and/or facilitate its activity at high salt concentrations and/or room temperature. Variants typically differ from the wild-type helicase in regions outside of XPD motifs V and VI discussed above. However, variants may include modifications within one or both of these motifs.

Over the entire length of the amino acid sequence of SEQ ID NO: 91, such as SEQ ID NO: 10, a variant will preferably be at least 10%, preferably 30% homologous to that sequence based on amino acid identity. More preferably, the variant polypeptide may be at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 91 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described below. The variant may differ from the wild-type sequence in any of the ways discussed above with reference to SEQ ID NOs: 2 and 4.

A variant of SEQ ID NO: 91 preferably comprises the XPD motif V and/or the XPD motif VI of the wild-type sequence. A variant of SEQ ID NO: 91 more preferably comprises both XPD motifs V and VI of SEQ ID NO: 91. However, a variant of SEQ ID NO: 91 may comprise XPD motifs V and/or VI from a different wild-type sequence. For instance, a variant of SEQ ID NO: 91 may comprise any one of the preferred motifs shown in Table 5 of U.S. Patent Application No. 61/581,340 and International Application No. PCT/GB2012/053273 (published as WO 2012/098561). Variants of SEQ ID NO: 91 may also include modifications within XPD motif V and/or XPD motif VI of the wild-type sequence. Suitable modifications to these motifs are discussed above when defining the two motifs. A variant of SEQ ID NO: 91 preferably comprises one or more substituted cysteine residues and/or one or more substituted Faz residues to facilitate attachment as discussed above.

### Modified Hel308 Helicases

The present invention also provides a modified Hel308 helicase that is useful for controlling the movement of a polynucleotide. In accordance with the invention, the helicase is modified by the introduction of one or more cysteine residues and/or one or more non-natural amino acids at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10), wherein the helicase retains its ability to control the movement of a polynucleotide. The one or more cysteine residues and/or one or more non-natural amino acids are preferably introduced by substitution.

These modifications do not prevent the helicase from binding to a polynucleotide. For instance, the helicase may bind to a polynucleotide via internal nucleotides or at one of its termini. These modifications decrease the ability of the polynucleotide to unbind or disengage from the helicase, particularly from internal nucleotides of the polynucleotide. In other words, the one or more modifications increase the processivity of the Hel308 helicase by preventing dissociation from the polynucleotide strand. The thermal stability of the enzyme is also increased by the one or more modifications giving it an improved structural stability that is beneficial in Strand Sequencing. The modified Hel308 helicases of the invention have all of the advantages and uses discussed above.

The modified Hel308 helicase has the ability to control the movement of a polynucleotide. This can be measured as discussed above. The modified Hel308 helicase is artificial or non-natural.

A modified Hel308 helicase of the invention may be isolated, substantially isolated, purified or substantially purified as discussed above.

The Hel308 helicase preferably comprises a variant of one of the helicases shown in Table 1 above which comprises one or more cysteine residues and/or one or more non-natural amino acids at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10).

The Hel308 helicase preferably comprises a variant of one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 which comprises one or more cysteine residues and/or one or more non-natural amino acids at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10).

The Hel308 helicase preferably comprises a variant of one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 which comprises one or more cysteine residues and/or one or more non-natural amino acids at one or more of the positions which correspond to D274, E284, E285, E287, S288, T289, G290, E291, N316, K319, S615, K717 or Y720 in Hel308 Mbu (SEQ ID NO: 10).

TABLE 4a
Positions which correspond to D274, E284, E285, S288, S615,
K717 and Y720 in Hel308 Mbu (SEQ ID NO: 10).

| SEQ | Hel308 | | | | | | | |
|---|---|---|---|---|---|---|---|---|
| ID NO: | homologue | A | B | C | D | E | F | G |
| 10 | Mbu | D274 | E284 | E285 | S288 | S615 | K717 | Y720 |
| 13 | Pfu | L265 | E275 | L276 | S279 | P585 | K690 | E693 |
| 16 | Hvo | E283 | E293 | I294 | V297 | D671 | K775 | E778 |
| 19 | Hla | E283 | E293 | I294 | G297 | D668 | R775 | E778 |
| 22 | Csy | D280 | K290 | I291 | S294 | P589 | T694 | N697 |
| 25 | Sso | L281 | K291 | Q292 | D295 | D596 | K702 | Q705 |
| 28 | Mfr | H264 | E272 | K273 | A276 | G576 | K678 | E681 |
| 29 | Mok | S279 | L289 | S290 | D293 | P649 | K753 | R756 |
| 32 | Mig | Y276 | L286 | S287 | D290 | P579 | K679 | K682 |
| 33 | Tga | L266 | S276 | L277 | Q280 | P583 | K689 | D692 |
| 34 | Tba | L287 | E297 | L298 | S301 | S604 | K710 | E713 |
| 37 | Tsi | L289 | Q299 | L300 | G303 | N606 | G712 | E715 |
| 38 | Mba | E274 | D284 | E285 | E288 | S589 | K691 | D694 |
| 39 | Mac | E274 | D284 | E285 | E288 | P590 | K692 | E695 |
| 40 | Mmah | H272 | L282 | S283 | D286 | P621 | K725 | K728 |
| 41 | Mmaz | E274 | D284 | E285 | E288 | P590 | K692 | E698 |
| 42 | Mth | A269 | L279 | A280 | L283 | H575 | K677 | E680 |
| 43 | Mzh | H274 | Q284 | E285 | E288 | P596 | K699 | Q702 |
| 44 | Mev | G274 | E284 | E285 | E288 | T590 | K691 | Y694 |
| 45 | Mma | H272 | L282 | S283 | D286 | P621 | K725 | K728 |
| 46 | Nma | G277 | T287 | E288 | E291 | D634 | K737 | E740 |
| 47 | Mbo | A270 | E277 | R278 | E281 | S583 | G685 | E688 |
| 48 | Fac | Q264 | F267 | E268 | E271 | P559 | K663 | K666 |
| 49 | Mfe | R275 | L285 | S286 | E289 | P576 | K676 | K679 |
| 50 | Mja | I273 | L283 | S284 | E287 | P574 | K674 | K677 |
| 51 | Min | R257 | L267 | S268 | D271 | P554 | K651 | K654 |
| 52 | Mhu | S269 | Q277 | E278 | R281 | S583 | G685 | R688 |
| 53 | Afu | K268 | K277 | A278 | E281 | D575 | R677 | E680 |
| 54 | Htu | D277 | D287 | D288 | D291 | D626 | K729 | E732 |
| 55 | Hpa | D276 | D286 | Q287 | D290 | D595 | K707 | E710 |
| 58 | Hsp | E286 | E296 | I297 | V300 | D633 | A737 | E740 |
| | (ski2-like | | | | | | | |
| | helicase) | | | | | | | |

TABLE 4b
Positions which correspond to E287, T289, G290,
E291, N316 and K319 in Hel308 Mbu (SEQ ID NO: 10).

| SEQ | Hel308 | | | | | | |
|---|---|---|---|---|---|---|---|
| ID NO: | homologue | H | I | J | K | L | M |
| 10 | Mbu | E287 | T289 | G290 | E291 | N316 | K319 |
| 13 | Pfu | D278 | L280 | E281 | E282 | D307 | V310 |
| 16 | Hvo | D296 | S298 | D299 | T300 | E324 | T327 |
| 19 | Hla | S296 | S298 | D299 | T300 | E324 | A327 |
| 22 | Csy | S293 | G295 | G296 | E297 | D322 | S325 |
| 25 | Sso | D294 | I296 | E297 | E298 | A325 | D328 |
| 28 | Mfr | E275 | A277 | A278 | E279 | M304 | T307 |
| 29 | Mok | L292 | N294 | P295 | T296 | E320 | K323 |
| 32 | Mig | L289 | P291 | P292 | T293 | E317 | K320 |
| 33 | Tga | S279 | L281 | E282 | D283 | V308 | T311 |
| 34 | Tba | E300 | L302 | E303 | S304 | A329 | T332 |
| 37 | Tsi | D302 | L304 | D305 | T306 | T331 | S334 |
| 38 | Mba | L287 | N289 | S290 | E291 | P316 | E319 |
| 39 | Mac | L287 | N289 | S290 | E291 | P316 | E319 |
| 40 | Mmah | L285 | R287 | P288 | V289 | K313 | K316 |
| 41 | Mmaz | I287 | N289 | S290 | E291 | P316 | E319 |
| 42 | Mth | R282 | S284 | G285 | E286 | E311 | R314 |
| 43 | Mzh | G287 | A289 | G290 | E291 | E316 | R319 |
| 44 | Mev | L287 | T289 | S290 | D291 | A316 | K319 |
| 45 | Mma | L285 | R287 | P288 | V289 | K313 | K316 |
| 46 | Nma | R290 | D292 | S293 | D294 | T319 | S322 |
| 47 | Mbo | L280 | G282 | T283 | P284 | K309 | S312 |
| 48 | Fac | L270 | I272 | P273 | P274 | D299 | T302 |
| 49 | Mfe | L288 | P290 | P291 | T292 | Q316 | K319 |
| 50 | Mja | L286 | P288 | P289 | T290 | Q314 | K317 |
| 51 | Min | F270 | P272 | P273 | T274 | E298 | K301 |
| 52 | Mhu | R280 | L282 | R283 | D284 | Q309 | T312 |
| 53 | Afu | L280 | E282 | N283 | E284 | G309 | R312 |
| 54 | Htu | R290 | D292 | S293 | D294 | T319 | S322 |
| 55 | Hpa | R289 | V291 | S292 | D293 | D318 | S321 |
| 58 | Hsp | G299 | S301 | D302 | T303 | E327 | E330 |
| | (ski2-like | | | | | | |
| | helicase) | | | | | | |

The Hel308 helicase more preferably comprises a variant of one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 which comprises one or more cysteine residues and/or one or more non-natural amino acids at one or more of the positions which correspond to D274, E284, E285, S288, S615, K717 and Y720 in Hel308 Mbu (SEQ ID NO: 10). The relevant positions are shown in columns A to G in Table 4a above.

The helicase may comprise a cysteine residue at one, two, three, four, five, six or seven of the positions which correspond to D274, E284, E285, S288, S615, K717 and Y720 in Hel308 Mbu (SEQ ID NO: 10). Any combination of these positions may be substituted with cysteine. For instance, for each row of Table 4a above, the helicase of the invention may comprise a cysteine at any of the following combinations of the positions labelled A to G in that row: {A}, {B}, {C}, {D}, {G}, {E}, {F}, {A and B}, {A and C}, {A and D}, {A and G}, {A and E}, {A and F}, {B and C}, {B and D}, {B and G}, {B and E}, {B and F}, {C and D}, {C and G}, {C and E}, {C and F}, {D and G}, {D and E}, {D and F}, {G and E}, {G and F}, {E and F}, {A, B and C}, {A, B and D}, {A, B and G}, {A, B and E}, {A, B and F}, {A, C and D}, {A, C and G}, {A, C and E}, {A, C and F}, {A, D and G}, {A, D and E}, {A, D and F}, {A, G and E}, {A, G and F}, {A, E and F}, {B, C and D}, {B, C and G}, {B, C and E}, {B, C and F}, {B, D and G}, {B, D and E}, {B, D and F}, {B, G and E}, {B, G and F}, {B, E and F}, {C, D and G}, {C, D and E}, {C, D and F}, {C, G and E}, {C, G and F}, {C, E and F}, {D, G and E}, {D, G and F}, {D, E and F}, {G, E and F}, {A, B, C and D}, {A, B, C and G}, {A, B, C and E}, {A, B, C and F}, {A, B, D and G}, {A, B, D and E}, {A, B, D and F}, {A, B, G and E}, {A, B, G and F}, {A, B, E and F}, {A, C, D and G}, {A, C, D and E}, {A, C, D and F}, {A, C, G and E}, {A, C, G and F}, {A, C, E and F}, {A, D, G and E}, {A, D, G and F}, {A, D, E and F}, {A, G, E and F}, {B, C, D and G}, {B, C, D and E}, {B, C, D and F}, {B, C, G and E}, {B, C, G and F}, {B, C, E and F}, {B, D, G and E}, {B, D, G and F}, {B, D, E and F}, {B, G, E and F}, {C, D, G and E}, {C, D, G and F}, {C, D, E and F}, {C, G, E and F}, {D, G, E and F}, {A, B, C, D and G}, {A, B, C, D and E}, {A, B, C, D and F}, {A, B, C, G and E}, {A, B, C, G and F}, {A, B, C, E and F}, {A, B, D, G and E}, {A, B, D, G and F}, {A, B, D, E and F}, {A, B, G, E and F}, {A, C, D, G and E}, {A, C, D, G and F}, {A, C, D, E and F}, {A, C, G, E and F}, {A, D, G, E and F}, {B, C, D, G and E}, {B, C, D, G and F}, {B, C, D, E and F}, {B, C, G, E and F}, {B, D, G, E and F}, {C, D, G, E and F}, {A, B, C, D, G and E}, {A, B, C, D, G and F}, {A, B, C, D, E and F}, {A, B, C, G, E and F}, {A, B, D, G, E and F}, {A, C, D, G, E and F}, {B, C, D, G, E and F}, or {A, B, C, D, G, E and F}.

The helicase may comprises a non-natural amino acid, such as Faz, at one, two, three, four, five, six or seven of the positions which correspond to D274, E284, E285, S288, 5615, K717 and Y720 in Hel308 Mbu (SEQ ID NO: 10). Any combination of these positions may be substituted with a non-natural amino acid, such as Faz. For instance, for each row of Table 4a above, the helicase of the invention may comprise a non-natural amino acid, such as Faz, at any of the combinations of the positions labelled A to G above.

The helicase may comprise a combination of one or more cysteines and one or more non-natural amino acids, such as Faz, at two or more of the positions which correspond to D274, E284, E285, S288, S615, K717 and Y720 in Hel308 Mbu (SEQ ID NO: 10). Any combination of one or more cysteine residues and one or more non-natural amino acids, such as Faz, may be present at the relevant positions. For instance, for each row of Table 4a and 4b above, the helicase of the invention may comprise one or more cysteines and one or more non-natural amino acids, such as Faz, at any of the combinations of the positions labelled A to G above.

The Hel308 helicase more preferably comprises a variant of one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 which comprises one or more cysteine residues and/or one or more non-natural amino acids at one or more of the positions which correspond to D274, E284, E285, S288 and S615 in Hel308 Mbu (SEQ ID NO: 10). The relevant positions are shown in columns A to E in Table 4a above.

The helicase may comprise a cysteine residue at one, two, three, four or five, six or seven of the positions which correspond to D274, E284, E285, S288, S615, K717 and Y720 in Hel308 Mbu (SEQ ID NO: 10). Any combination of these positions may be substituted with cysteine. For instance, for each row of Table 4a above, the helicase of the invention may comprise a cysteine at any of the following combinations of the positions labelled A to E in that row: {A}, {B}, {C}, {D}, {E}, {A and B}, {A and C}, {A and D}, {A and E}, {B and C}, {B and D}, {B and E}, {C and D}, {C and E}, {D and E}, {A, B and C}, {A, B and D}, {A, B and E}, {A, C and D}, {A, C and E}, {A, D and E}, {B, C and D}, {B, C and E}, {B, D and E}, {C, D and E}, {A, B, C and D}, {A, B, C and E}, {A, B, D and E}, {A, C, D and E}, {B, C, D and E} or {A, B, C, D and E}.

The helicase may comprises a non-natural amino acid, such as Faz, at one, two, three, four or five of the positions which correspond to D274, E284, E285, S288, 5615, K717 and Y720 in Hel308 Mbu (SEQ ID NO: 10). Any combination of these positions may be substituted with a non-natural amino acid, such as Faz. For instance, for each row of Table 4a above, the helicase of the invention may comprise a non-natural amino acid, such as Faz, at any of the combinations of the positions labelled A to E above.

The helicase may comprise a combination of one or more cysteines and one or more non-natural amino acids, such as Faz, at two or more of the positions which correspond to D274, E284, E285, S288 and S615 in Hel308 Mbu (SEQ ID NO: 10). Any combination of one or more cysteine residues and one or more non-natural amino acids, such as Faz, may be present at the relevant positions. For instance, for each row of Table 4a above, the helicase of the invention may comprise one or more cysteines and one or more non-natural amino acids, such as Faz, at any of the combinations of the positions labelled A to E above.

The Hel308 helicase preferably comprises a variant of the sequence of Hel308 Mbu (i.e. SEQ ID NO: 10) which comprises one or more cysteine residues and/or one or more non-natural amino acids at D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724. The variant preferably comprises D272C, N273C, D274C, G281C, E284C, E285C, E287C, S288C, T289C, G290C, E291C, D293C, T294C, N300C, R303C, K304C, N314C, S315C, N316C, H317C, R318C, K319C, L320C, E322C, R326C, N328C, S615C, K717C, Y720C, N721C or S724C. The variant preferably comprises D272Faz, N273Faz, D274Faz, G281Faz, E284Faz, E285Faz, E287Faz, S288Faz, T289Faz, G290Faz, E291Faz, D293Faz, T294Faz, N300Faz, R303Faz, K304Faz, N314Faz, S315Faz, N316Faz, H317 Faz, R318Faz, K319Faz, L320Faz, E322Faz, R326Faz, N328Faz, S615Faz, K717Faz, Y720Faz, N721Faz or S724Faz.

The Hel308 helicase preferably comprises a variant of the sequence of Hel308 Mbu (i.e. SEQ ID NO: 10) which comprises one or more cysteine residues and/or one or more non-natural amino acids at D274, E284, E285, S288, S615, K717 and Y720. The helicase of the invention may comprise one or more cysteines, one or more non-natural amino acids, such as Faz, or a combination thereof at any of the combinations of the positions labelled A to G above.

The Hel308 helicase preferably comprises a variant of the sequence of Hel308 Mbu (i.e. SEQ ID NO: 10) which comprises one or more cysteine residues and/or one or more non-natural amino acids at one or more of D274, E284, E285, S288 and S615. For instance, for Hel308 Mbu (SEQ ID NO: 10), the helicase of the invention may comprise a cysteine or a non-natural amino acid, such as Faz, at any of the following combinations of positions: {D274}, {E284}, {E285}, {S288}, {S615}, {D274 and E284}, {D274 and E285}, {D274 and S288}, {D274 and S615}, {E284 and E285}, {E284 and S288}, {E284 and S615}, {E285 and S288}, {E285 and S615}, {S288 and S615}, {D274, E284 and E285}, {D274, E284 and S288}, {D274, E284 and S615}, {D274, E285 and S288}, {D274, E285 and S615}, {D274, S288 and S615}, {E284, E285 and S288}, {E284, E285 and S615}, {E284, S288 and S615}, {E285, S288 and S615}, {D274, E284, E285 and S288}, {D274, E284, E285 and S615}, {D274, E284, S288 and S615}, {D274, E285, S288 and S615}, {E284, E285, S288 and S615} or {D274, E284, E285, S288 and S615}.

The helicase preferably comprises a variant of SEQ ID NO: 10 which comprises (a) E284C and S615C, (b), E284Faz and S615Faz, (c) E284C and S615Faz or (d) E284Faz and S615C.

The helicase more preferably comprises the sequence shown in SEQ ID NO: 10 with E284C and S615C.

Preferred non-natural amino acids for use in the invention include, but are not limited, to 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O−2-Propyn-1-yl-L-tyrosine, 4-(Dihydroxyboryl)-L-phenylalanine, 4-[(Ethylsulfanyl)carbonyl]-L-phenylalanine, (2S)-2-amino-3-{4-[(propan-2-ylsulfanyl)carbonyl]phenyl}propanoic acid, (2S)-2-amino-3-{4-[(2-amino-3-sulfanylpropanoyl)amino]phenyl}propanoic acid, O-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, O-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2,2′-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, O-(4,5-Dimethoxy-2-nitrobenzyl)-L-serine, (2S)-2-amino-6-({[(2-nitrobenzyl)oxy]carbonyl}amino)hexanoic acid, O-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-1-naphthyl]sulfonylamino]propanoic acid, (2S)-2-amino-4-(7-hydroxy-2-oxo-2H-chromen-4-yl)butanoic acid, (2S)-3-[(6-acetylnaphthalen-2-yl)amino]-2-aminopropanoic acid, 4-(Carboxymethyl)phenylalanine, 3-Nitro-L-tyrosine, O-Sulfo-L-tyrosine, (2R)-6-Acetamido-2-ammoniohexanoate, 1-Methylhistidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N⁶-{[(2R,3R)-3-Methyl-3,4-dihydro-2H-pyrrol-2-yl]carbonyl}-L-lysine, N⁶-[(Benzyloxy)carbonyl]lysine, (2S)-2-amino-6-[(cyclopentylcarbonyl)amino]hexanoic acid, N⁶-[(Cyclopentyloxy)carbonyl]-L-lysine, (2S)-2-amino-6-{[(2R)-tetrahydrofuran-2-ylcarbonyl]amino}hexanoic acid, (2S)-2-amino-8-[(2R,3S)-3-ethynyltetrahydrofuran-2-yl]-8-oxooctanoic acid, N⁶-(tert-Butoxycarbonyl)-L-lysine, (2S)-2-Hydroxy-6-({[(2-methyl-2-propanyl)oxy]carbonyl}amino)hexanoic acid, N⁶-[(Allyloxy)carbonyl]lysine, (2S)-2-amino-6-({[(2-azidobenzyl)oxy]carbonyl}amino)hexanoic acid, N⁶-L-Prolyl-L-lysine, (2S)-2-amino-6-{[(prop-2-yn-1-yloxy)carbonyl]amino}hexanoic acid and N⁶-[(2-Azidoethoxy)carbonyl]-L-lysine.

The most preferred non-natural amino acid is 4-azido-L-phenylalanine (Faz).

As discussed above, variant of a Hel308 helicase is an enzyme that has an amino acid sequence which varies from that of the wild-type helicase and which retains polynucleotide binding activity. In the Hel308 helicases of the invention, a variant of one of SEQ ID NOs: 10, 13, 16, 19, 22, 25, 28, 29, 32, 33, 34, 37, 38, 39, 40, 41, 42, 43, 44, 45, 46, 47, 48, 49, 50, 51, 52, 53, 54, 55 and 58 may comprise additional modifications as long as it comprises one or more cysteine residues and/or one or more non-natural amino acids at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10). Suitable modifications and variants are discussed above with reference to the embodiments with two or more parts connected.

A variant may comprise the mutations in domain 5 disclosed in Woodman et al. (J. Mol. Biol. (2007)374, 1139-1144). These mutations correspond to R685A, R687A and R689A in SEQ ID NO: 10.

### Connecting Two or More Parts of the Hel308 Helicases of the Invention

The Hel308 helicases modified in the invention comprise a polynucleotide binding domain. Polynucleotide binding domains are defined above. The polynucleotide binding domain of an unmodified Hel308 helicase for use in the invention comprises an opening through which a polynucleotide can unbind from the helicase.

In a preferred embodiment, the Hel308 helicase is further modified such that two or more parts of the helicase are connected to reduce the size of an opening in the polynucleotide binding domain through which a polynucleotide can unbind from the helicase. The two or more parts may be connected in any of the ways discussed above.

### No Connection

In another embodiment, the Hel308 helicase is not modified such that two or more parts of the helicase are connected to reduce the size of an opening in the polynucleotide binding domain through which a polynucleotide can unbind from the helicase. Preferably, none of the one or more cysteines or one or more non-natural amino acids is connected to another amino acid in the helicase. Preferably, no two amino acids in the helicase are connected together via their natural or non-natural R groups.

### Construct

The invention also provides a construct comprising a helicase of the invention and an additional polynucleotide binding moiety, wherein the helicase is attached to the polynucleotide binding moiety and the construct has the ability to control the movement of a polynucleotide. The helicase is attached to the additional polynucleotide binding moiety. The construct is artificial or non-natural.

A construct of the invention is a useful tool for controlling the movement of a polynucleotide during Strand Sequencing. A construct of the invention is even less likely than a modified helicase of the invention to disengage from the polynucleotide being sequenced. The construct can provide even greater read lengths of the polynucleotide as it controls the translocation of the polynucleotide through a nanopore.

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

The construct has the ability to control the movement of a polynucleotide. This can be determined as discussed above.

A construct of the invention may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

The helicase is preferably covalently attached to the additional polynucleotide binding moiety. The helicase may be attached to the moiety at more than one, such as two or three, points.

The helicase can be covalently attached to the moiety using any method known in the art. Suitable methods are discussed above with reference to connecting the two or more parts.

The helicase and moiety may be produced separately and then attached together. The two components may be attached in any configuration. For instance, they may be attached via their terminal (i.e. amino or carboxy terminal) amino acids. Suitable configurations include, but are not limited to, the amino terminus of the moiety being attached to the carboxy terminus of the helicase and vice versa. Alternatively, the two components may be attached via amino acids within their sequences. For instance, the moiety may be attached to one or more amino acids in a loop region of the helicase. In a preferred embodiment, terminal amino acids of the moiety are attached to one or more amino acids in the loop region of a helicase.

In a preferred embodiment, the helicase is chemically attached to the moiety, for instance via one or more linker molecules as discussed above. In another preferred embodiment, the helicase is genetically fused to the moiety. A helicase is genetically fused to a moiety if the whole construct is expressed from a single polynucleotide sequence. The coding sequences of the helicase and moiety may be combined in any way to form a single polynucleotide sequence encoding the construct. Genetic fusion of a pore to a nucleic acid binding protein is discussed in International Application No. PCT/GB09/001679 (published as WO 2010/004265).

The helicase and moiety may be genetically fused in any configuration. The helicase and moiety may be fused via their terminal amino acids. For instance, the amino terminus of the moiety may be fused to the carboxy terminus of the helicase and vice versa. The amino acid sequence of the moiety is preferably added in frame into the amino acid sequence of the helicase. In other words, the moiety is preferably inserted within the sequence of the helicase. In such embodiments, the helicase and moiety are typically attached at two points, i.e. via the amino and carboxy terminal amino acids of the moiety. If the moiety is inserted within the sequence of the helicase, it is preferred that the amino and carboxy terminal amino acids of the moiety are in close proximity and are each attached to adjacent amino acids in the sequence of the helicase or variant thereof. In a preferred embodiment, the moiety is inserted into a loop region of the helicase.

The construct retains the ability of the helicase to control the movement of a polynucleotide. This ability of the helicase is typically provided by its three dimensional structure that is typically provided by its β-strands and α-helices. The α-helices and β-strands are typically connected by loop regions. In order to avoid affecting the ability of the helicase to control the movement of a polynucleotide, the moiety is preferably genetically fused to either end of the helicase or inserted into a surface-exposed loop region of the helicase. The loop regions of specific helicases can be identified using methods known in the art. In the Hel308 embodiments of the invention, the moiety is preferably not genetically fused to any of the α-helixes.

The helicase may be attached directly to the moiety. The helicase is preferably attached to the moiety using one or more, such as two or three, linkers as discussed above. The one or more linkers may be designed to constrain the mobility of the moiety. The helicase and/or the moiety may be modified to facilitate attachment of the one or more linker as discussed above.

Cleavable linkers can be used as an aid to separation of constructs from non-attached components and can be used to further control the synthesis reaction. For example, a hetero-bifunctional linker may react with the helicase, but not the moiety. If the free end of the linker can be used to bind the helicase protein to a surface, the unreacted helicases from the first reaction can be removed from the mixture. Subsequently, the linker can be cleaved to expose a group that reacts with the moiety. In addition, by following this sequence of linkage reactions, conditions may be optimised first for the reaction to the helicase, then for the reaction to the moiety after cleavage of the linker. The second reaction would also be much more directed towards the correct site of reaction with the moiety because the linker would be confined to the region to which it is already attached.

The helicase may be covalently attached to the bifunctional crosslinker before the helicase/crosslinker complex is covalently attached to the moiety. Alternatively, the moiety may be covalently attached to the bifunctional crosslinker before the bifunctional crosslinker/moiety complex is attached to the helicase. The helicase and moiety may be covalently attached to the chemical crosslinker at the same time.

Preferred methods of attaching the helicase to the moiety are cysteine linkage and Faz linkage as described above. In a preferred embodiment, a reactive cysteine is presented on a peptide linker that is genetically attached to the moiety. This means that additional modifications will not necessarily be needed to remove other accessible cysteine residues from the moiety.

Cross-linkage of helicases or moieties to themselves may be prevented by keeping the concentration of linker in a vast excess of the helicase and/or moiety. Alternatively, a “lock and key” arrangement may be used in which two linkers are used. Only one end of each linker may react together to form a longer linker and the other ends of the linker each react with a different part of the construct (i.e. helicase or moiety). This is discussed in more detail below.

The site of attachment is selected such that, when the construct is contacted with a polynucleotide, both the helicase and the moiety can bind to the polynucleotide and control its movement.

Attachment can be facilitated using the polynucleotide binding activities of the helicase and the moiety. For instance, complementary polynucleotides can be used to bring the helicase and moiety together as they hybridize. The helicase can be bound to one polynucleotide and the moiety can be bound to the complementary polynucleotide. The two polynucleotides can then be allowed to hybridise to each other. This will bring the helicase into close contact with the moiety, making the linking reaction more efficient. This is especially helpful for attaching two or more helicases in the correct orientation for controlling movement of a target polynucleotide. An example of complementary polynucleotides that may be used are shown below.

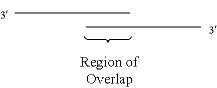

For helicase-Phi29 constructs the DNA below could be used.

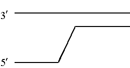

Tags can be added to the construct to make purification of the construct easier. These tags can then be chemically or enzymatically cleaved off, if their removal is necessary. Fluorophores or chromophores can also be included, and these could also be cleavable.

A simple way to purify the construct is to include a different purification tag on each protein (i.e. the helicase and the moiety), such as a hexa-His-tag and a Strep-tag®. If the two proteins are different from one another, this method is particularly useful. The use of two tags enables only the species with both tags to be purified easily.

If the two proteins do not have two different tags, other methods may be used. For instance, proteins with free surface cysteines or proteins with linkers attached that have not reacted to form a construct could be removed, for instance using an iodoacetamide resin for maleimide linkers.

Constructs of the invention can also be purified from unreacted proteins on the basis of a different DNA processivity property. In particular, a construct of the invention can be purified from unreacted proteins on the basis of an increased affinity for a polynucleotide, a reduced likelihood of disengaging from a polynucleotide once bound and/or an increased read length of a polynucleotide as it controls the translocation of the polynucleotide through a nanopore

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

### Polynucleotide Binding Moiety

The constructs of the invention comprise a polynucleotide binding moiety. A polynucleotide binding moiety is a polypeptide that is capable of binding to a polynucleotide. The moiety is preferably capable of specific binding to a defined polynucleotide sequence. In other words, the moiety preferably binds to a specific polynucleotide sequence, but displays at least 10 fold less binding to different sequences or more preferably at least 100 fold less binding to different sequences or most preferably at least 1000 fold less binding to different sequences. The different sequence may be a random sequence. In some embodiments, the moiety binds to a specific polynucleotide sequence, but binding to different sequences cannot be measured. Moieties that bind to specific sequences can be used to design constructs that are targeted to such sequences.

The moiety typically interacts with and modifies at least one property of a polynucleotide. The moiety may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated. One or more nucleotides in the target polynucleotide may be damaged. For instance, the polynucleotide may comprise a pyrimidine dimer. Such dimers are typically associated with damage by ultraviolet light and are the primary cause of skin melanomas. One or more nucleotides in the target polynucleotide may be modified, for instance with a label or a tag. Suitable labels are described above. The target polynucleotide may comprise one or more spacers.

A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP) and deoxycytidine monophosphate (dCMP). The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

It is preferred that the tertiary structure of the moiety is known. Knowledge of the three dimensional structure of the moiety allows modifications to be made to the moiety to facilitate its function in the construct of the invention.

The moiety may be any size and have any structure. For instance, the moiety may be an oligomer, such as a dimer or trimer. The moiety is preferably a small, globular polypeptide formed from one monomer. Such moieties are easy to handle and are less likely to interfere with the ability of the helicase to control the movement of the polynucleotide, particularly if fused to or inserted into the sequence of the helicase.

The amino and carboxy terminii of the moiety are preferably in close proximity. The amino and carboxy terminii of the moiety are more preferably presented on same face of the moiety. Such embodiments facilitate insertion of the moiety into the sequence of the helicase. For instance, if the amino and carboxy terminii of the moiety are in close proximity, each can be attached by genetic fusion to adjacent amino acids in the sequence of the helicase.

It is also preferred that the location and function of the active site of the moiety is known. This prevents modifications being made to the active site that abolish the activity of the moiety. It also allows the moiety to be attached to the helicase so that the moiety binds to the polynucleotide and controls its movement. Knowledge of the way in which a moiety may bind to and orient polynucleotides also allows an effective construct to be designed.

The constructs of the invention are useful in Strand Sequencing. The moiety preferably binds the polynucleotide in a buffer background which is compatible with Strand Sequencing and the discrimination of the nucleotides. The moiety preferably has at least residual activity in a salt concentration well above the normal physiological level, such as from 100 mM to 2M. The moiety is more preferably modified to increase its activity at high salt concentrations. The moiety may also be modified to improve its processivity, stability and shelf life.

Suitable modifications can be determined from the characterisation of polynucleotide binding moieties from extremphiles such as halophilic, moderately halophilic bacteria, thermophilic and moderately thermophilic organisms, as well as directed evolution approaches to altering the salt tolerance, stability and temperature dependence of mesophilic or thermophilic exonucleases.

The polynucleotide binding moiety preferably comprises one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

The helix-hairpin-helix (HhH) domains are polypeptide motifs that bind DNA in a sequence non-specific manner. They have been shown to confer salt stability and processivity when fused to polymerases, as well as increasing their thermal stability. Suitable domains include domain H (residues 696-751) and domain HI (residues 696-802) from Topoisomerase V from Methanopyrus kandleri (SEQ ID NO: 129). As discussed below, the polynucleotide binding moiety may be domains H-L of SEQ ID NO: 129 as shown in SEQ ID NO: 130. Topoisomerase V from Methanopyrus kandleri is an example of a double-stranded binding protein as discussed below.

The HhH domain preferably comprises the sequence shown in SEQ ID NO: 94 or 107 or 108 or a variant thereof. This domain increases the processivity and the salt tolerance of a helicase when used in a construct of the invention. A variant of SEQ ID NO: 94 or 107 or 108 is a protein that has an amino acid sequence which varies from that of SEQ ID NO: 94 or 107 or 108 and which retains polynucleotide binding activity. This can be measured as described above. A variant typically has at least 50% homology to SEQ ID NO: 94 or 107 or 108 based on amino acid identity over its entire sequence (or any of the % homologies discussed above in relation to helicases) and retains polynucleotide binding activity. A variant may differ from SEQ ID NO: 94 or 107 or 108 in any of the ways discussed above in relation to helicases or below in relation to pores. A variant preferably comprises one or more substituted cysteine residues and/or one or more substituted Faz residues to facilitate attachment to the helicase as discussed above.

SSBs bind single stranded DNA with high affinity in a sequence non-specific manner. They exist in all domains of life in a variety of forms and bind DNA either as monomers or multimers. Using amino acid sequence alignment and logarithms (such as Hidden Markov models) SSBs can be classified according to their sequence homology. The Pfam family, PF00436, includes proteins that all show sequence similarity to known SSBs. This group of SSBs can then be further classified according to the Structural Classification of Proteins (SCOP). SSBs fall into the following lineage: Class; All beta proteins, Fold; OB-fold, Superfamily: Nucleic acid-binding proteins, Family; Single strand DNA-binding domain, SSB. Within this family SSBs can be classified according to subfamilies, with several type species often characterised within each subfamily.

The SSB may be from a eukaryote, such as from humans, mice, rats, fungi, protozoa or plants, from a prokaryote, such as bacteria and archaea, or from a virus.

Eukaryotic SSBs are known as replication protein A (RPAs). In most cases, they are hetero-trimers formed of different size units. Some of the larger units (e.g. RPA70 of Saccharomyces cerevisiae) are stable and bind ssDNA in monomeric form.

Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). The SSBs from archaeal genomes are considered to be related with eukaryotic RPAs. Few of them, such as the SSB encoded by the crenarchaeote Sulfolobus solfataricus, are homo-tetramers. The SSBs from most other species are closer related to the replication proteins from eukaryotes and are referred to as RPAs. In some of these species they have been shown to be monomeric (Methanococcus jannaschii and Methanothermobacter thermoautotrophicum). Still, other species of Archaea, including Archaeoglobus fulgidus and Methanococcoides burtonii, appear to each contain two open reading frames with sequence similarity to RPAs. There is no evidence at protein level and no published data regarding their DNA binding capabilities or oligomeric state. However, the presence of two oligonucleotide/oligosaccharide (OB) folds in each of these genes (three OB folds in the case of one of the M. burtonii ORFs) suggests that they also bind single stranded DNA.

Viral SSBs bind DNA as monomers. This, as well as their relatively small size renders them amenable to genetic fusion to other proteins, for instance via a flexible peptide linker. Alternatively, the SSBs can be expressed separately and attached to other proteins by chemical methods (e.g. cysteines, unnatural amino-acids). This is discussed in more detail below.

The SSB is preferably either (i) an SSB comprising a carboxy-terminal (C-terminal) region which does not have a net negative charge or (ii) a modified SSB comprising one or more modifications in its C-terminal region which decreases the net negative charge of the C-terminal region. Such SSBs do not block the transmembrane pore and therefore allow characterization of the target polynucleotide.

Examples of SSBs comprising a C-terminal region which does not have a net negative charge include, but are not limited to, the human mitochondrial SSB (HsmtSSB; SEQ ID NO: 118, the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 119) or a variant of any of those proteins. A variant is a protein that has an amino acid sequence which varies from that of the wild-type protein and which retains single stranded polynucleotide binding activity. Polynucleotide binding activity can be determined using methods known in the art (and as described above). For instance, the ability of a variant to bind a single stranded polynucleotide can be determined as described in the Examples.

A variant of SEQ ID NO 118 or 119 typically has at least 50% homology to SEQ ID NO: 118 or 119 based on amino acid identity over its entire sequence (or any of the % homologies discussed above in relation to helicases) and retains single stranded polynucleotide binding activity. A variant may differ from SEQ ID NO: 118 or 119 in any of the ways discussed above in relation to helicases. In particular, a variant may have one or more conservative substitutions as shown in Tables 8 and 9.

Examples of SSBs which require one or more modifications in their C-terminal region to decrease the net negative charge include, but are not limited to, the SSB of E. coli (EcoSSB; SEQ ID NO: 120, the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDCl3 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 121), the SSB from RB69 (gp32; SEQ ID NO: 95), the SSB from T7 (gp2.5; SEQ ID NO: 96) or a variant of any of these proteins. Hence, the SSB used in the method of the invention may be derived from any of these proteins.

In addition to the one or more modifications in the C-terminal region, the SSB used in the method may include additional modifications which are outside the C-terminal region or do not decrease the net negative charge of the C-terminal region. In other words, the SSB used in the method of the invention is derived from a variant of a wild-type protein. A variant is a protein that has an amino acid sequence which varies from that of the wild-type protein and which retains single stranded polynucleotide binding activity. Polynucleotide binding activity can be determined as discussed above.

The SSB used in the invention may be derived from a variant of SEQ ID NO: 95, 96, 120 or 121. In other words, a variant of SEQ ID NO: 95, 96, 120 or 121 may be used as the starting point for the SSB used in the invention, but the SSB actually used further includes one or more modifications in its C-terminal region which decreases the net negative charge of the C-terminal region. A variant of SEQ ID NO: 95, 96, 120 or 121 typically has at least 50% homology to SEQ ID NO: 95, 96, 120 or 121 based on amino acid identity over its entire sequence (or any of the % homologies discussed above in relation to helicases) and retains single stranded polynucleotide binding activity. A variant may differ from SEQ ID NO: 95, 96, 120 or 121 in any of the ways discussed above in relation to helicases. In particular, a variant may have one or more conservative substitutions as shown in Tables 8 and 9.

It is straightforward to identify the C-terminal region of the SSB in accordance with normal protein N to C nomenclature. The C-terminal region of the SSB is preferably about the last third of the SSB at the C-terminal end, such as the last third of the SSB at the C-terminal end. The C-terminal region of the SSB is more preferably about the last quarter, fifth or eighth of the SSB at the C-terminal end, such as the last quarter, fifth or eighth of the SSB at the C-terminal end. The last third, quarter, fifth or eighth of the SSB may be measured in terms of numbers of amino acids or in terms of actual length of the primary structure of the SSB protein. The length of the various amino acids in the N to C direction are known in the art.

The C-terminal region is preferably from about the last 10 to about the last 60 amino acids of the C-terminal end of the SSB. The C-terminal region is more preferably about the last 15, about the last 20, about the last 25, about the last 30, about the last 35, about the last 40, about the last 45, about the last 50 or about the last 55 amino acids of the C-terminal end of the SSB.

The C-terminal region typically comprises a glycine and/or proline rich region. This proline/glycine rich region gives the C-terminal region flexibility and can be used to identify the C-terminal region.

Suitable modifications for decreasing the net negative charge are disclosed in U.S. Provisional Application No. 61/673,457 (filed 19 Jul. 2012), U.S. Provisional Application No. 61/774,688 (filed 8 Mar. 2013) and the International application being filed concurrently with this application (Oxford Nanopore Ref: ONT IP 035). The SSB may be any of the SSBs disclosed in the US Provisional Applications and International application.

The modified SSB most preferably comprises a sequence selected from those shown in SEQ ID NOs: 103, 104, 122 to 125.

Double-stranded binding proteins bind double stranded DNA with high affinity. Suitable double-stranded binding proteins include, but are not limited to Mutator S (MutS; NCBI Reference Sequence: NP_417213.1; SEQ ID NO: 140), Sso7d (Sufolobus solfataricus P2; NCBI Reference Sequence: NP_343889.1; SEQ ID NO: 141; Nucleic Acids Research, 2004, Vol 32, No. 3, 1197-1207), Sso10b1 (NCBI Reference Sequence: NP_342446.1; SEQ ID NO: 142), Sso10b2 (NCBI Reference Sequence: NP_342448.1; SEQ ID NO: 143), Tryptophan repressor (Trp repressor; NCBI Reference Sequence: NP_291006.1; SEQ ID NO: 144), Lambda repressor (NCBI Reference Sequence: NP_040628.1; SEQ ID NO: 145), Cren7 (NCBI Reference Sequence: NP_342459.1; SEQ ID NO: 146), major histone classes H1/H5, H2A, H2B, H3 and H4 (NCBI Reference Sequence: NP_066403.2, SEQ ID NO: 147), dsbA (NCBI Reference Sequence: NP_049858.1; SEQ ID NO: 148), Rad51 (NCBI Reference Sequence: NP_002866.2; SEQ ID NO: 149), sliding clamps and Topoisomerase V Mka (SEQ ID NO: 129) or a variant of any of these proteins. A variant of SEQ ID NO: 129, 140, 141, 142, 143, 144, 145, 146, 147, 148 or 149 typically has at least 50% homology to SEQ ID NO: 129, 140, 141, 142, 143, 144, 145, 146, 147, 148 or 149 based on amino acid identity over its entire sequence (or any of the % homologies discussed above in relation to helicases) and retains single stranded polynucleotide binding activity. A variant may differ from SEQ ID NO: 129, 140, 141, 142, 143, 144, 145, 146, 147, 148 or 149 in any of the ways discussed above in relation to helicases. In particular, a variant may have one or more conservative substitutions as shown in Tables 8 and 9. Most polymerases achieve processivity by interacting with sliding clamps. In general, these are multimeric proteins (homo-dimers or homo-trimers) that encircle dsDNA. These sliding clamps require accessory proteins (clamp loaders) to assemble them around the DNA helix in an ATP-dependent process. They also do not contact DNA directly, acting as a topological tether. As sliding clamps interact with their cognate polymerases in a specific manner via a polymerase domain, this fragment could be fused to the helicase in order to incite recruitment of helicases onto the sliding clamp. This interaction could be further stabilized by the generation of a covalent bond (introduction of cysteines or unnatural amino-acids).

Related to DNA sliding clamps, processivity factors are viral proteins that anchor their cognate polymerases to DNA, leading to a dramatic increase in the length of the fragments generated. They can be monomeric (as is the case for UL42 from Herpes simplex virus 1) or multimeric (UL44 from Cytomegalovirus is a dimer), they do not form closed rings around the DNA strand and they contact DNA directly. UL42 has been shown to increase processivity without reducing the rate of its corresponding polymerase, suggesting that it interacts with DNA in a different mode to SSBs. The UL42 preferably comprises the sequence shown in SEQ ID NO: 97 or SEQ ID NO: 102 or a variant thereof. A variant of SEQ ID NO: 97 or 102 is a protein that has an amino acid sequence which varies from that of SEQ ID NO: 97 or 102 and which retains polynucleotide binding activity. This can be measured as described above. A variant typically has at least 50% homology to SEQ ID NO: 97 or 102 based on amino acid identity over its entire sequence (or any of the % homologies discussed above in relation to helicases) and retains polynucleotide binding activity. A variant may differ from SEQ ID NO: 97 or SEQ ID NO: 102 in any of the ways discussed above in relation to helicases or below in relation to pores. A variant preferably comprises one or more substituted cysteine residues and/or one or more substituted Faz residues to facilitate attachment to the helicase as discussed above.

Attaching UL42 to a helicase could be done via genetic fusion or chemical attachment (cysteines, unnatural amino-acids). As the polymerase polypeptide that binds UL42 is visible in the crystal structure, these 35 amino acids (residues 1200-1235) could be fused onto the C-terminus of the helicase and the natural affinity between this polypeptide and the processivity factor used to form a complex. The interaction could be stabilized by introducing a covalent interaction (cysteines or unnatural amino-acids). One option is to utilize a natural UL42 cysteine (C300) that is located close to the polypeptide interaction site and introduce a point mutation into the polymerase polypeptide (e.g. L1234C).

A reported method of increasing polymerase processivity is by exploiting the interaction between E. coli thioredoxin (Trx) and the thioredoxin binding domain (TBD) of bacteriophage T7 DNA polymerase (residues 258-333). The binding of Trx to TBD causes the polypeptide to change conformation to one that binds DNA. TBD is believed to clamp down onto a DNA strand and limit the polymerase off-rate, thus increasing processivity. Chimeric polymerases have been made by transferring TBD onto a non-processive polymerase, resulting in 1000 fold increase in polymerised fragment length. There were no attempts to attach TBD to any other class of proteins, but a covalent link between TBD and Trx was engineered and can be used to stabilise the interaction.

Some helicases use accessory proteins in-vivo to achieve processivity (e.g. cisA from phage ΦDx174 and geneII protein from phage M13 for E. coli Rep helicase). Some of these proteins have been shown to interact with more than one helicase (e.g. MutL acts on both UvrD and Rep, though not to the same extent). These proteins have intrinsic DNA binding capabilities, some of them recognizing a specific DNA sequence. The ability of some of these accessory proteins to covalently attach themselves to a specific DNA sequence could also be used to create a set starting point for the helicase activity.

The proteins that protect the ends of chromosomes bind to telomeric ssDNA sequences in a highly specific manner. This ability could either be exploited as is or by using point mutations to abolish the sequence specificity.

Small DNA binding motifs (such as helix-turn-helix) recognize specific DNA sequences. In the case of the bacteriophage 434 repressor, a 62 residue fragment was engineered and shown to retain DNA binding abilities and specificity.

An abundant motif in eukaryotic proteins, zinc fingers consist of around 30 amino-acids that bind DNA in a specific manner. Typically each zinc finger recognizes only three DNA bases, but multiple fingers can be linked to obtain recognition of a longer sequence.

Proliferating cell nuclear antigens (PCNAs) form a very tight clamp (doughnut) which slides up and down the dsDNA or ssDNA. The PCNA from crenarchaeota is unique in being a hetero-trimer so it is possible to functionalise one subunit and retain activity. Its subunits are shown in SEQ ID NOs: 98, 99 and 100. The PCNA is preferably a trimer comprising the sequences shown in SEQ ID NOs: 98, 99 and 100 or variants thereof. PCNA sliding clamp (NCBI Reference Sequence: ZP_06863050.1; SEQ ID NO: 150) forms a dimer. The PCNA is preferably a dimer comprising SEQ ID NO: 150 or a variant thereof. A variant is a protein that has an amino acid sequence which varies from that of SEQ ID NO: 98, 99, 100 or 150 and which retains polynucleotide binding activity. This can be measured as described above. A variant is typically a trimer comprising sequences that have at least 50% homology to SEQ ID NOs: 98, 99 and 100 or a dimer comprising sequences that have at least 50% homology to SEQ ID NO: 150 based on amino acid identity over each entire sequence (or any of the % homologies discussed above in relation to helicases) and which retains polynucleotide binding activity. A variant may comprise sequences which differ from SEQ ID NO: 98, 99, 100 or 150 in any of the ways discussed above in relation to helicases or below in relation to pores. A variant preferably comprises one or more substituted cysteine residues and/or one or more substituted Faz residues to facilitate attachment to the helicase as discussed above. In a preferred embodiment, subunits 1 and 2 of the PCNA from crenarchaeota (i.e. SEQ ID NOs: 98 and 99 or variants thereof) are attached, such as genetically fused, and the resulting protein is attached to a helicase to form a construct of the invention. During use of the construct, subunit 3 (i.e. SEQ ID NO: 100 or a variant thereof) may be added to complete the PCNA clamp (or doughnut) once the construct has bound the polynucleotide. In a preferred embodiment, one monomer of the PCNA sliding clamp (i.e. SEQ ID NO: 150 or a variant thereof) is attached, such as genetically fused, to a helicase to form a construct of the invention. During use of the construct, the second monomer (i.e. SEQ ID NO: 150 or a variant thereof) may be added to complete the PCNA clamp (or doughnut) once the construct has bound the polynucleotide.

The polynucleotide binding motif may be selected from any of those shown in Table 5 below.

TABLE 5
Suitable polynucleotide binding motifs

| | | | | | | | MW | |
|---|---|---|---|---|---|---|---|---|
| No. | Name | Class | Organism | Structure | Sequence | Functional form | (Da) | Notes |
| 1 | SSBEco | ssb | Escherichia coli | 1QVC, | P0AGE0 | homo-tetramer | 18975 | |
| | | | | 1EYG | | | | |
| 2 | SSBBhe | ssb | Bartonella | 3LGJ, | Q6G302 | homo-tetramer | 16737 | structure only |
| | | | henselae | 3PGZ | | | | |
| 3 | SSBCbu | ssb | Coxiella burnetii | 3TQY | Q83EP4 | homo-tetramer | 17437 | structure only |
| 4 | SSBTma | ssb | Thermathoga | 1Z9F | Q9WZ73 | homo-dimer | 16298 | small, |
| | | | maritima | | | | | thermostable, |
| | | | | | | | | salt independent |
| | | | | | | | | DNA binding |
| 5 | SSBHpy | ssb | Helicobacter | 2VW9 | O25841 | homo-tetramer | 20143 | |
| | | | pylori | | | | | |
| 6 | SSBDra | ssb | Deinococcus | 1SE8 | Q9RY51 | homo-dimer | 32722 | |
| | | | radiodurans | | | | | |
| 7 | SSBTaq | ssb | Thermus | 2FXQ | Q9KH06 | homo-dimer | 30026 | |
| | | | aquaticus | | | | | |
| 8 | SSBMsm | ssb | Mycobacterium | 3A5U, | Q9AFI5 | homo-tetramer | 17401 | tetramer more |
| | | | smegmatis | 1X3E | | | | stable than |
| | | | | | | | | E. coli, binding |
| | | | | | | | | less salt |
| | | | | | | | | dependent |
| 9 | SSBSso | ssb/RPA | Sulfolobus | 1O7I | Q97W73 | homo-tetramer | 16138 | similarities with |
| | | | solfataricus | | | | | RPA |
| 10 | SSBMHsmt | ssb | Homo sapiens | 3ULL | Q04837 | homo-tetramer | 17260 | |
| 11 | SSBMle | ssb | Mycobacterium | 3AFP | P46390 | homo-tetramer | 17701 | |
| | | | leprae | | | | | |
| 12 | gp32T4 | ssb | Bacteriohage T4 | 1GPC | P03695 | monomer | 33506 | Homo-dimer in |
| | | | | | | | | the absence of |
| | | | | | | | | DNA, monomer |
| | | | | | | | | when binding |
| | | | | | | | | DNA. |
| 13 | gp32RB69 | ssb | Bacteriophage | 2A1K | Q7Y265 | monomer | 33118 | |
| | | | RB69 | | | | | |
| 14 | gp2.5T7 | ssb | Bacteriohage T7 | 1JE5 | P03696 | monomer | 25694 | |
| 15 | UL42 | pro- | Herpes virus 1 | 1DML | P10226 | monomer | 51159 | binds ssDNA |
| | | cessivity | | | | | | dsDNA, |
| | | factor | | | | | | structure shows |
| | | | | | | | | link with |
| | | | | | | | | polymerase |
| 16 | UL44 | pro- | Herpes virus 5 | 1YYP | P16790 | homo-dimer | 46233 | forms C shaped |
| | | cessivity | (cytomegalovirus) | | | | | clamp on DNA |
| | | factor | | | | | | |
| 17 | pf8 | pro- | KSHV | 3I2M | Q77ZG5 | homo-dimer | 42378 | |
| | | cessivity | | | | | | |
| | | factor | | | | | | |
| 18 | RPAMja | RPA | Methanococcus | 3DM3 | Q58559 | monomer | 73842 | contains 4 OB |
| | | | jannaschii | | | | | folds. Structure |
| | | | | | | | | of fragment |
| 19 | RPAMma | RPA | Methanococcus | 3E0E, | Q6LYF9 | monomer | 71388 | Core domain |
| | | | maripaludis | 2K5V | | | | structure |
| 20 | RPAMth | RPA | Methanothermo- | | | monomer | 120000 | Shown to |
| | | | bacter | | | | | interact directly |
| | | | thermo- | | | | | with Hel308. |
| | | | autotrophicus | | | | | Sequence from |
| | | | | | | | | paper. |
| 21 | RPA70Sce | RPA | Saccharomyces | 1YNX | P22336 | hetero-trimer | 70348 | unit has two OB |
| | | | cerevisiae | | | | | folds and binds |
| | | | | | | | | DNA |
| 22 | RPAMbul | RPA | Methanococcoides | | Q12V72 | ? | 41227 | three OB folds |
| | | | burtonii | | | | | identified |
| 23 | RPAMbu2 | RPA | Methanococcoides | | Q12W96 | ? | 47082 | two OB folds |
| | | | burtonii | | | | | identified |
| 24 | RPA70Hsa | RPA | Homo sapiens | 1JMC | P27694 | hetero-trimer | 68138 | |
| 25 | RPA14Hsa | RPA | Homo sapiens | 3KDF | P35244 | hetero-trimer | 13569 | in complex with |
| | | | | | | | | RPA32 |
| 26 | gp45T4 | sliding | Bacteriophage | 1CZD | P04525 | homo-trimer | 24858 | ring shape |
| | | clamp | T4 | | | | | threads DNA |
| 27 | BetaEco | sliding | E. coli | 3BEP | P0A988 | homo-dimer | 40587 | ring shape |
| | | clamp | | | | | | threads DNA, |
| | | | | | | | | may bind ssDNA |
| | | | | | | | | in poket |
| 28 | PCNASce | sliding | Saccharomyces | 1PLQ, | P15873 | homo-dimer | 28916 | ring shape |
| | | clamp | cerevisiae | 3K4X | | | | threads DNA |
| 29 | PCNATko | sliding | Thermococcus | 3LX1 | Q5JF32 | homo-dimer | 28239 | |
| | | clamp | kodakaraensis | | | | | |
| 30 | PCNAHvo | sliding | Haloferax | 3IFV | D0VWY8 | homo-dimer | 26672 | |
| | | clamp | volcanii | | | | | |
| 31 | PCNAPfu | sliding | Pyrococcus | 1GE8 | O73947 | homo-dimer | 28005 | |
| | | clamp | furiosus | | | | | |
| 32 | PCNAMbu | sliding | Methanococcoides | | Q12U18 | homo-dimer | 27121 | Inferred from |
| | | clamp | burtonii | | | | | homology |
| 33 | BetaMtu | sliding | Mycobacterium | 3P16 | Q50790 | homo-dimer | 42113 | |
| | | clamp | tuberculosis | | | | | |
| 34 | BetaTma | sliding | Thermotoga | 1VPK | Q9WYA0 | homo-dimer | 40948 | |
| | | clamp | maritima | | | | | |
| 35 | BetaSpy | sliding | Streptococcus | 2AVT | Q9EVR1 | homo-dimer | 41867 | |
| | | clamp | pyrogenes | | | | | |
| 36 | gp45RB69 | sliding | Bacteriophage | 1B77 | O80164 | homo-trimer | 25111 | Structure shows |
| | | clamp | RB69 | | | | | interaction with |
| | | | | | | | | polypeptide fom |
| | | | | | | | | polymerase |
| 37 | p55Hsa | DNA | Homo sapiens | 2G4C, | Q9UHN | monomer | 54911 | interacts with |
| | | binding | (mitochondrial) | 3IKL, | | | | specific |
| | | protein | | 3IKM | | | | polymerase |
| | | | | | | | | domain |
| 38 | p55Dme | DNA | Drosophylla | | Q9VJV8 | monomer | 41027 | associates with |
| | | binding | melanogaster | | | | | polymerase |
| | | protein | | | | | | Gamma |
| | | | | | | | | conferring salt |
| | | | | | | | | tolerance, |
| | | | | | | | | processivity and |
| | | | | | | | | increased activity |
| 39 | p55Xla | DNA | Xenopus laevis | | Q9W6G7 | monomer | 52283 | |
| | | binding | | | | | | |
| | | protein | | | | | | |
| 40 | RepDSau | replica- | Staphylococcus | | P08115 | homo-dimer | 37874 | increases |
| | | tion | aureus | | | | | processivity of |
| | | initiation | | | | | | PcrA, covalently |
| | | protein | | | | | | and specifically |
| | | | | | | | | links DNA |
| 41 | G2P | replica | Enterobacteria | | P69546 | monomer | 46168 | increases |
| | | tion | phage l | | | | | processivity of |
| | | initiation | | | | | | Rep, covalently |
| | | protein | | | | | | and specifically |
| | | | | | | | | links DNA |
| 42 | MutLEco | mismatch | Escherichia coli | 1BKN, | P23367 | homo-dimer | 67924 | increases |
| | | repair | | 1B62, | | | | processivity of |
| | | protein | | 1B63 | | | | UvrD (and Rep) |
| 43 | KuMtu | DNA | Mycobacterium | | O05866 | homo-dimer | 30904 | increases |
| | | repair | tuberculosis | | | | | processivity of |
| | | protein | | | | | | UvrD1. Structure |
| | | | | | | | | available for |
| | | | | | | | | human Ku |
| 44 | OnTEBP | telomere | Oxytricha | 1OTC | P29549 | hetero-dimer | 56082 | Specific biding |
| | | binding | nova-Alpha | | | | | to 3′ end |
| | | protein | | | | | | T4G4T4G4. |
| | | | | | | | | Alpha subunit |
| | | | | | | | | may be enough |
| | | | Oxytricha | | P16458 | | 41446 | |
| | | | nova-Beta | | | | | |
| 45 | EcrTEBP | telomere | Euplotes crassus | | Q06183 | monomer | 53360 | Homolog to |
| | | binding | | | | | | OnTEBP with no |
| | | protein | | | | | | Beta subunit in |
| | | | | | | | | genome |
| 46 | TteTEBP | telomere | Tetrachymena | | Q23FB9 | hetero-dimer | 53073 | Homolog to |
| | | binding | termophila Alpha | | | | | OnTEBP-Alpha |
| | | protein | | | | | | |
| | | | Tetrachymena | | Q23FH0 | | 54757 | May be homolog |
| | | | termophila Beta | | | | | to OnTEBP Beta |
| 47 | pot1Spo | telomere | Schizosaccharo- | | O13988 | monomer | 64111 | related to TEBP |
| | | binding | myces pombe | | | | | |
| | | proteins | | | | | | |
| 48 | Cdc13pSce | telomere | Saccharomyces | | C7GSV7 | monomer | 104936 | specific binding |
| | | binding | cerevisiae | | | | | to telomeric |
| | | proteins | | | | | | DNA |
| 49 | C1 | repressor | Bacteriophage | | P16117 | homo-dimer | 10426 | binds DNA |
| | | | 434 | | | | | specifically as |
| | | | | | | | | homo-dimer |
| 50 | LexA | repressor | Escherichia coli | 1LEB | P0A7C2 | homo-dimer | 22358 | binds DNA |
| | | | | | | | | specifically as |
| | | | | | | | | homo-dimer |

The polynucleotide binding moiety is preferably derived from a polynucleotide binding enzyme. A polynucleotide binding enzyme is a polypeptide that is capable of binding to a polynucleotide and interacting with and modifying at least one property of the polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide binding moiety does not need to display enzymatic activity as long as it is capable of binding the polynucleotide and controlling its movement. For instance, the moiety may be derived from an enzyme that has been modified to remove its enzymatic activity or may be used under conditions which prevent it from acting as an enzyme.

The polynucleotide binding moiety is preferably derived from a nucleolytic enzyme. The enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are exonucleases, polymerases, helicases and topoisomerases, such as gyrases. Suitable exonucleases include, but are not limited to, exonuclease I from E. coli, exonuclease III enzyme from E. coli, RecJ from T. thermophilus and bacteriophage lambda exonuclease and variants thereof.

The polymerase is preferably a member of any of the Moiety Classification (EC) groups 2.7.7.6, 2.7.7.7, 2.7.7.19, 2.7.7.48 and 2.7.7.49. The polymerase is preferably a DNA-dependent DNA polymerase, an RNA-dependent DNA polymerase, a DNA-dependent RNA polymerase or an RNA-dependent RNA polymerase. The polynucleotide binding moiety is preferably derived from Phi29 DNA polymerase (SEQ ID NO: 101). The moiety may comprise the sequence shown in SEQ ID NO: 101 or a variant thereof. A variant of SEQ ID NO: 101 is an enzyme that has an amino acid sequence which varies from that of SEQ ID NO: 101 and which retains polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NO: 101, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 101 over the entire sequence. There may be at least 80%, for example at least 85%, 90% or 95%, amino acid identity over a stretch of 200 or more, for example 230, 250, 270 or 280 or more, contiguous amino acids (“hard homology”). Homology is determined as described below. The variant may differ from the wild-type sequence in any of the ways discussed below with reference to SEQ ID NOs: 2 and 4.

The helicase may be any of those discussed above. Helicase dimers and multimers are discussed in detail below. The polynucleotide binding moiety may be a polynucleotide binding domain derived from a helicase. For instance, the polynucleotide binding moiety preferably comprises the sequence shown in SEQ ID NOs: 105 or 106 or a variant thereof. A variant of SEQ ID NOs: 105 or 106 is a protein that has an amino acid sequence which varies from that of SEQ ID NOs: 105 or 106 and which retains polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NOs: 105 or 106, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOs: 105 or 106 over the entire sequence. There may be at least 80%, for example at least 85%, 90% or 95%, amino acid identity over a stretch of 40 or more, for example 50, 60, 70 or 80 or more, contiguous amino acids (“hard homology”). Homology is determined as described below. The variant may differ from the wild-type sequence in any of the ways discussed below with reference to SEQ ID NOs: 2 and 4.

The topoisomerase is preferably a member of any of the Moiety Classification (EC) groups 5.99.1.2 and 5.99.1.3.

The polynucleotide binding moiety may be any of the enzymes discussed above.

The moiety may be labelled with a revealing label. The label may be any of those described above.

The moiety may be isolated from any moiety-producing organism, such as E. coli, T. thermophilus or bacteriophage, or made synthetically or by recombinant means. For example, the moiety may be synthesized by in vitro translation and transcription as described below. The moiety may be produced in large scale following purification as described below.

### Helicase Oligomers

As will be clear from the discussion above, the polynucleotide binding moiety is preferably derived from a helicase. For instance, it may be a polynucleotide domain from a helicase. The moiety more preferably comprises one or more helicases. The helicases may be any of those discussed above, including the helicases of the invention. In such embodiments, the constructs of the invention of course comprise two or more helicases attached together where at least one of the helicases is modified in accordance with the invention. The constructs may comprise two, three, four, five or more helicases. In other words, the constructs of the invention may comprise a helicase dimer, a helicase trimer, a helicase tetramer, a helicase pentamer and the like.

The two or more helicases can be attached together in any orientation. Identical or similar helicases may be attached via the same amino acid position or spatially proximate amino acid positions in each helicase. This is termed the “head-to-head” formation. Alternatively, identical or similar helicases may be attached via positions on opposite or different sides of each helicase. This is termed the “head-to-tail” formation. Helicase trimers comprising three identical or similar helicases may comprise both the head-to-head and head-to-tail formations.

The two or more helicases may be different from one another (i.e. the construct is a hetero-dimer, -trimer, -tetramer or -pentamer etc.). For instance, the constructs of the invention may comprise: (a) one or more Hel308 helicases and one or more XPD helicases; (b) one or more Hel308 helicases and one or more RecD helicases; (c) one or more Hel308 helicases and one or more TraI helicases; (d) one or more XPD helicases and one or more RecD helicases; (e) one or more XPD helicases and one or more TraI helicases; or (f) one or more RecD helicases and one or more TraI helicases. The construct may comprise two different variants of the same helicase. For instance, the construct may comprise two variants of one of the helicases discussed above with one or more cysteine residues or Faz residues introduced at different positions in each variant. In this instance, the helicases can be in a head-to-tail formation. In a preferred embodiment, a variant of SEQ ID NO: 10 comprising Q442C may be attached via cysteine linkage to a variant of SEQ ID NO: 10 comprising Q557C. Cys mutants of Hel308Mbu can also be made into hetero-dimers if necessary. In this approach, two different Cys mutant pairs such as Hel308Mbu-Q442C and Hel308Mbu-Q577C can be linked in head-to-tail fashion. Hetero-dimers can be formed in two possible ways. The first involves the use of a homo-bifunctional linker as discussed above. One of the helicase variants can be modified with a large excess of linker in such a way that one linker is attached to one molecule of the protein. This linker modified variant can then be purified away from unmodified proteins, possible homo-dimers and unreacted linkers to react with the other helicase variant. The resulting dimer can then be purified away from other species.

The second involves the use of hetero-bifunctional linkers. For example, one of the helicase variants can be modified with a first PEG linker containing maleimide or iodoacetamide functional group at one end and a cyclooctyne functional group (DIBO) at the other end. An example of this is shown below:

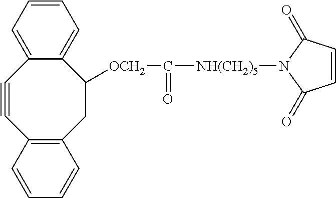

The second helicase variant can be modified with a second PEG linker containing maleimide or iodoacetamide functional group at one end and an azide functional group at the other end. An example is show below:

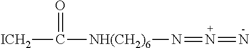

The two helicase variants with two different linkers can then be purified and clicked together (using copper free click chemistry) to make a dimer. Copper free click chemistry has been used in these applications because of its desirable properties. For example, it is fast, clean and not poisonous towards proteins. However, other suitable bio-orthogonal chemistries include, but are not limited to, Staudinger chemistry, hydrazine or hydrazide/aldehyde or ketone reagents (HyNic+4FB chemistry, including all Solulink™ reagents), Diels-Alder reagent pairs and boronic acid/salicyhydroxamate reagents.

These two ways of linking two different variants of the same helicase are also valid for any of the constructs discussed above in which the helicase and the moiety are different from one another, such as dimers of two different helicases and a helicase-polymerase dimer.

Similar methodology may also be used for linking different Faz variants. One Faz variant (such as SEQ ID NO: 10 comprising Q442Faz) can be modified with a large excess of linker in such a way that one linker is attached to one molecule of the protein. This linker modified Faz variant can then be purified away from unmodified proteins, possible homo-dimers and unreacted linkers to react with the second Faz variant (such as SEQ ID NO: 10 comprising Q577Faz). The resulting dimer can then be purified away from other species.

Hetero-dimers can also be made by linking cysteine variants and Faz variants of the same helicase or different helicases. For example, any of the above cysteine variants (such as SEQ ID NO: 10 comprising Q442C) can be used to make dimers with any of the above Faz variants (such SEQ ID NO: 10 comprising Q577Faz). Hetero-bifunctional PEG linkers with maleimide or iodoacetamide functionalities at one end and DBCO functionality at the other end can be used in this combination of mutants. An example of such a linker is shown below (DBCO-PEG4-maleimide):

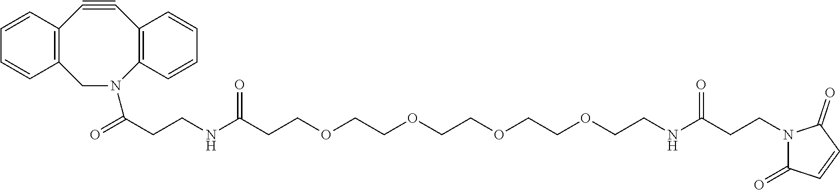 [SMILES: O=C(CCOCCOCCOCCOCCNC(=O)CCN1C(=O)C=CC1=O)NCCC(=O)N1Cc2ccccc2C#Cc2ccccc21]

The length of the linker can be varied by changing the number of PEG units between the two functional groups.

Helicase hetero-trimers can comprise three different types of helicases selected from Hel308 helicases, XPD helicases, RecD helicases, TraI helicases and variants thereof. The same is true for oligomers comprising more than three helicases. The two or more helicases within a construct may be different variants of the same helicase, such as different variants of SEQ ID NO: 10, 22, 33 or 52. The different variants may be modified at different positions to facilitate attachment via the different positions. The hetero-trimers may therefore be in a head-to-tail and head-to-head formation.

The two or more helicases in the constructs of the invention may be the same as one another (i.e. the construct is a homo-dimer, -trimer, -tetramer or -pentamer etc.) Homo-oligomers can comprise two or more Hel308 helicases, two or more XPD helicases, two or more RecD helicases, two or more TraI helicases or two or more of any of the variants discussed above. In such embodiments, the helicases are preferably attached using the same position in each helicase. The helicases are therefore attached head-to-head. The helicases may be linked using a cysteine residue or a Faz residue that has been substituted into the helicases at the same position. Cysteine residues in identical helicase variants can be linked using a homo-bifunctional linker containing thiol reactive groups such as maleimide or iodoacetamide. These functional groups can be at the end of a polyethyleneglycol (PEG) chain as in the following example:

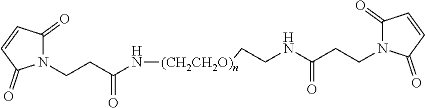

The length of the linker can be varied to suit the required applications. For example, n can be 2, 3, 4, 8, 11, 12, 16 or more. PEG linkers are suitable because they have favourable properties such as water solubility. Other non PEG linkers can also be used in cysteine linkage.

By using similar approaches, identical Faz variants can also be made into homo-dimers. Homo-bifunctional linkers with DIBO functional groups can be used to link two molecules of the same Faz variant to make homo-dimers using Cu²⁺ free click chemistry. An example of a linker is given below:

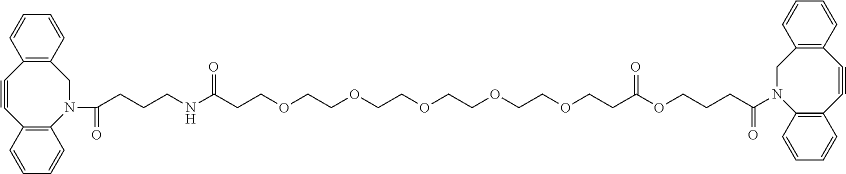 [SMILES: O=C(CCOCCOCCOCCOCCOCCC(=O)OCCCC(=O)N1Cc2ccccc2C#Cc2ccccc21)NCCCC(=O)N1Cc2ccccc2C#Cc2ccccc21]

The length of the PEG linker can vary to include 2, 4, 8, 12, 16 or more PEG units. Such linkers can also be made to incorporate a florescent tag to ease quantifications. Such fluorescence tags can also be incorporated into Maleimide linkers.

The invention also provides a construct comprising a helicase of the invention and an amino acid sequence comprising SEQ ID NO: 130 (H-L domains from Topoisomerase V from Methanopyrus kandleri; SEQ ID NO: 129) or a variant thereof having at least 80% homology to SEQ ID NO: 130 based on amino acid identity over the entire sequence of SEQ ID NO: 130, wherein the helicase is attached to the amino acid sequence and the construct has the ability to control the movement of a polynucleotide. The helicase may be attached to the amino acid sequence in any of the ways discussed above.

Preferred constructs of the invention are shown in the Table 6 below. Each row shows a preferred construct in which the helicase in the left-hand column is attached to additional polynucleotide binding moiety in the right-hand column in accordance with the invention. If the polynucleotide binding moiety in the right-hand column is a helicase, it may also be a helicase of the invention.

| | Additional polynucleotide |
|---|---|
| Helicase of the invention | binding moiety |
| Hel308 helicase of the invention as | Polymerase (preferably SEQ |
| defined above (preferably SEQ ID | ID NO: 101 or a variant |
| NO: 10, 22, 33 or 52 or a variant | thereof as defined above) |
| thereof as defined above) | |
| TraI helicase of the invention as | Polymerase (preferably SEQ |
| defined above (preferably SEQ ID | ID NO: 101 or a variant |
| NO: 85, 126, 134 and 138 or a | thereof as defined above) |
| variant thereof as defined above) | |
| Hel308 helicase of the invention as | Hel308 helicase as defined above |
| defined above (preferably SEQ ID | (preferably SEQ ID NO: 10, 22, |
| NO: 10, 22, 33 or 52 or a variant | 33 or 52 or a variant thereof |
| thereof as defined above) | as defined above) |
| TraI helicase of the invention as | TraI helicase as defined above |
| defined above (preferably SEQ ID | (preferably SEQ ID NO: 85, 126, |
| NO: 85, 126, 134 and 138 or a | 134 and 138 or a variant |
| variant thereof as defined above) | thereof as defined above) |
| Hel308 helicase of the invention as | TraI helicase as defined above |
| defined above (preferably SEQ ID | (preferably SEQ ID NO: 85, 126, |
| NO: 10, 22, 33 or 52 or a variant | 134 and 138 or a variant |
| thereof as defined above) | thereof as defined above) |
| TraI helicase of the invention as | Hel308 helicase as defined above |
| defined above (preferably SEQ ID | (preferably SEQ ID NO: 10, 22, |
| NO: 85, 126, 134 and 138 or a | 33 or 52 or a variant thereof |
| variant thereof as defined above) | as defined above) |

### Polynucleotide Sequences

Any of the proteins described herein may be expressed using methods known in the art. Polynucleotide sequences may be isolated and replicated using standard methods in the art. Chromosomal DNA may be extracted from a helicase producing organism, such as Methanococcoides burtonii, and/or a SSB producing organism, such as E. coli. The gene encoding the sequence of interest may be amplified using PCR involving specific primers. The amplified sequences may then be incorporated into a recombinant replicable vector such as a cloning vector. The vector may be used to replicate the polynucleotide in a compatible host cell. Thus polynucleotide sequences may be made by introducing a polynucleotide encoding the sequence of interest into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into a suitable expression vector. In an expression vector, the polynucleotide sequence is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

### Methods of the Invention

The invention provides a method of controlling the movement of a target polynucleotide. The method comprises contacting the target polynucleotide with a helicase of the invention or a construct of the invention and thereby controlling the movement of the polynucleotide. The method is preferably carried out with a potential applied across the pore. As discussed in more detail below, the applied potential typically results in the formation of a complex between the pore and the helicase or construct. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11; 129(27):8650-5.

The invention also provides a method of characterising a target polynucleotide. The method comprises (a) contacting the target polynucleotide with a transmembrane pore and a helicase of the invention or a construct of the invention such that the helicase or construct controls the movement of the target polynucleotide through the pore. The method also comprises (b) taking one or more measurements as the polynucleotide moves with respect to the pore wherein the measurements are indicative of one or more characteristics of the target polynucleotide and thereby characterising the target polynucleotide.

Steps (a) and (b) are preferably carried out with a potential applied across the pore as discussed above. In some instances, the current passing through the pore as the polynucleotide moves with respect to the pore is used to determine the sequence of the target polynucleotide. This is Strand Sequencing.

The method of the invention is for characterising a target polynucleotide. A polynucleotide is defined above.

The whole or only part of the target polynucleotide may be characterised using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, sea water or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well in the membrane along which or into which hydrated ions may flow.

Any transmembrane pore may be used in the invention. The pore may be biological or artificial. Suitable pores include, but are not limited to, protein pores, polynucleotide pores and solid state pores.

Any membrane may be used in accordance with the invention. Suitable membranes are well-known in the art. The membrane is preferably an amphiphilic layer. An amphiphilic layer is a layer formed from amphiphilic molecules, such as phospholipids, which have both at least one hydrophilic portion and at least one lipophilic or hydrophobic portion. The amphiphilic molecules may be synthetic or naturally occurring. Non-naturally occurring amphiphiles and amphiphiles which form a monolayer are known in the art and include, for example, 7s (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer.

The amphiphilic layer may be a monolayer or a bilayer. The amphiphilic layer is typically a planar lipid bilayer or a supported bilayer.

The amphiphilic layer is typically a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial lipid bilayer. The layer may comprise other transmembrane and/or intramembrane proteins as well as other molecules in addition to the pore. Suitable apparatus and conditions are discussed below. The method of the invention is typically carried out in vitro.

The polynucleotide may be coupled to the membrane. This may be done using any known method. If the membrane is an amphiphilic layer, such as a lipid bilayer (as discussed in detail above), the polynucleotide is preferably coupled to the membrane via a polypeptide present in the membrane or a hydrophobic anchor present in the membrane. The hydrophobic anchor is preferably a lipid, fatty acid, sterol, carbon nanotube or amino acid.

The polynucleotide may be coupled directly to the membrane. The polynucleotide is preferably coupled to the membrane via a linker. Preferred linkers include, but are not limited to, polymers, such as polynucleotides, polyethylene glycols (PEGs) and polypeptides. If a polynucleotide is coupled directly to the membrane, then some data will be lost as the characterising run cannot continue to the end of the polynucleotide due to the distance between the membrane and the helicase. If a linker is used, then the polynucleotide can be processed to completion. If a linker is used, the linker may be attached to the polynucleotide at any position. The linker is typically attached to the polynucleotide at the tail polymer.

The coupling may be stable or transient. For certain applications, the transient nature of the coupling is preferred. If a stable coupling molecule were attached directly to either the 5′ or 3′ end of a polynucleotide, then some data will be lost as the characterising run cannot continue to the end of the polynucleotide due to the distance between the bilayer and the helicase's active site. If the coupling is transient, then when the coupled end randomly becomes free of the bilayer, then the polynucleotide can be processed to completion. Chemical groups that form stable or transient links with the membrane are discussed in more detail below. The polynucleotide may be transiently coupled to an amphiphilic layer, such as a lipid bilayer using cholesterol or a fatty acyl chain. Any fatty acyl chain having a length of from 6 to 30 carbon atoms, such as hexadecanoic acid, may be used.

In preferred embodiments, the polynucleotide is coupled to an amphiphilic layer. Coupling of polynucleotides to synthetic lipid bilayers has been carried out previously with various different tethering strategies. These are summarised in Table 7 below.

TABLE 7

| Attachment | Type of | |
|---|---|---|
| group | coupling | Reference |
| Thiol | Stable | Yoshina-Ishii, C. and S. G. Boxer (2003). |
| | | “Arrays of mobile tethered vesicles |
| | | on supported lipid bilayers.” |
| | | J Am Chem Soc 125(13): 3696-7. |
| Biotin | Stable | Nikolov, V., R. Lipowsky, et al. (2007). |
| | | “Behavior of giant vesicles with |
| | | anchored DNA molecules.” |
| | | Biophys J 92(12): 4356-68 |
| Cholesterol | Transient | Pfeiffer, I. and F. Hook (2004). |
| | | “Bivalent cholesterol-based |
| | | coupling of oligonucletides to |
| | | lipid membrane assemblies.” |
| | | J Am Chem Soc 126(33): 10224-5 |
| Lipid | Stable | van Lengerich, B., R. J. Rawle, et al. |
| | | “Covalent attachment of lipid |
| | | vesicles to a fluid-supported bilayer |
| | | allows observation of DNA-mediated |
| | | vesicle interactions.” |
| | | Langmuir 26(11): 8666-72 |

Polynucleotides may be functionalized using a modified phosphoramidite in the synthesis reaction, which is easily compatible for the addition of reactive groups, such as thiol, cholesterol, lipid and biotin groups. These different attachment chemistries give a suite of attachment options for polynucleotides. Each different modification group tethers the polynucleotide in a slightly different way and coupling is not always permanent so giving different dwell times for the polynucleotide to the bilayer. The advantages of transient coupling are discussed above.

Coupling of polynucleotides can also be achieved by a number of other means provided that a reactive group can be added to the polynucleotide. The addition of reactive groups to either end of DNA has been reported previously. A thiol group can be added to the 5′ of ssDNA using polynucleotide kinase and ATPTS (Grant, G. P. and P. Z. Qin (2007). “A facile method for attaching nitroxide spin labels at the 5′ terminus of nucleic acids.” Nucleic Acids Res 35(10): e77). A more diverse selection of chemical groups, such as biotin, thiols and fluorophores, can be added using terminal transferase to incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169(2): 376-82).

Alternatively, the reactive group could be considered to be the addition of a short piece of DNA complementary to one already coupled to the bilayer, so that attachment can be achieved via hybridisation. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89(20): 9823-5). Alternatively either ssDNA or dsDNA could be ligated to native dsDNA and then the two strands separated by thermal or chemical denaturation. To native dsDNA, it is possible to add either a piece of ssDNA to one or both of the ends of the duplex, or dsDNA to one or both ends. Then, when the duplex is melted, each single strand will have either a 5′ or 3′ modification if ssDNA was used for ligation or a modification at the 5′ end, the 3′ end or both if dsDNA was used for ligation. If the polynucleotide is a synthetic strand, the coupling chemistry can be incorporated during the chemical synthesis of the polynucleotide. For instance, the polynucleotide can be synthesized using a primer with a reactive group attached to it.

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. By using an antisense primer that has a reactive group, such as a cholesterol, thiol, biotin or lipid, each copy of the amplified target DNA will contain a reactive group for coupling.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as 6, 7, 8 or 9 subunits. The pore is preferably a hexameric, heptameric, octameric or nonameric pore.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from Msp or from α-hemolysin (α-HL).

The transmembrane protein pore is preferably derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp typically comprises the sequence shown in SEQ ID NO: 2 or a variant thereof. SEQ ID NO: 2 is the MS-(B1)8 mutant of the MspA monomer. It includes the following mutations: D90N, D91N, D93N, D118R, D134R and E139K. A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (ncbi.nlm.nih.gov/).

SEQ ID NO: 2 is the MS-(B1)8 mutant of the MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB: A138P. The variant may comprise one or more of the following substitutions present in MspC: A96G, N102E and A138P. The variant may comprise one or more of the following mutations present in MspD: Deletion of G1, L2V, E5Q, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D. The variant preferably comprises the mutation L88N. A variant of SEQ ID NO: 2 has the mutation L88N in addition to all the mutations of MS-(B1)8 and is called MS-(B2)8. The pore used in the invention is preferably MS-(B2)8. The further preferred variant comprises the mutations G75S/G77S/L88N/Q126R. The variant of SEQ ID NO: 2 has the mutations G75S/G77S/L88N/Q126R in addition to all the mutations of MS-(B1)8 and is called MS-(B2C)8. The pore used in the invention is preferably MS-(B2)8 or MS-(B2C)8.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 8 below. Where amino acids have similar polarity, this can also be determined by reference to the hydropathy scale for amino acid side chains in Table 9.

TABLE 8
Chemical properties of amino acids

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 9
Hydropathy scale

| Side | |
|---|---|
| Chain | Hydropathy |
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues 88, 90, 91, 105, 118 and 134 of SEQ ID NO: 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which retains its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant retains its ability to form a pore. A variant of SEQ ID NO: 2 preferably includes one or more modifications, such as substitutions, additions or deletions, within its α-helices and/or loop regions.

The monomers derived from Msp may be modified to assist their identification or purification, for example by the addition of histidine residues (a hist tag), aspartic acid residues (an asp tag), a streptavidin tag or a flag tag, or by the addition of a signal sequence to promote their secretion from a cell where the polypeptide does not naturally contain such a sequence. An alternative to introducing a genetic tag is to chemically react a tag onto a native or engineered position on the pore. An example of this would be to react a gel-shift reagent to a cysteine engineered on the outside of the pore. This has been demonstrated as a method for separating hemolysin hetero-oligomers (Chem Biol. 1997 July; 4(7):497-505).

The monomer derived from Msp may be labelled with a revealing label. The revealing label may be any suitable label which allows the pore to be detected. Suitable labels are described above.

The monomer derived from Msp may also be produced using D-amino acids. For instance, the monomer derived from Msp may comprise a mixture of L-amino acids and D-amino acids. This is conventional in the art for producing such proteins or peptides.

The monomer derived from Msp contains one or more specific modifications to facilitate nucleotide discrimination. The monomer derived from Msp may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomer derived from Msp can be produced using standard methods known in the art. The monomer derived from Msp may be made synthetically or by recombinant means. For example, the pore may be synthesized by in vitro translation and transcription (IVTT). Suitable methods for producing pores are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

The transmembrane protein pore is also preferably derived from α-hemolysin (α-HL). The wild type α-HL pore is formed of seven identical monomers or subunits (i.e. it is heptameric). The sequence of one monomer or subunit of α-hemolysin-NN is shown in SEQ ID NO: 4. The transmembrane protein pore preferably comprises seven monomers each comprising the sequence shown in SEQ ID NO: 4 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 4 form loop regions. Residues 113 and 147 of SEQ ID NO: 4 form part of a constriction of the barrel or channel of (α-HL.

In such embodiments, a pore comprising seven proteins or monomers each comprising the sequence shown in SEQ ID NO: 4 or a variant thereof are preferably used in the method of the invention. The seven proteins may be the same (homo-heptamer) or different (hetero-heptamer).

A variant of SEQ ID NO: 4 is a protein that has an amino acid sequence which varies from that of SEQ ID NO: 4 and which retains its pore forming ability. The ability of a variant to form a pore can be assayed using any method known in the art. For instance, the variant may be inserted into an amphiphilic layer, such as a lipid bilayer, along with other appropriate subunits and its ability to oligomerise to form a pore may be determined. Methods are known in the art for inserting subunits into amphiphilic layers, such as lipid bilayers. Suitable methods are discussed above.

The variant may include modifications that facilitate covalent attachment to or interaction with the helicase or construct. The variant preferably comprises one or more reactive cysteine residues that facilitate attachment to the helicase or construct. For instance, the variant may include a cysteine at one or more of positions 8, 9, 17, 18, 19, 44, 45, 50, 51, 237, 239 and 287 and/or on the amino or carboxy terminus of SEQ ID NO: 4. Preferred variants comprise a substitution of the residue at position 8, 9, 17, 237, 239 and 287 of SEQ ID NO: 4 with cysteine (A8C, T9C, N17C, K237C, S239C or E287C). The variant is preferably any one of the variants described in International Application No. PCT/GB09/001690 (published as WO 2010/004273), PCT/GB09/001679 (published as WO 2010/004265) or PCT/GB10/000133 (published as WO 2010/086603).

The variant may also include modifications that facilitate any interaction with nucleotides.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 4, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 4 over the entire sequence. There may be at least 80%, for example at least 85%, 90% or 95%, amino acid identity over a stretch of 200 or more, for example 230, 250, 270 or 280 or more, contiguous amino acids (“hard homology”). Homology can be determined as discussed above.

Amino acid substitutions may be made to the amino acid sequence of SEQ ID NO: 4 in addition to those discussed above, for example up to 1, 2, 3, 4, 5, 10, 20 or 30 substitutions. Conservative substitutions may be made as discussed above.

One or more amino acid residues of the amino acid sequence of SEQ ID NO: 4 may additionally be deleted from the polypeptides described above. Up to 1, 2, 3, 4, 5, 10, 20 or 30 residues may be deleted, or more.

Variants may be fragments of SEQ ID NO: 4. Such fragments retain pore-forming activity. Fragments may be at least 50, 100, 200 or 250 amino acids in length. A fragment preferably comprises the pore-forming domain of SEQ ID NO: 4. Fragments typically include residues 119, 121, 135. 113 and 139 of SEQ ID NO: 4.

One or more amino acids may be alternatively or additionally added to the polypeptides described above. An extension may be provided at the amino terminus or carboxy terminus of the amino acid sequence of SEQ ID NO: 4 or a variant or fragment thereof. The extension may be quite short, for example from 1 to 10 amino acids in length. Alternatively, the extension may be longer, for example up to 50 or 100 amino acids. A carrier protein may be fused to a pore or variant.

As discussed above, a variant of SEQ ID NO: 4 is a subunit that has an amino acid sequence which varies from that of SEQ ID NO: 4 and which retains its ability to form a pore. A variant typically contains the regions of SEQ ID NO: 4 that are responsible for pore formation. The pore forming ability of α-HL, which contains a β-barrel, is provided by β-strands in each subunit. A variant of SEQ ID NO: 4 typically comprises the regions in SEQ ID NO: 4 that form β-strands. The amino acids of SEQ ID NO: 4 that form β-strands are discussed above. One or more modifications can be made to the regions of SEQ ID NO: 4 that form β-strands as long as the resulting variant retains its ability to form a pore. Specific modifications that can be made to the β-strand regions of SEQ ID NO: 4 are discussed above.

A variant of SEQ ID NO: 4 preferably includes one or more modifications, such as substitutions, additions or deletions, within its α-helices and/or loop regions. Amino acids that form α-helices and loops are discussed above.

The variant may be modified to assist its identification or purification as discussed above.

Pores derived from α-HL can be made as discussed above with reference to pores derived from Msp.

In some embodiments, the transmembrane protein pore is chemically modified. The pore can be chemically modified in any way and at any site. The transmembrane protein pore is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein pore may be chemically modified by the attachment of any molecule. For instance, the pore may be chemically modified by attachment of a dye or a fluorophore.

Any number of the monomers in the pore may be chemically modified. One or more, such as 2, 3, 4, 5, 6, 7, 8, 9 or 10, of the monomers is preferably chemically modified as discussed above.

The reactivity of cysteine residues may be enhanced by modification of the adjacent residues. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the pore before a linker is attached.

The molecule (with which the pore is chemically modified) may be attached directly to the pore or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

The helicase or construct may be covalently attached to the pore. The helicase or construct is preferably not covalently attached to the pore. The application of a voltage to the pore and helicase or construct typically results in the formation of a sensor that is capable of sequencing target polynucleotides. This is discussed in more detail below.

Any of the proteins described herein, i.e. the helicases, the transmembrane protein pores or constructs, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the helicase, pore or construct. An example of this would be to react a gel-shift reagent to a cysteine engineered on the outside of the pore. This has been demonstrated as a method for separating hemolysin hetero-oligomers (Chem Biol. 1997 July; 4(7):497-505).

The helicase, pore or construct may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Proteins may be made synthetically or by recombinant means. For example, the helicase, pore or construct may be synthesized by in vitro translation and transcription (IVTT). The amino acid sequence of the helicase, pore or construct may be modified to include non-naturally occurring amino acids or to increase the stability of the protein. When a protein is produced by synthetic means, such amino acids may be introduced during production. The helicase, pore or construct may also be altered following either synthetic or recombinant production.

The helicase, pore or construct may also be produced using D-amino acids. For instance, the pore or construct may comprise a mixture of L-amino acids and D-amino acids. This is conventional in the art for producing such proteins or peptides.

The helicase, pore or construct may also contain other non-specific modifications as long as they do not interfere with pore formation or helicase or construct function. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

The helicase, pore and construct can be produced using standard methods known in the art. Polynucleotide sequences encoding a helicase, pore or construct may be derived and replicated using standard methods in the art. Polynucleotide sequences encoding a helicase, pore or construct may be expressed in a bacterial host cell using standard techniques in the art. The helicase, pore and/or construct may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The helicase, pore and/or construct may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

The method of the invention involves measuring one or more characteristics of the target polynucleotide. The method may involve measuring two, three, four or five or more characteristics of the target polynucleotide. The one or more characteristics are preferably selected from (i) the length of the target polynucleotide, (ii) the identity of the target polynucleotide, (iii) the sequence of the target polynucleotide, (iv) the secondary structure of the target polynucleotide and (v) whether or not the target polynucleotide is modified. Any combination of (i) to (v) may be measured in accordance with the invention.

For (i), the length of the polynucleotide may be measured for example by determining the number of interactions between the target polynucleotide and the pore or the duration of interaction between the target polynucleotide and the pore.

For (ii), the identity of the polynucleotide may be measured in a number of ways. The identity of the polynucleotide may be measured in conjunction with measurement of the sequence of the target polynucleotide or without measurement of the sequence of the target polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the target polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106(19):7702-7, Lieberman K R et al, J Am Chem Soc. 2010; 132(50):17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the target polynucleotide is modified by methylation, by oxidation, by damage, with one or more proteins or with one or more labels, tags or spacers. Specific modifications will result in specific interactions with the pore which can be measured using the methods described below. For instance, methylcytosine may be distinguished from cytosine on the basis of the current flowing through the pore during its interaction with each nucleotide.

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and International Application WO-2000/28312. Alternatively, electrical measurements may be made using a multi-channel system, for example as described in International Application WO-2009/077734 and International Application WO-2011/067559.

In a preferred embodiment, the method comprises:
(a) contacting the target polynucleotide with a transmembrane pore and a helicase of the invention or a construct of the invention such that the target polynucleotide moves through the pore and the helicase or construct controls the movement of the target polynucleotide through the pore; and
(b) measuring the current passing through the pore as the polynucleotide moves with respect to the pore wherein the current is indicative of one or more characteristics of the target polynucleotide and thereby characterising the target polynucleotide.

The methods may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The methods may be carried out using the apparatus described in International Application No. PCT/GB08/000562 (WO 2008/102120).

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. Hel308, XPD, RecD and TraI helicases surprisingly work under high salt concentrations. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

The method may be carried out in the presence of free nucleotides or free nucleotide analogues and/or an enzyme cofactor that facilitates the action of the helicase or construct. The method may also be carried out in the absence of free nucleotides or free nucleotide analogues and in the absence of an enzyme cofactor. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the helicase or construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The target polynucleotide may be contacted with the helicase or construct and the pore in any order. In is preferred that, when the target polynucleotide is contacted with the helicase or construct and the pore, the target polynucleotide firstly forms a complex with the helicase or construct. When the voltage is applied across the pore, the target polynucleotide/helicase or construct complex then forms a complex with the pore and controls the movement of the polynucleotide through the pore.

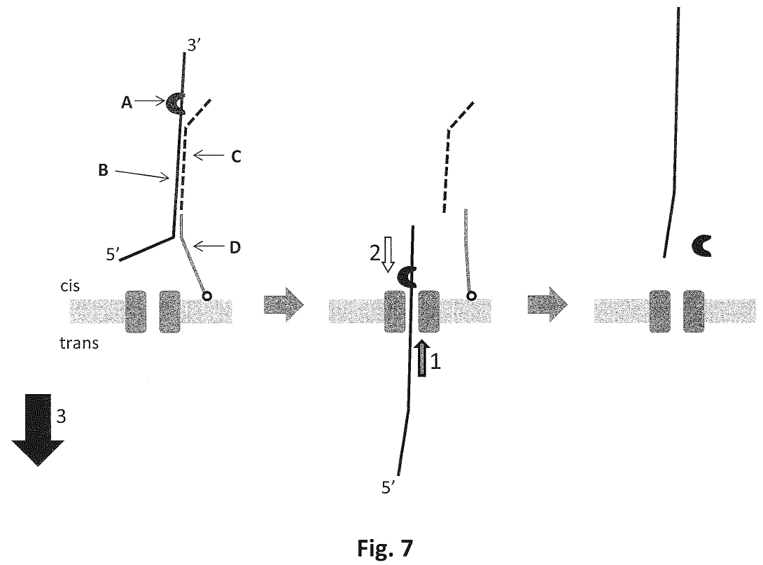

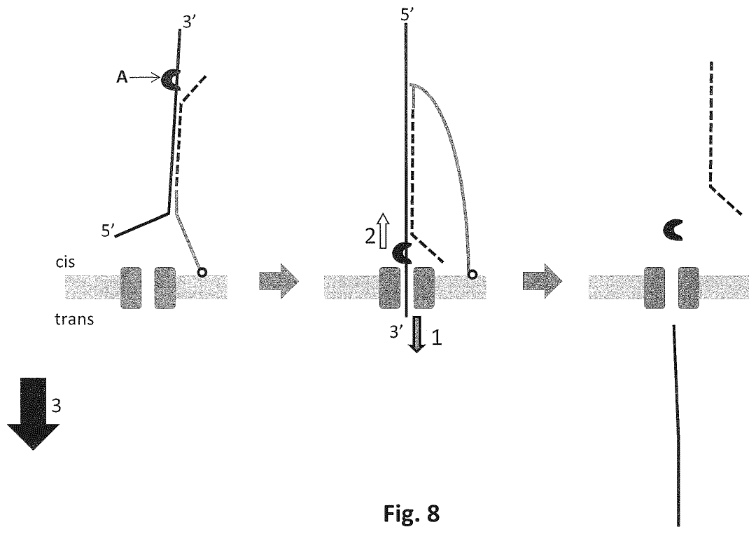

As discussed above, helicases may work in two modes with respect to the pore. The helicases of the invention or the constructs of the invention can also work in two modes. First, the method is preferably carried out using the helicase or construct such that it moves the target sequence through the pore with the field resulting from the applied voltage. In this mode the 3′ end of the DNA is first captured in the pore (for a 3′-5′ helicase), and the helicase or construct moves the DNA into the pore such that the target sequence is passed through the pore with the field until it finally translocates through to the trans side of the bilayer (See FIG. 8). Alternatively, the method is preferably carried out such that the helicase or construct moves the target sequence through the pore against the field resulting from the applied voltage. In this mode the 5′ end of the DNA is first captured in the pore (for a 3′-5′ helicase), and the helicase or construct moves the DNA through the pore such that the target sequence is pulled out of the pore against the applied field until finally ejected back to the cis side of the bilayer (see FIG. 7).

### Other Methods

The invention also provides a method of forming a sensor for characterising a target polynucleotide. The method comprises forming a complex between a pore and a helicase of the invention or a construct of the invention. The complex may be formed by contacting the pore and the helicase or construct in the presence of the target polynucleotide and then applying a potential across the pore. The applied potential may be a chemical potential or a voltage potential as described above. Alternatively, the complex may be formed by covalently attaching the pore to the helicase or construct. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target polynucleotide. The method preferably comprises forming a complex between a pore derived from Msp and a helicase of the invention or a construct of the invention. Any of the embodiments discussed above with reference to the methods of the invention equally apply to this method. The invention also provides a sensor produced using the method of the invention.

### Kits

The present invention also provides a kit for characterising a target polynucleotide. The kit comprises (a) a pore and (b) a helicase of the invention or a construct of the invention. Any of the embodiments discussed above with reference to the method of the invention equally apply to the kits.

The kit may further comprise the components of a membrane, such as the phospholipids needed to form an amphiphilic layer, such as a lipid bilayer.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

### Apparatus

The invention also provides an apparatus for characterising a target polynucleotide. The apparatus comprises a plurality of pores and a plurality of helicases of the invention or a plurality of constructs of the invention. The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention.

The apparatus is preferably set up to carry out the method of the invention.

The apparatus preferably comprises:
a sensor device that is capable of supporting the plurality of pores and being operable to perform polynucleotide characterisation using the pores and constructs; and
at least one reservoir for holding material for performing the characterisation.

The apparatus preferably comprises:
a sensor device that is capable of supporting the plurality of pores and being operable to perform polynucleotide characterisation using the pores and helicases or constructs; and
at least one reservoir for holding material for performing the characterisation.

The apparatus preferably comprises:
a sensor device that is capable of supporting the membrane and plurality of pores and being operable to perform polynucleotide characterising using the pores and helicases or constructs;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device. The apparatus may be any of those described in International Application No. No. PCT/GB08/004127 (published as WO 2009/077734), PCT/GB10/000789 (published as WO 2010/122293), International Application No. PCT/GB10/002206 (not yet published) or International Application No. PCT/US99/25679 (published as WO 00/28312). Methods of Producing Helicases of the Invention

The invention also provides methods of producing a helicase of the invention. In one embodiment, the method comprises providing a helicase formed from one or more monomers and comprising a polynucleotide binding domain which comprises an opening through which a polynucleotide can unbind from the helicase. Any of the helicases discussed above can be used in the methods.

The method also comprises modifying the helicase such that two or more parts on the same monomer of the helicase are connected to reduce the size of the opening. The site of and method of connection are selected as discussed above.

In another embodiment, the method comprises providing a Hel308 helicase. Any of the Hel308 helicases described above may be used.

The method further comprises introducing one or more cysteine residues and/or one or more non-natural amino acids at one or more of the positions which correspond to D272, N273, D274, G281, E284, E285, E287, S288, T289, G290, E291, D293, T294, N300, R303, K304, N314, 5315, N316, H317, R318, K319, L320, E322, R326, N328 and S615 in Hel308 Mbu (SEQ ID NO: 10).

The method preferably further comprises (c) heating the modified helicase, for instance by heating at 50° C. for 10 minutes, (d) exposing the modified helicase to UV light, for instance by exposing the modified helicase to high intensity UV light at 254 nm for about 10 to about 15 minutes or (e) exposing the modified helicase to ferrocyanide and ferricyanide, such as potassium ferrocyanide and potassium ferricyanide. Any combination of steps (c), (d) and (e) may be performed, such as (c), (d), (e), (c) and (d), (d) and (e), (c) and (e) or (c), (d) and (e).

The method preferably further comprises determining whether or not the helicase is capable of controlling the movement of a polynucleotide. Assays for doing this are described above. If the movement of a polynucleotide can be controlled, the helicase has been modified correctly and a helicase of the invention has been produced. If the movement of a polynucleotide cannot be controlled, a helicase of the invention has not been produced.

### Methods of Producing Constructs of the Invention

The invention also provides a method of producing a construct of the invention. The method comprises attaching, preferably covalently attaching a helicase of the invention to an additional polynucleotide binding moiety. Any of the helicases and moieties discussed above can be used in the methods. The site of and method of covalent attachment are selected as discussed above.

The method preferably further comprises determining whether or not the construct is capable of controlling the movement of a polynucleotide. Assays for doing this are described above. If the movement of a polynucleotide can be controlled, the helicase and moiety have been attached correctly and a construct of the invention has been produced. If the movement of a polynucleotide cannot be controlled, a construct of the invention has not been produced.

The following Example illustrates the invention.

### Example 1

This Example describes the method of synthesising the Hel308 Mbu (E284C/S615C)-bismaleimidePEG3 (SEQ ID NO: 10 with the mutations E284C/S615C connected by a bismaleimidePEG3 linker). In this case a covalent link between cysteines at positions 284 and 615 in the primary sequence of Hel308 Mbu (SEQ ID NO: 10) was made by reacting these positions with a bismaleimidePEG3 linker (approximately 3.7 nm in length).

In detail, 6 μl of 1 M DTT was added to 600 μL of Hel308 Mbu(E284C/S615C) (SEQ ID NO: 10 with the mutations E284C/S615C, stored in 50 mM Tris-HCl pH 8.0, 421 mM NaCl, 10% Glycerol, 10 mM DTT) and the mixture was incubated at room temperature on a 10″ wheel rotating at 20 rpm for 30 minutes. This mixture was buffer exchanged through Pierce 2 mL Zeba desalting columns, 7k MWCO into 100 mM potassium phosphate, 500 mM NaCl, 5 mM EDTA, 0.1% Tween-20 pH 8.0 to give 550 μL of sample. To this was added, 5.5 μL of bismaleimidePEG3 (QuantaBiodesign, Product Ref=10215) and the mixture incubated at room temperature on a 10″ wheel rotating at 20 rpm for 120 minutes. To stop the reaction, 5.5 μL of 1 M DTT was added to quench any remaining maleimides. Analysis of the reaction was by 7.5% polyacrylamide gel or by reverse phase HPLC (chromatographed on a Jupiter C5 300A 5 μm 150×4.6 mm column, using a gradient of acetonitrile in 0.1% TFA). FIG. 1 shows a coomassie stained 7.5% Tris-HCl gel (loaded with Laemmli loading buffer) of the Hel308 Mbu(E284C/S615C)-bismaleimidePEG3 (SEQ ID NO: 10 with the mutations E284C/S615C connected by a bismaleimidePEG3 linker) reaction mixture. Lane X shows an appropriate protein ladder (the mass unit markers are shown on the left of the gel). Lanes a-c contain 2 μL, 5 μL or 10 μL of approximately 2.5 μM Hel308 Mbu(E284C/S615C) monomer (SEQ ID NO: 10 with mutations E284C/S615C). Lanes d-f contain 2 μL, 5 μL or 10 μL of approximately 2.5 μM Hel308 Mbu(E284C/S615C)-bismaleimidePEG3 (SEQ ID NO: 10 with the mutations E284C/S615C connected by a bismaleimidePEG3 linker), it was clear from the gel that the reaction to attach the bismaleimidePEG3 linker went to nearly 100% yield. The Hel308 Mbu(E284C/S615C)-bismaleimidePEG3 (SEQ ID NO: 10 with the mutations E284C/S615C connected by a bismaleimidePEG3 linker) was then buffer exchanged to 50 mM Tris, 500 mM NaCl, 2 mM DTT, 10% glycerol pH 8.0.

### Example 2

This example describes the method of synthesising the Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide, SEQ ID NO: 109 corresponds to the peptide sequence SRDFWRS)). In this case a covalent link between cysteines at positions 284 and 615 in the primary sequence of Hel308 Mbu (SEQ ID NO: 10) was made by reacting these positions with a bismaleimide peptide linker (maleimide-propyl-SRDFWRS-(1,2-diaminoethane)-propyl-maleimide, SEQ ID NO: 109 corresponds to the peptide sequence SRDFWRS).

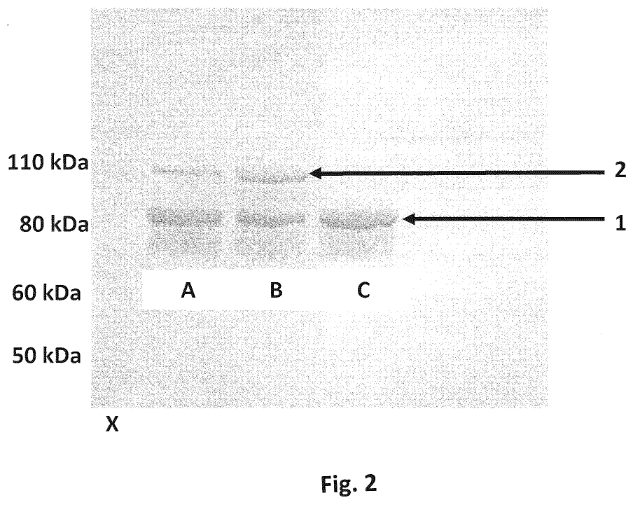

In detail, 2 μl of 1 M DTT was added to 200 μL of Hel308 Mbu(E284C/S615C) (SEQ ID NO: 10 with the mutations E284C/S615C, stored in 50 mM Tris-HCl pH 8.0, 421 mM NaCl, 10% Glycerol, 10 mM DTT) and the mixture was incubated at room temperature on a 10″ wheel rotating at 20 rpm for 30 minutes. This mixture was buffer exchanged through Pierce 2 mL Zeba desalting columns, 7k MWCO into 100 mM potassium phosphate, 500 mM NaCl, 5 mM EDTA, 0.1% Tween-20 pH 8.0 to give 540 μL of sample. To an aliquot of 100 ul, 0.5 ul of 10 mM maleimide-propyl-SRDFWRS-(1,2-diaminoethane)-propyl-maleimide (PPRL, Product Ref=16450) was added and the mixture incubated at room temperature on a 10″ wheel rotating at 20 rpm for 120 minutes. To stop the reaction, 1 ul of 1 M DTT was added to quench any remaining maleimides. Analysis of the reaction is by 7.5% polyacrylamide gel or by reverse phase HPLC (chromatographed on a Jupiter C5 300A 5 μm 150×4.6 mm column, using a gradient of acetonitrile in 0.1% TFA). FIG. 2 shows a coomassie stained 7.5% Tris-HCl gel of the Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide) reaction mixture. Lane X shows an appropriate protein ladder (the mass unit markers are shown on the left of the gel). Lane A contains 5 μL of approximately 10 μM Hel308 Mbu(E284C/S615C)-bismaleimidePEG3 (SEQ ID NO: 10 with the mutations E284C/S615C connected by a bismaleimidePEG3 linker) as a reference. The upper band corresponds to Hel308 Mbu(E284C/S615C)-bismaleimidePEG3 and the lower band to Hel308 Mbu (E284C/S615C) (SEQ ID NO: 10 with the mutations E284C/S615C). Lane B contains 5 μL of approximately 10 μM Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide), it was clear from the gel that the reaction to attach the mal-pep-mal linker did not go to completion as a band for the Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide) (upper band) and the Hel308 Mbu (E284C/S615C) (SEQ ID NO: 10 with the mutations E284C/S615C) (lower band) are observed. Lane C contains Hel308 Mbu (E284C/S615C) (SEQ ID NO: 10 with the mutations E284C/S615C).

The Hel308 Mbu (E284C/S615C)-mal-pep-mal (SEQ ID NO: 10 with the mutations E284C/S615C connected by a bismaleimide peptide linker (maleimide-propyl-SRDFWRS-(1,2-diaminoethane)-propyl-maleimide, SEQ ID NO: 109 corresponds to the peptide sequence SRDFWRS)) was then buffer exchanged to 50 mM Tris, 500 mM NaCl, 2 mM DTT, 10% glycerol pH 8.0.

### Example 3

This example compares the enzyme processivity of two Hel308 Mbu helicases in which the opening has been closed (Hel308 Mbu(E284C/S615C)-bismaleimidePEG11 (SEQ ID NO: 10 with mutations E284C/S615C connected by a bismaleimidePEG11) and Hel308 Mbu(E284C/S615C)-bismaleimidePEG3) (SEQ ID NO: 10 with the mutations E284C/S615C connected by a bismaleimidePEG3 linker) to that of the Hel308 Mbu monomer (SEQ ID NO: 10) using a fluorescence based assay.

### Materials and Methods

SEQ ID NOs: 110 to 114. SEQ ID NO: 112 has a carboxyfluorescein at the 5′ end and a black-hole quencher at the 3′ end.

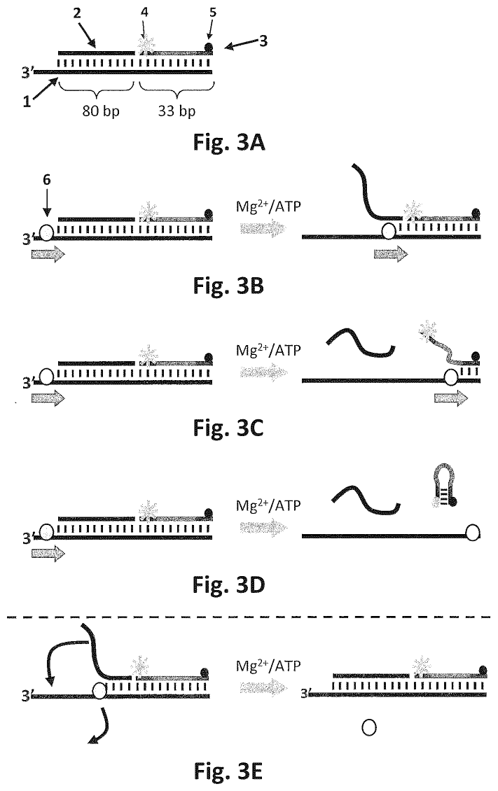

A custom fluorescent substrate was used to assay the ability of the helicase to displace hybridised dsDNA (FIGS. 3A-3E). The fluorescent substrate (50 nM final) has a 3′ ssDNA overhang, and 80 and 33 base-pair sections of hybridised dsDNA (FIG. 3A, SEQ ID NO: 110). The major lower “template” strand is hybridised to an 80 nt “blocker” strand (SEQ ID NO: 111), adjacent to its 3′ overhang, and a 33 nt fluorescent probe, labelled at its 5′ and 3′ ends with carboxyfluorescein (FAM) and black-hole quencher (BHQ-1) bases, respectively (SEQ ID NO: 112). When hybridised, the FAM is distant from the BHQ-1 and the substrate is essentially fluorescent. In the presence of ATP (1 mM) and MgCl₂ (10 mM), the helicase (10 nM) binds to the substrate's 3′ overhang (SEQ ID NO: 110), moves along the lower strand, and begins to displace the 80 nt blocker strand (SEQ ID NO: 111), as shown in FIG. 3B. If processive, the helicase displaces the fluorescent probe (SEQ ID NO: 112, labelled with a carboxyfluorescein (FAM) at its 5′ end a black-hole quencher (BHQ-1) at its 3′ end) too (FIG. 3C). The fluorescent probe is designed in such a way that its 5′ and 3′ ends are self-complementary and thus form a kinetically-stable hairpin once displaced, preventing the probe from re-annealing to the template strand (FIG. 3D). Upon formation of the hairpin product, the FAM is brought into the vicinity of the BHQ-1 and its fluorescence is quenched. A processive enzyme, capable of displacing the 80 mer “blocker” (SEQ ID NO: 111) and fluorescent (SEQ ID NO: 112, labelled with a carboxyfluorescein (FAM) at its 5′ end a black-hole quencher (BHQ-1) at its 3′ end) strands will therefore lead to a decrease in fluorescence over time. However, if the enzyme has a processivity of less than 80 nt it would be unable to displace the fluorescent strand (SEQ ID NO: 112, labelled with a carboxyfluorescein (FAM) at its 5′ end a black-hole quencher (BHQ-1) at its 3′ end) and, therefore, the “blocker” strand (SEQ ID NO: 111) would reanneal to the major bottom strand (FIG. 3E, SEQ ID NO: 110).

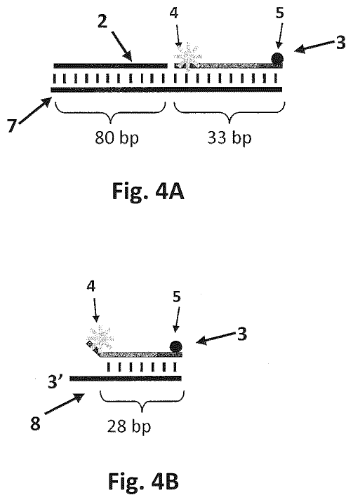

Additional custom fluorescent substrates were also used for control purposes. The substrate used as a negative control was identical to that of the one described in FIGS. 3A-3E but lacking the 3′overhang (FIG. 4A, (SEQ ID NOs: 111, 112 (labelled with a carboxyfluorescein (FAM) at its 5′ end a black-hole quencher (BHQ-1) at its 3′ end) and 113)). A similar substrate to that described in FIGS. 3A-3E but lacking the 80 base pair section, used as a positive control for active, but not necessarily processive, helicases (FIG. 4B, (SEQ ID NO's: 112 (labelled with a carboxyfluorescein (FAM) at its 5′ end a black-hole quencher (BHQ-1) at its 3′ end) and 114)).

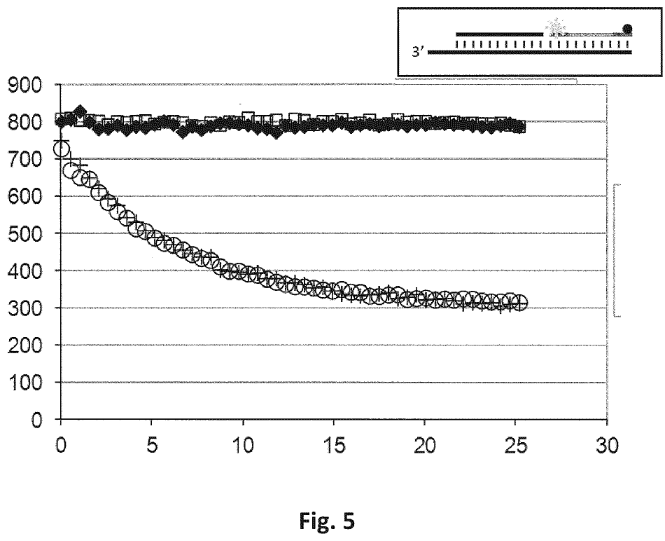

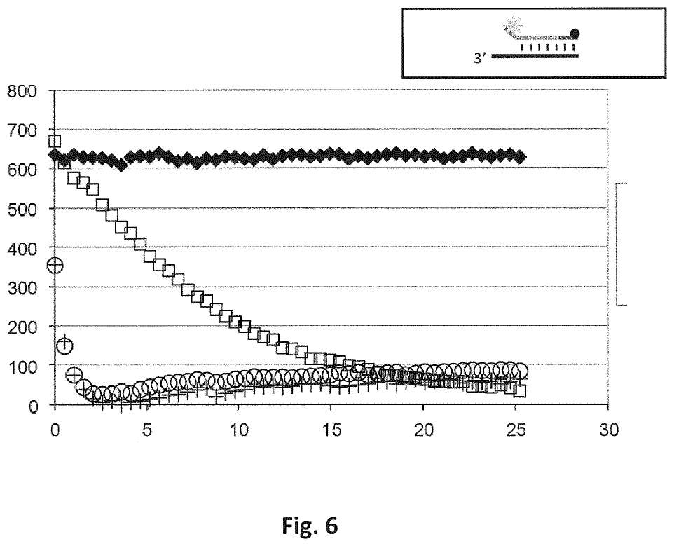

FIG. 5 shows a graph of the time-dependent fluorescence changes upon testing Hel308 Mbu monomer (SEQ ID NO: 10), Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker) against the processivity substrate shown in FIGS. 3A-3E in buffered solution (400 mM NaCl, 10 mM Hepes pH 8.0, 1 mM ATP, 10 mM MgCl₂, 50 nM fluorescent substrate DNA (SEQ ID NOs: 110, 111 and 112 (labelled with a carboxyfluorescein (FAM) at its 5′ end a black-hole quencher (BHQ-1) at its 3′ end))). The decrease in fluorescence exhibited by Hel308 Mbu(E284C/S615C)-bismaleimidePEG11 (SEQ ID NO: 10 with mutations E284C/S615C connected by a bismaleimidePEG11 linker) and Hel308 Mbu(E284C/S615C)-bismaleimidePEG3 (SEQ ID NO: 10 with mutations E284C/S615C connected by a bismaleimidePEG3 linker), denote the increased processivity of these complexes as compared to Hel308 Mbu monomer (SEQ ID NO: 10). FIG. 6 shows positive controls demonstrating that all helicases were indeed active, as denoted by a fluorescence decrease for all samples.

### Example 4

This example compares the ability of a Hel308 Mbu monomer (SEQ ID NO: 10), to control the movement of intact DNA strands (900 mer) through a nanopore, to that of the Hel308 Mbu(E284C/S615C)-bismaleimidePEG3 (SEQ ID NO. 10 with the following mutations E284C/S615C connected by a bismaleimidePEG3 linker). The general method for controlled DNA translocation against the field is shown in FIG. 7 and with the field in FIG. 8.

### Materials and Methods

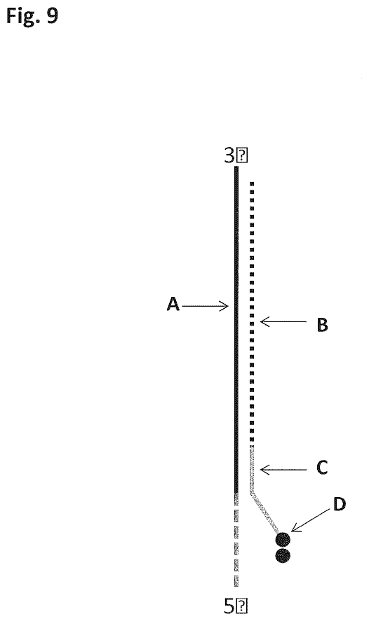

The DNA was formed by ligating a 50-polyT 5′ leader to a ˜900 base fragment of PhiX dsDNA. The leader also contains a complementary section to which SEQ ID NO: 117 (which at the 3′ end of the sequence has six iSp18 spacers attached to two thymine residues and a 3′ cholesterol TEG) was hybridized to allow the DNA to be tethered to the bilayer. Finally the 3′ end of the PhiX dsDNA was digested with AatII digestion enzyme to yield a 4 nt 3′-overhang of ACGT (see FIG. 9 for diagram of the DNA substrate design).
Buffered solution used for Hel308 Mbu: 400 mM NaCl, 100 mM Hepes, 10 mM potassium ferrocyanide, 10 mM potassium ferricyanide pH8.0, 1 mM ATP, 1 mM MgCl₂,
Buffered solution used for Hel308 Mbu(E284C/S615C)-bismaleimidePEG3: 400 mM NaCl, 100 mM Hepes, 10 mM potassium ferrocyanide, 10 mM potassium ferricyanide pH8.0, 2 mM ATP, 2 mM MgCl₂,
Nanopore: E. coli MS(B1-G75S/G77S/L88N/Q126R)8 MspA (SEQ ID NO: 2 with the mutations G75S/G77S/L88N/Q126R)
Enzymes: Hel308 Mbu (SEQ ID NO: 10) added at 200 nM final and Hel308 Mbu(E284C/S615C)-bismaleimidePEG3 (SEQ ID NO: 10 with the following mutations E284C/S615C connected by a bismaleimide3PEG linker) added at 10 nM final.

Electrical measurements were acquired from single MspA nanopores inserted in 1,2-diphytanoyl-glycero-3-phosphocholine lipid (Avanti Polar Lipids) bilayers. Bilayers were formed across ˜100 um diameter apertures in 20 μm thick PTFE films (in custom Delrin chambers) via the Montal-Mueller technique, separating two 1 mL buffered solutions. All experiments were carried out in the stated buffered solution. Single-channel currents were measured on Axopatch 200B amplifiers (Molecular Devices) equipped with 1440A digitizers. Platinum electrodes are connected to the buffered solutions so that the cis compartment (to which both nanopore and enzyme/DNA are added) is connected to the ground of the Axopatch headstage, and the trans compartment is connected to the active electrode of the headstage.

After achieving a single pore in the bilayer, DNA complex (SEQ ID NOs: 115, 116 and 117 (which at the 3′ end of the sequence has six iSp18 spacers attached to two thymine residues and a 3′ cholesterol TEG)), DNA=0.1 nM for the Hel308 Mbu monomer (SEQ ID NO: 10) and 0.05 nM for the Hel308 Mbu(E284C/S615C)-bismaleimidePEG3 (SEQ ID NO: 10 with mutations E284C/S615C connected by a bismaleimidePEG3 linker), MgCl₂ (2 mM) and ATP (2 mM) were added to the cis compartment of the electrophysiology chamber. A control experiment was run at +140 mV. The helicase Hel308 Mbu monomer (SEQ ID NO: 10, 200 nM) or the Hel308 Mbu(E284C/S615C)-bismaleimidePEG3 (SEQ ID NO: 10 with mutations E284C/S615C connected by a bismaleimidePEG3 linker, 10 nM) was then added to the cis compartment. Experiments were carried out at a constant potential of +140 mV.

### Results and Discussion

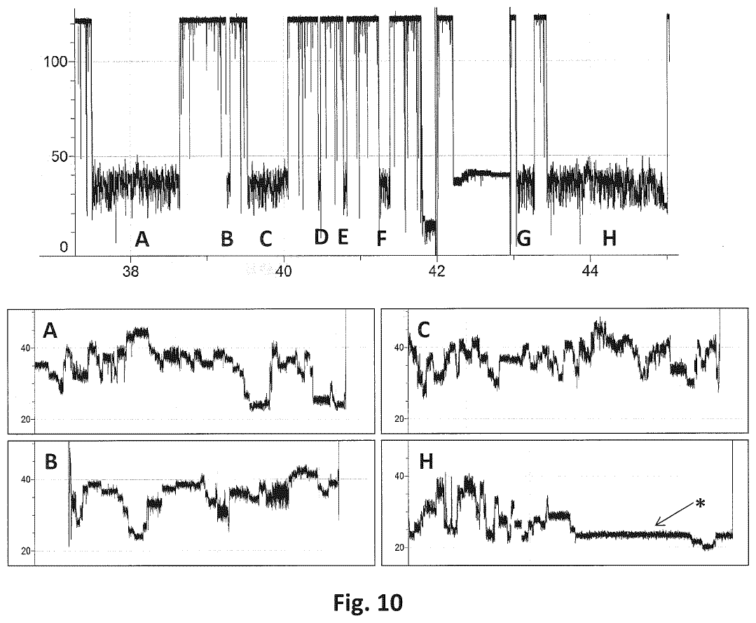

The addition of helicase monomer-DNA substrate to MspA nanopores (as shown in FIG. 7) produces characteristic current blocks as shown in FIG. 10. The helicase Hel308 Mbu monomer (SEQ ID NO: 10) is able to move DNA through a nanopore in a controlled fashion, producing stepwise changes in current as the DNA moves through the nanopore. Example current traces observed when a helicase controls the translocation of DNA (+140 mV, 400 mM NaCl, 100 mM Hepes pH 8.0, 10 mM potassium ferrocyanide, 10 mM potassium ferricyanide, 0.1 nM 900mer DNA (SEQ ID NOs: 115, 116 and 117 (which at the 3′ end of the sequence has six iSp18 spacers attached to two thymine residues and a 3′ cholesterol TEG)), 1 mM ATP, 1 mM MgCl₂) through an MspA nanopore (MS(B1-G75S/G77S/L88N/Q126R)8 MspA (SEQ ID NO: 2 with mutations G75S/G77S/L88N/Q126R) using Hel308 Mbu (200 nM, SEQ ID NO: 10) are shown in FIG. 10. The top electrical trace shows the open pore current (˜120 pA) dropping to a DNA level (20-50 pA) when DNA is captured under the force of the applied potential (+140 mV). DNA with an enzyme attached results in a long block that shows stepwise changes in current as the enzyme moves the DNA through the pore. The upper trace shows a sequence of 8 separate helicase-controlled DNA movements marked A-H (see FIG. 10). All the helicase-controlled DNA movements in this section of trace are being moved through the nanopore against the field by the enzyme (DNA captured 5′down) (see FIG. 7 for details). Below are enlargements of the last section of 4 of the helicase-controlled DNA movements as the DNA exits the nanopore. Of the 8 helicase-controlled DNA movements in this section, only 1 (H) ends in the characteristic long polyT level that indicates that the enzyme has reached the end of the DNA and moved the 50T 5′-leader of the DNA substrate through the pore. In the full run with Hel308 Mbu monomer (SEQ ID NO: 10) it was found that ˜30% of the helicase-controlled DNA movements end at the polyT (n=19 helicase-controlled DNA movements in this experiment).

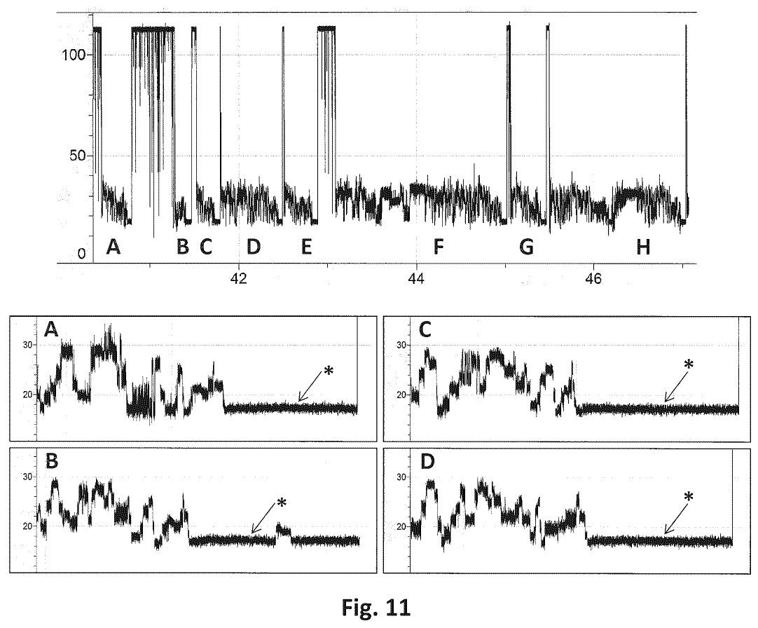

The Hel308 Mbu(E284C/S615C)-bismaleimidePEG3 (10 nM, SEQ ID NO: 10 with mutations E284C/S615C connected by a bismaleimidePEG3 linker) is able to move DNA through a nanopore in a controlled fashion against the field, producing stepwise changes in current as the DNA moves through the nanopore. Example current traces observed when a helicase controls the translocation of DNA (+140 mV, 400 mM NaCl, 100 mM Hepes pH 8.0, 10 mM potassium ferrocyanide, 10 mM potassium ferricyanide, 0.05 nM 900mer DNA (SEQ ID NO: 115, 116 and 117 (which at the 3′ end of the sequence has six iSp18 spacers attached to two thymine residues and a 3′ cholesterol TEG)), 2 mM ATP, 2 mM MgCl₂) through an MspA nanopore (MS(B1-G75S/G77S/L88N/Q126R)8 MspA (SEQ ID NO: 2 with mutations G75S/G77S/L88N/Q126R) using the Hel308 Mbu(E284C/S615C)-bismaleimidePEG3 (10 nM, SEQ ID NO: 10 with mutations E284C/S615C connected by a bismaleimidePEG3 linker) are shown in FIG. 11. The top electrical trace shows the open pore current (˜115 pA) dropping to a DNA level (15-40 pA) when DNA is captured under the force of the applied potential (+140 mV). DNA with enzyme attached results in a long block that shows stepwise changes in current as the enzyme moves the DNA through the pore. The upper trace shows a sequence of 8 separate helicase-controlled DNA movements marked A-H (see FIG. 11). All the helicase-controlled DNA movements in this section of trace are being moved through the nanopore against the field by the enzyme (DNA captured 5′down) (see FIG. 7 for details). Below are enlargements of the last section of 4 of the helicase-controlled DNA movements as the DNA exits the nanopore. Of the 8 helicase-controlled DNA movements in this section, every one ends in the characteristic long polyT level that indicates that the enzyme has reached the end of the DNA and moved the 50T 5′-leader of the DNA substrate through the pore. In the full run with Hel308 Mbu(E284C/S615C)-bismaleimidePEG3 (SEQ ID NO: 10 with mutations E284C/S615C connected by a bismaleimidePEG3 linker) it was found that ˜85% of the helicase-controlled DNA movements against the field (5′ down) end at the polyT (n=27 helicase-controlled DNA movements in this experiment), thus demonstrating substantially improved processivity relative to the unmodified Hel308 Mbu This experiment required only 10 nM enzyme in order to observe helicase-controlled DNA movement, however, Hel308 Mbu monomer (SEQ ID NO: 10) experiments used 200 nM enzyme. Therefore, much lower enzyme concentrations of the helicases in which the opening has been closed can be used while still achieving long read lengths.

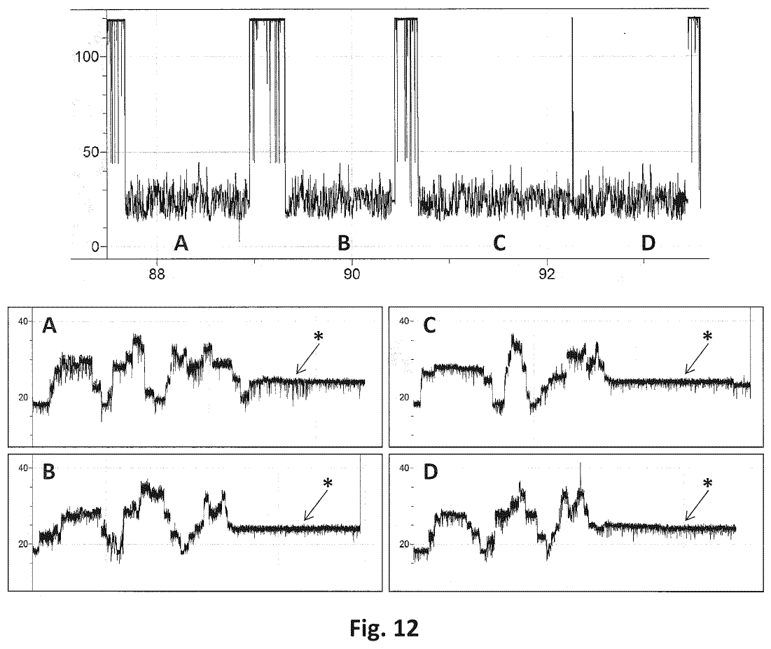

Hel308 Mbu(E284C/S615C)-bismaleimidePEG3 (SEQ ID NO: 10 with mutations E284C/S615C connected by a bismaleimidePEG3 linker) shows enhanced ability to move DNA through a nanopore with the force of the applied field (see FIG. 8 for details), producing stepwise changes in current as the DNA moves through the nanopore. Example current traces observed when a helicase controls the translocation of DNA (+140 mV, 400 mM NaCl, 100 mM Hepes pH 8.0, 10 mM potassium ferrocyanide, 10 mM potassium ferricyanide, 0.05 nM 900mer DNA (SEQ ID NO: 115, 116 and 117 (which at the 3′ end of the sequence has six iSp18 spacers attached to two thymine residues and a 3′ cholesterol TEG)), 2 mM ATP, 2 mM MgCl₂) through an MspA nanopore (MS(B1-G75S/G77S/L88N/Q126R)8 MspA(SEQ ID NO: 2 with mutations G75S/G77S/L88N/Q126R) using the Hel308 Mbu(E284C/S615C)-bismaleimidePEG3 (10 nM, SEQ ID NO: 10 with mutations E284C/S615C connected by a bismaleimidePEG3 linker) are shown in FIG. 12. The top electrical trace shows the open pore current (˜120 pA) dropping to a DNA level (15-40 pA) when DNA is captured under the force of the applied potential (+140 mV). DNA with enzyme attached results in a long block that shows stepwise changes in current as the enzyme moves the DNA through the pore. The upper trace shows a sequence of 4 separate helicase-controlled DNA movements marked A-D (see FIG. 12). All the helicase-controlled DNA movements in this section of trace are being moved through the nanopore with the field by the enzyme (DNA captured 3′down) (see FIG. 8 for details). Below are enlargements of the last section of the helicase-controlled DNA movements as the DNA exits the nanopore. 3′ down DNA shows a characteristically different signature to 5′ down DNA, with a different current to sequence relationship, and different variance. Of the 4 helicase-controlled DNA movements in this section, every one ends in the characteristic long polyT level that indicates that the enzyme has reached the end of the DNA and moved the 50T 5′-leader of the DNA substrate through the pore. In the full run with Hel308 Mbu(E284C/S615C)-bismaleimidePEG3 (SEQ ID NO: 10 with mutations E284C/S615C connected by a bismaleimidePEG3 linker) it was found that ˜87% of helicase-controlled DNA movements with the field (3′ down) end at the polyT (n=15 helicase-controlled DNA movements in this experiment). In comparison, 3′ down helicase-controlled DNA movements are rarely observed when using Hel308 Mbu monomer (SEQ ID NO: 10), and when they are the movements are short with typically less than 50 states observed, indicating a high level of enzyme dissociation in this orientation. The long 3′down helicase-controlled DNA movements, with Hel308 Mbu(E284C/S615C)-bismaleimidePEG3 (SEQ ID NO: 10 with mutations E284C/S615C connected by a bismaleimidePEG3 linker), show a surprising improvement in processivity in the 3′ down mode.

### Example 5

This example shows that the Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)) has the ability to control the movement of intact DNA strands (SEQ ID NO: 127 attached at its 3′ end to four iSpC3 spacers, the last of which is attached to the 5′ end of SEQ ID NO: 128) through a nanopore. The general method for controlled DNA translocation against the field is shown in FIG. 7 and with the field in FIG. 8.

### Materials and Methods

Prior to setting up the experiment, the DNA (0.5 nM, (SEQ ID NO: 127 attached at its 3′ end to four iSpC3 spacers, the last of which is attached to the 5′ end of SEQ ID NO: 128) and Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)) were pre-incubated together for 1 hour.

Electrical measurements were acquired from single MspA nanopores (MS(B1-G75S/G77S/L88N/Q126R)8 MspA (SEQ ID NO: 2 with mutations G75S/G77S/L88N/Q126R) inserted in block co-polymer in buffer (625 mM KCl, 100 mM Hepes, 75 mM Potassium Ferrocyanide (II), 25 mM Potassium ferricyanide (III), pH 8). MgCl₂ (10 mM) and ATP (1 mM) were mixed together with buffer (625 mM KCl, 100 mM Hepes, 75 mM Potassium Ferrocyanide (II), 25 mM Potassium ferricyanide (III), pH 8) and then added to the DNA (SEQ ID NO: 127 attached at its 3′ end to four iSpC3 spacers, the last of which is attached to the 5′ end of SEQ ID NO: 128), Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)) pre-mix. After achieving a single pore in the bilayer, the pre-mix was added to the single nanopore experimental system. Experiments were carried out at a constant potential of +120 mV and helicase-controlled DNA movement was monitored.

### Results and Discussion

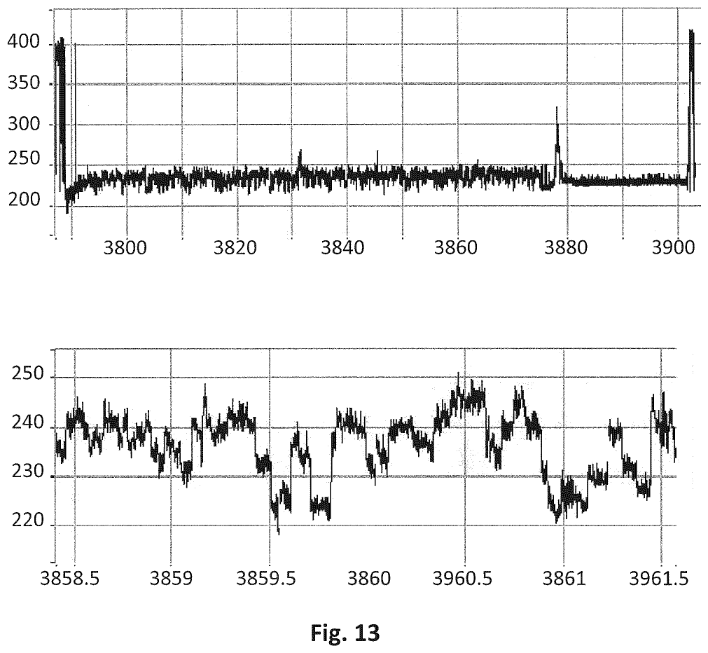

Helicase controlled DNA movement was observed for the closed complex Hel308 Mbu (E284C/S615C)-mal-pep-mal (SEQ ID NO: 10 with the mutations E284C/S615C connected by a bismaleimide peptide linker (maleimide-propyl-SRDFWRS (SEQ ID NO: 109)-(1,2-diaminoethane)-propyl-maleimide)). An example of a helicase-controlled DNA movement is shown in FIG. 13.

### Example 6

This example describes the method of synthesising the TrwC Cba-N691C/Q346C-PEG11 (SEQ ID NO: 126 with the mutations N691C/Q346C connected by a PEG11 linker). In this case a covalent link between cysteines at positions 346 and 691 in the primary sequence of TrwC Cba (SEQ ID NO: 126) was made by reacting these positions with a PEG11 linker.

### Materials and Methods

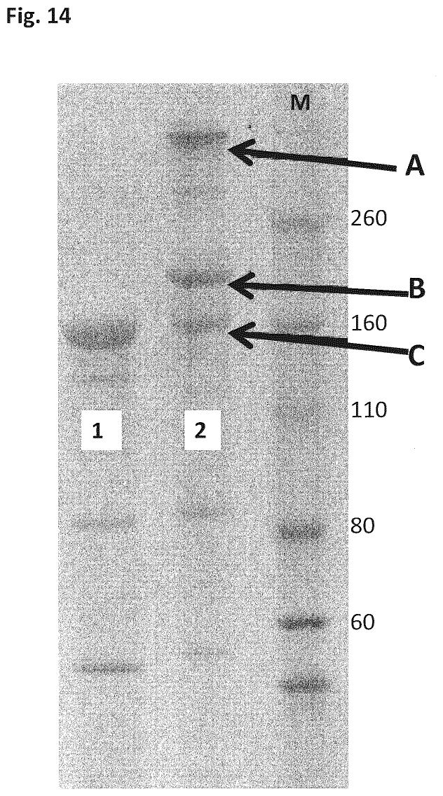

In detail, DTT (2 μl, 1 M) was added to TrwC Cba-N691C/Q346C (200 μl, SEQ ID NO: 126 with the mutations N691C/Q346C, stored in 50 mM Hepes, 10% glycerol, 10 mM DTT, 692 mM NaCl pH7.5) and the mixture was incubated at room temperature on a 10″ wheel rotating at 20 rpm for 30 minutes. This mixture was buffer exchanged through Pierce 2 mL Zeba desalting columns, 7k MWCO into 100 mM potassium phosphate, 500 mM NaCl, 5 mM EDTA, 0.1% Tween-20 pH 8.0 and diluted in the same buffer to give 10 μL aliquots of sample. Maleimide-PEG11-maleimide (50 uM final concentration, Quanta Biodesign, product #10397) was added to one of the aliquots and the mixture incubated at room temperature on a 10″ wheel rotating at 20 rpm for 120 minutes. To stop the reaction, DTT (1 ul of 1 M) was added to quench any remaining maleimides. Analysis of the reaction is by 7.5% polyacrylamide gel. FIG. 14 shows a coomassie stained 7.5% Tris-HCl gel of the TrwC Cba-N691C/Q346C-mal-PEG11-mal (SEQ ID NO: 126 with the mutations N691C/Q346C connected by a bismaleimide polyethylene glycol linker) reaction mixture. The lane on the right of the gel (labelled M) shows an appropriate protein ladder (the mass unit markers are shown on the right of the gel). Lane 1 contains 5 μL of approximately 10 μM TrwC Cba-D657C/R339C alone (SEQ ID NO: 126 with mutation D657C/R339C) as a reference. Lane 2 contains 5 μL of approximately 10 μM TrwC Cba-N691C/Q346C-bismaleimdiePEG11 (SEQ ID NO: 126 with the mutations N691C/Q346C connected by a bismaleimide PEG11 linker). As indicated in lane 2, the upper band corresponds to the dimeric enzyme species (labelled A), the middle band corresponds to the closed complex (labelled B) TraI-Cba-N691C/Q346C-bismaleimidePEG11 (SEQ ID NO: 126 with the mutations N691C/Q346C connected by a bismaleimide PEG11 linker). It was clear from the gel that the reaction to attach the mal-PEG11-mal linker did not go to completion as a band for unmodified starting material (labelled C) TrwC Cba-N691C/Q346C (SEQ ID NO: 126 with the mutations N691C/Q346C) was observed.

The TrwC Cba-N691C/Q346C-PEG11 (SEQ ID NO: 126 with the mutations N691C/Q346C connected by a PEG11 linker) was then buffer exchanged to 50 mM Tris, 500 mM NaCl, 2 mM DTT, pH 8.0.

Using an analogous procedure to that described in this example, it was possible to make the following closed complexes listed in Table 10 below.

TABLE 10

| Entry | | |
|---|---|---|
| No. | Closed complex | Sequence |
| 1 | TrwC Cba-N691C/Q346C- | SEQ ID NO: 126 with the mutations N691C/ |
| | mal-pep-mal | Q346C connected by a bismaleimide peptide |
| | | linker (maleimide-propyl-SRDFWRS (SEQ |
| | | ID NO: 109)-(1,2-diaminoethane)-propyl- |
| | | maleimide) |
| 2 | TrwC Cba-N691C/Q346C- | SEQ ID NO: 126 with the mutations N691C/ |
| | bismaleimidePEG3 | Q346C connected by a bismaleimide |
| | | PEG3 linker |
| 3 | TrwC Cba-D657C/R339C- | SEQ ID NO: 126 with the mutations D657C/ |
| | mal-pep-mal | R339C connected by a bismaleimide peptide |
| | | linker (maleimide-propyl-SRDFWRS (SEQ |
| | | ID NO: 109)-(1,2-diaminoethane)-propyl- |
| | | maleimide) |
| 4 | TrwC Cba-D657C/R339C- | SEQ ID NO: 126 with the mutations D657C/ |
| | bismaleimidePEG3 | R339C connected by a bismaleimide |
| | | PEG3 linker |
| 5 | TrwC Cba-D657C/R339C- | SEQ ID NO: 126 with the mutations D657C/ |
| | bismaleimidePEG11 | R339C connected by a bismaleimide |
| | | PEG11 linker |
| 6 | TrwC Cba-N691C/S350C- | SEQ ID NO: 126 with the mutations N691C/ |
| | mal-pep-mal | S350C connected by a bismaleimide peptide |
| | | linker (maleimide-propyl-SRDFWRS (SEQ |
| | | ID NO: 109)-(1,2-diaminoethane)-propyl- |
| | | maleimide) |
| 7 | TrwC Cba-N691C/S350C- | SEQ ID NO: 126 with the mutations N691C/ |
| | bismaleimidePEG3 | S350C connected by a bismaleimide |
| | | PEG3 linker |
| 8 | TrwC Cba-N691C/S350C- | SEQ ID NO: 126 with the mutations N691C/ |
| | bismaleimidePEG11 | S350C connected by a bismaleimide |
| | | PEG11 linker |
| 9 | TrwC Cba-V690C/S350C- | SEQ ID NO: 126 with the mutations V690C/ |
| | mal-pep-mal | S350C connected by a bismaleimide peptide |
| | | linker (maleimide-propyl-SRDFWRS (SEQ |
| | | ID NO: 109)-(1,2-diaminoethane)-propyl- |
| | | maleimide) |
| 10 | TrwC Cba-V690C/S350C- | SEQ ID NO: 126 with the mutations V690C/ |
| | bismaleimidePEG3 | S350C connected by a bismaleimide |
| | | PEG3 linker |
| 11 | TrwC Cba-V690C/S350C- | SEQ ID NO: 126 with the mutations V690C/ |
| | bismaleimidePEG11 | S350C connected by a bismaleimide |
| | | PEG11 linker |

### Example 7

This Example illustrates that when a number of helicases were investigated (Hel308 Mbu (SEQ ID NO: 10), Hel308 Mbu-E284C (SEQ ID NO: 10 with the mutation E284C), Hel308 Mbu-E284C/C301A (SEQ ID NO: 10 with the mutations E284C/C301A), Hel308 Mbu-E285C (SEQ ID NO: 10 with the mutation E285C), Hel308 Mbu-S288C (SEQ ID NO: 10 with the mutation S288C), and Hel308 Mbu-D274C (SEQ ID NO: 10 with the mutation D274C) for their rate of turnover of dsDNA molecules (min⁻¹enzyme⁻¹) using a fluorescent assay, the mutant helicases (Hel308 Mbu-E284C (SEQ ID NO: 10 with the mutation E284C), Hel308 Mbu-E284C/C301A (SEQ ID NO: 10 with the mutations E284C/C301A), Hel308 Mbu-E285C (SEQ ID NO: 10 with the mutation E285C), Hel308 Mbu-S288C (SEQ ID NO: 10 with the mutation S288C), and Hel308 Mbu-D274C (SEQ ID NO: 10 with the mutation D274C)) tested had increased rate of turnover of dsDNA molecules (min⁻¹enzyme⁻¹) in comparison to Hel308 Mbu (SEQ ID NO: 10).

### Materials and Methods

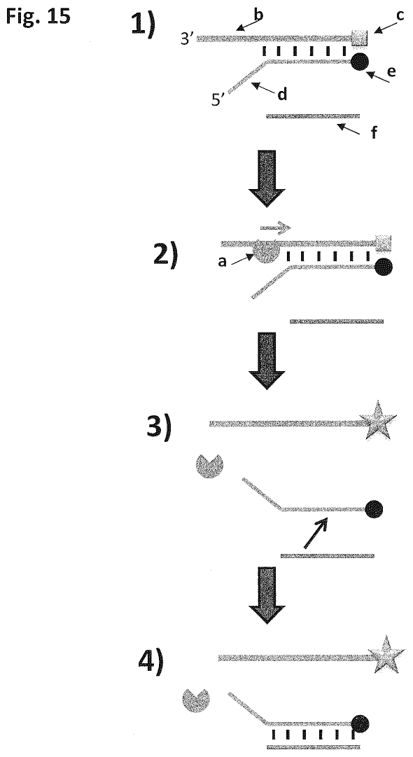

A custom fluorescent substrate was used to assay the ability of a number of Hel308 Mbu helicases (Hel308 Mbu-E284C (SEQ ID NO: 10 with the mutation E284C), Hel308 Mbu-E284C/C301A (SEQ ID NO: 10 with the mutations E284C/C301A), Hel308 Mbu-E285C (SEQ ID NO: 10 with the mutation E285C), Hel308 Mbu-S288C (SEQ ID NO: 10 with the mutation S288C), and Hel308 Mbu-D274C (SEQ ID NO: 10 with the mutation D274C)) to displace hybridised dsDNA. As shown in 1) of FIG. 15, the fluorescent substrate strand (50 nM final) has both a 3′ and 5′ ssDNA overhang, and a 44 base section of hybridised dsDNA. The upper strand, containing the 3′ ssDNA overhang, has a carboxyfluorescein base (the carboxyfluorescein (labelled c in FIG. 15) is attached to a thymine at position 6 in SEQ ID NO: 151) at the 5′ end, and the hybrised complement has a black-hole quencher (BHQ-1, labelled e in FIG. 15) base (the black-hole quencher is attached to a thymine at position 81 in SEQ ID NO: 152) at the 3′ end. When the two strands are hybridised the fluorescence from the fluorescein is quenched by the local BHQ-1, and the substrate is essentially non-fluorescent. 1 μM of a capture strand (SEQ ID NO: 153) that is part-complementary to the lower strand of the fluorescent substrate is included in the assay. As shown in 2), in the presence of ATP (1 mM) and MgCl₂ (10 mM), appropriate helicase (10 nM) added to the substrate binds to the 3′ tail of the fluorescent substrate, moves along the upper strand, and displaces the complementary strand. As shown in 3), once the complementary strand with BHQ-1 is fully displaced the fluorescein on the major strand fluoresces. As shown in 4) the displaced strand preferentially anneals to an excess of capture strand to prevent re-annealing of initial substrate and loss of fluorescence.

### Results and Discussion

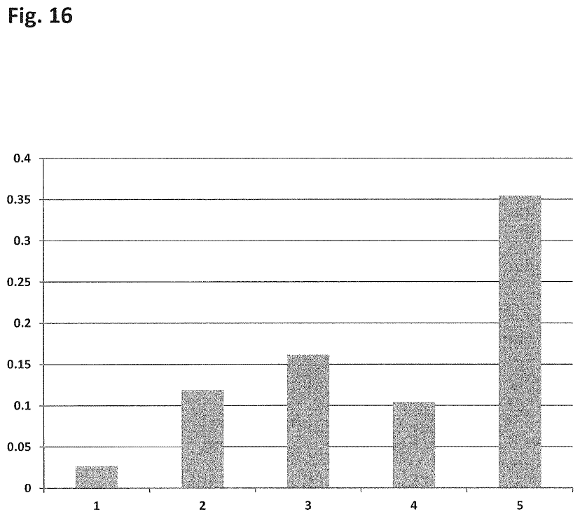

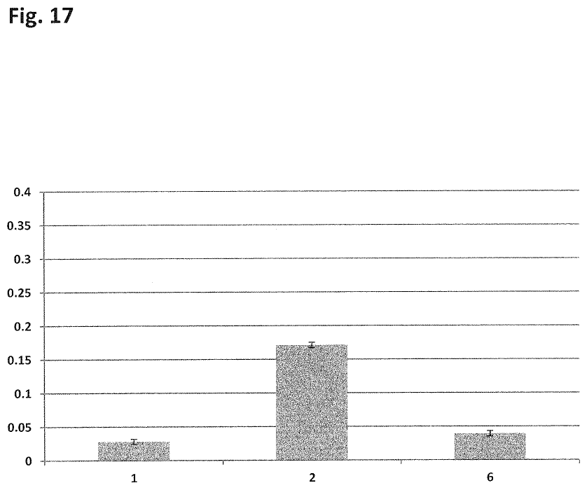

The graphs in FIGS. 16 and 17 show the dsDNA turnover (enzyme⁻¹min⁻¹) in buffer (400 mM KCl, 100 mM Hepes pH 8.0, 1 mM ATP, 10 mM MgCl₂, 50 nM fluorescent substrate DNA (SEQ ID NOs: 151 and 152), 1 μM capture DNA (SEQ ID NO: 153)) for a number of helicases (Hel308 Mbu (labelled 1 in FIGS. 16 and 17, SEQ ID NO: 10), Hel308 Mbu-E284C (labelled 2 in FIGS. 16 and 17, SEQ ID NO: 10 with the mutation E284C), Hel308 Mbu-E284C/C301A (labelled 3 in FIG. 16, SEQ ID NO: 10 with the mutations E284C/C301A), Hel308 Mbu-E285C (labelled 4 in FIG. 16, SEQ ID NO: 10 with the mutation E285C), Hel308 Mbu-S288C (labelled 5 in FIG. 16, SEQ ID NO: 10 with the mutation S288C) and Hel308 Mbu-D274C (labelled 6 in FIG. 17, SEQ ID NO: 10 with the mutation D274C)). At the salt concentration investigated (400 mM KCl) the following helicases Hel308 Mbu-E284C (FIGS. 16 and 17 labelled 2, SEQ ID NO: 10 with the mutation E284C), Hel308 Mbu-E284C/C301A (FIG. 16 labelled 3, SEQ ID NO: 10 with the mutations E284C/C301A), Hel308 Mbu-E285C (FIG. 16 labelled 4, SEQ ID NO: 10 with the mutation E285C), Hel308 Mbu-S288C (FIG. 16 labelled 5, SEQ ID NO: 10 with the mutation S288C) and Hel308 Mbu-D274C (FIG. 17 labelled 6, SEQ ID NO: 10 with the mutation D274C) exhibited a higher rate of dsDNA turnover than the control Hel308 Mbu (FIGS. 16 and 17 labelled 1, SEQ ID NO: 10) (see FIGS. 16 and 17). This indicates that these enzymes show increased rate of turnover of dsDNA molecules (min⁻¹enzyme⁻¹) when compared to the Hel 308 Mbu control (SEQ ID NO: 10) under the conditions investigated.

### Example 8

This example describes two procedures for the light treatment of Hel308 Mbu-E284Faz (SEQ ID NO: 10 with the mutation E284Faz) and Hel308 Mbu-S288Faz (SEQ ID NO: 10 with the mutation S288Faz).

### Procedure 1—Exposure to UV Light

Hel308 Mbu-E284Faz (SEQ ID NO: 10 with the mutation E284Faz) or Hel308 Mbu-S288Faz (SEQ ID NO: 10 with the mutation E288Faz) in storage buffer (50 mM Tris pH8.0 at 4° C., NaCl (360-390 mM) and 5% Glycerol) was pipetted into PCR tubes (Fisher 0.2 mL thin wall tubes). The sample was placed on ice and exposed to high intensity UV light at 254 nm (Spectroline Longlife Filter lamp (254 nm and 365 nm) from above, at a distance of 4.5 cm. The Hel308 Mbu-E284Faz (SEQ ID NO: 10 with the mutation E284Faz) sample was exposed for 15 mins and the Hel308 Mbu-S288Faz (SEQ ID NO: 10 with the mutation E288Faz) sample was exposed for 10 mins. The samples were then both centrifuged for 5 mins at 16 000 g to remove any precipitated protein. The soluble fraction was carefully removed from the insoluble pellet by pipette.

### Procedure 2—Exposure to White Light (LED Source)

Hel308 Mbu-S288Faz (SEQ ID NO: 10 with the mutation E288Faz) in storage buffer (50 mM Tris pH8.0 at 4° C., NaCl (370 mM) and 5% Glycerol) was pipetted into Microcentrifuge tube (Eppendorf, 1.5 mL, Protein Lo Bind). The sample was placed on ice (with the cap open) and exposed to LED light source (Schott A20960.1) on full power from above, at a distance of 3 cm. The Hel308 Mbu-S288Faz (SEQ ID NO: 10 with the mutation E288Faz) sample was exposed for 3 hours.

### Procedure 3—Exposure to White Light (LED Source) and Heating

Hel308 Mbu-S288Faz (SEQ ID NO: 10 with the mutation E288Faz) in storage buffer (50 mM Tris pH8.0 at 4° C., NaCl (370 mM) and 5% Glycerol) was pipetted into Microcentrifuge tube (Eppendorf, 1.5 mL, Protein Lo Bind). The sample was placed on ice (with the cap open) and exposed to LED light source (Schott A20960.1) on full power from above, at a distance of 1 cm. The Hel308 Mbu-S288Faz (SEQ ID NO: 10 with the mutation E288Faz) sample was exposed for 1 hour. The sample was transferred in a PCR tube (Fisher 0.2 mL thin wall tube) and heated at 50° C. for 10 min before ramping to 4° C., then centrifuged for 5 mins at 16 000 g to remove any precipitated protein. The soluble fraction was carefully removed from the insoluble pellet by pipette.

### Example 9

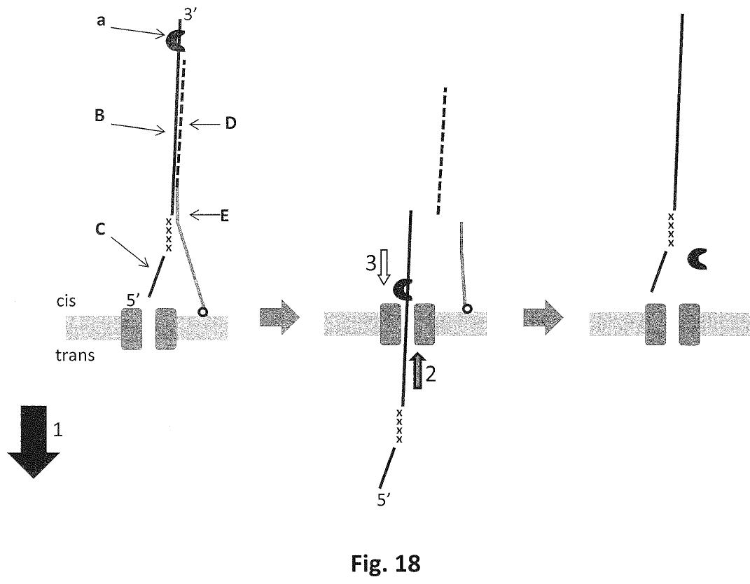

This example compares the ability of Hel308 Mbu (SEQ ID NO: 10), to control the movement of intact DNA strands (3.6 kb) through a nanopore, to that of a number of Hel308 Mbu mutants (Hel308 Mbu-E284C (SEQ ID NO: 10 with the mutation E284C), Hel308 Mbu-S288C (SEQ ID NO: 10 with the mutation S288C), Hel308 Mbu-E284Faz (SEQ ID NO: 10 with the mutation E284Faz) and heat treated Hel308 Mbu-E284Faz (SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block)). The general method for controlled DNA translocation against the field is shown in FIG. 18.

### Materials and Methods

Prior to setting up the experiment, the DNA (0.2 nM, (SEQ ID NO: 154 attached at its 5′ end to four nitroindoles (labelled as x's in FIG. 18), the last of which is attached to the 3′ end of SEQ ID NO: 155), SEQ ID NO: 156 and SEQ ID NO: 117) and appropriate helicase (Hel 308 Mbu (100 nM, SEQ ID NO: 10), Hel308 Mbu-E284C (100 nM, SEQ ID NO: 10 with the mutation E284C), Hel308 Mbu-S288C (100 nM, SEQ ID NO: 10 with the mutation S288C), Hel308 Mbu-E284Faz (100 nM, SEQ ID NO: 10 with the mutation E284Faz) and heat treated Hel308 Mbu-E284Faz (500 nM, SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block)) were dissolved in buffer (960 mM KCl, 25 mM potassium phosphate, 3 mM potassium ferrocyanide, 1 mM potassium ferricyanide pH 8.0, 10 mM MgCl₂ and 1 mM ATP).

Electrical measurements were acquired from single MspA nanopores (MS(B1-G75S/G77S/L88N/Q126R)8 MspA (SEQ ID NO: 2 with mutations G75S/G77S/L88N/Q126R) inserted in block co-polymer in buffer (960 mM KCl, 25 mM potassium phosphate, 3 mM potassium ferrocyanide, 1 mM potassium ferricyanide pH 8.0). After achieving a single pore in the block co-polymer, buffer (3 mL of 960 mM KCl, 25 mM potassium phosphate, 3 mM potassium ferrocyanide, 1 mM potassium ferricyanide pH 8.0) was then flowed through the system. Finally, the pre-mix (described above) was added to the single nanopore experimental system. Experiments were carried out at a constant potential of +120 mV and helicase-controlled DNA movement was monitored.

### Results and Discussion

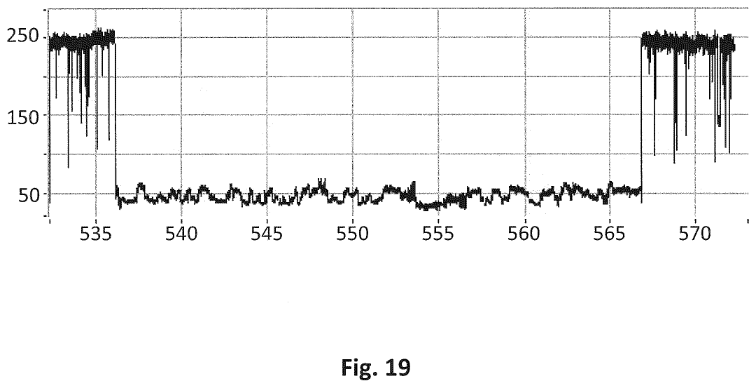

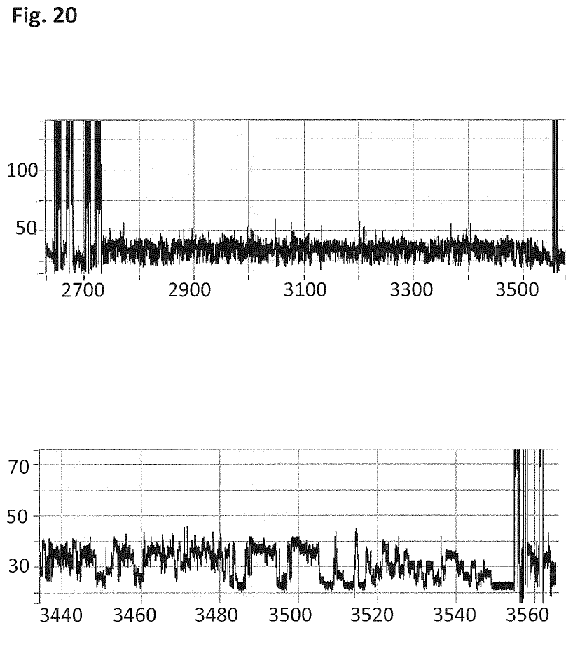

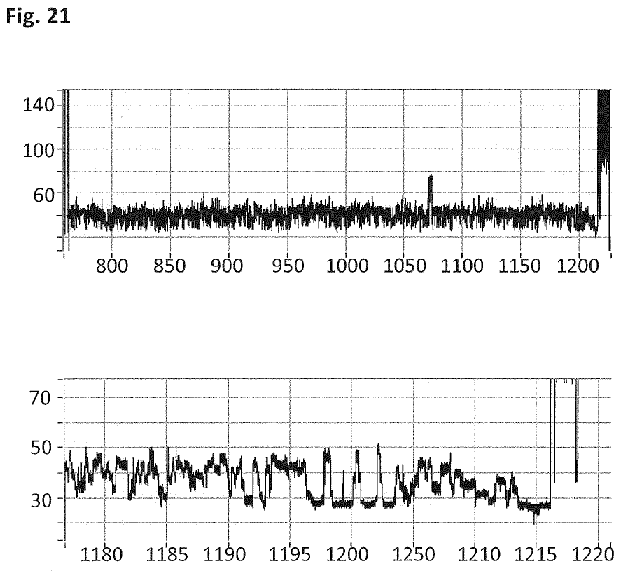

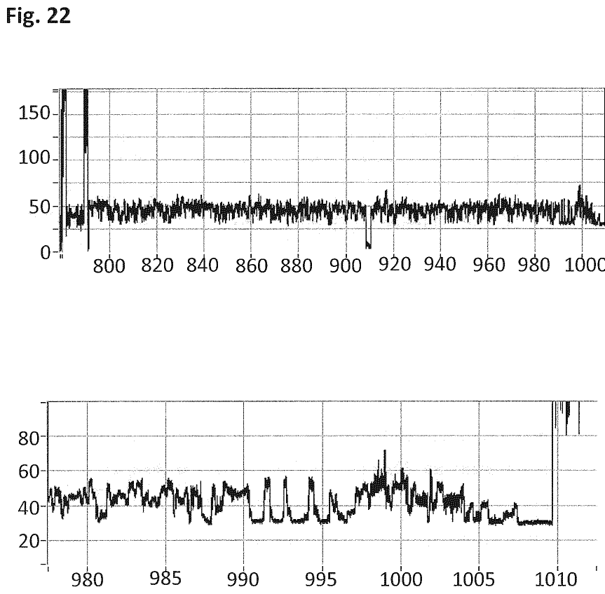

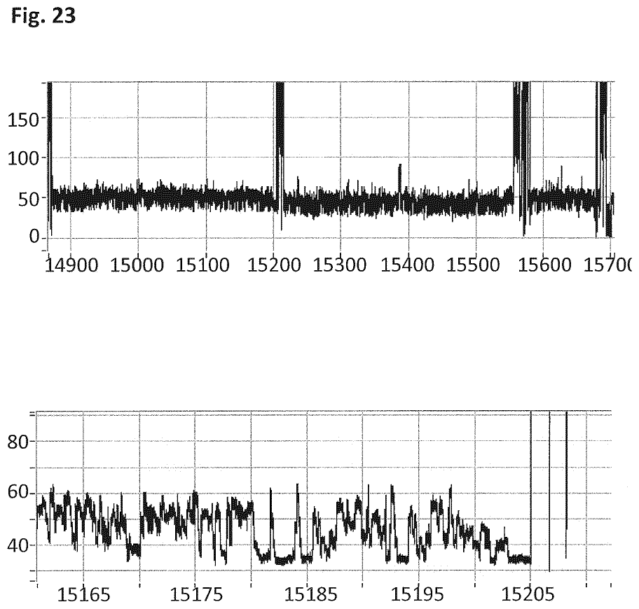

Helicase controlled DNA movement was observed for all of the enzymes tested—Hel 308 Mbu (SEQ ID NO: 10), Hel308 Mbu-E284C (SEQ ID NO: 10 with the mutation E284C), Hel308 Mbu-S288C (SEQ ID NO: 10 with the mutation S288C), Hel308 Mbu-E284Faz (SEQ ID NO: 10 with the mutation E284Faz) and heat treated Hel308 Mbu-E284Faz (SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block). Example current traces showing helicase controlled DNA movement are shown in FIGS. 19-23. However, the mutant Hel308 Mbu helicases (Hel308 Mbu-E284C (SEQ ID NO: 10 with the mutation E284C), Hel308 Mbu-S288C (SEQ ID NO: 10 with the mutation S288C), Hel308 Mbu-E284Faz (SEQ ID NO: 10 with the mutation E284Faz) and heat treated Hel308 Mbu-E284Faz (SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block)) showed increased processivity in comparison to Hel308 Mbu (SEQ ID NO: 10) see Table 11. Of the helicase controlled DNA movements observed in the experiments, the % of movements which processed the DNA all the way to the end of the strand (to the polyT region) were significantly higher for the mutant helicases (Hel308 Mbu-E284C (SEQ ID NO: 10 with the mutation E284C), Hel308 Mbu-S288C (SEQ ID NO: 10 with the mutation S288C), Hel308 Mbu-E284Faz (SEQ ID NO: 10 with the mutation E284Faz) and heat treated Hel308 Mbu-E284Faz (SEQ ID NO: 10 with the mutation E284Faz, the enzyme was heated in 50 mM Tris pH 8.0, 375 mM NaCl, 5% Glycerol buffer from 4° C. to 50° C. for 10 mins and then cooled to 4° C. in a BioRad PCR block)) when compared to Hel308 Mbu (SEQ ID NO: 10).

TABLE 11

| | % of Helicase |
|---|---|
| | Controlled DNA |
| | movement that reached |
| | the polyT region |
| | of the DNA strand |
| | (SEQ ID NO: 154 |
| | attached at its 5′ |
| | end to four nitroindoles |
| | the last of which is |
| | attached to the 3′ end |
| Helicase | of SEQ ID NO: 155) |
| Hel308 Mbu (SEQ ID | 2 |
| NO: 10) | |
| Hel308 Mbu-E284C | 32 |
| (SEQ ID NO: 10 | |
| with the mutation | |
| E284C) | |
| Hel308 Mbu-E288C | 49 |
| (SEQ ID NO: 10 | |
| with the mutation | |
| E288C) | |
| Hel308 Mbu-E284Faz | 28 |
| (SEQ ID NO: 10 | |
| with the mutation | |
| E284Faz) | |
| Heat treated Hel308 Mbu- | 71 |
| E284Faz (SEQ ID NO: | |
| 10 with the mutation | |
| E284Faz, the enzyme was | |
| heated in 50 mM Tris | |
| pH 8.0, 375 mM NaCl, | |
| 5% Glycerol buffer | |
| from 4° C. to 50° C. | |
| for 10 mins and then | |
| cooled to 4° C. in a | |
| BioRad PCR block) | |

1.-30. (canceled)

31. A method of controlling the movement of a polynucleotide through a transmembrane pore, the method comprising:
(a) contacting the polynucleotide with the transmembrane pore and a helicase that comprises a polynucleotide binding domain, wherein two amino acid residues that are located on a surface of the helicase are covalently connected via a linkage between the two amino acid residues to form a covalently-closed structure, thereby controlling the movement of the polynucleotide through the transmembrane pore.

32. The method of claim 31 further comprising:
(b) taking one or more measurements as the polynucleotide moves with respect to the transmembrane pore, wherein the one or more measurements are indicative of one or more characteristics of the polynucleotide.

33. The method of claim 32, wherein the one or more characteristics are selected from (i) the length of the polynucleotide, (ii) the identity of the polynucleotide, (iii) the sequence of the polynucleotide, (iv) the secondary structure of the polynucleotide and (v) whether or not the polynucleotide is modified

34. The method of claim 33, wherein the polynucleotide is modified by methylation, by oxidation, by damage, with one or more proteins or with one or more labels, tags or spacers.

35. The method of claim 32, wherein the one or more characteristics of the polynucleotide are measured by electrical measurement and/or optical measurement.

36. The method of claim 35, wherein the electrical measurement is a current measurement, an impedance measurement, a tunnelling measurement or a field effect transistor (FET) measurement.

37. (canceled)

38. The method of claim 31, wherein the method further comprises the step of applying a voltage across the transmembrane pore.

39. The method of claim 31, wherein at least a portion of the polynucleotide is double stranded.

40. (canceled)

41. The method of claim 31, wherein the transmembrane pore is derived from a hemolysin, leukocidin, Mycobacterium smegmatis porin A (MspA), MspB, MspC, MspD, outer membrane porin F (OmpF), outer membrane porin G (OmpG), outer membrane phospholipase A, Neisseria autotransporter lipoprotein (NalP) and WZA.

42.-59. (canceled)

60. The method of claim 31, wherein at least one amino acid of the two amino acid residues is substituted with cysteine, a non-natural amino acid or 4-azido-L-phenylalanine (Faz).

61. The method of claim 31, wherein the two amino acid residues that are located on the surface of the helicase are on one or more loop regions connecting α-helices and β-strands of the helicase and/or are spatially located proximal to the polynucleotide binding domain.

62. The method of claim 31, wherein the helicase is (a) a Hel308 helicase, a RecD helicase, a TraI helicase, a TraI subgroup helicase, an XPD helicase; or (b) one of the helicases as set forth in SEQ ID NO: 10, 13, 16, 19, 22, 25, 28, 29, 32-34, 37-55, 58, 85, 126, 134, 138.

63. The method of claim 31, wherein the helicase further comprises a second set of two amino acid residues in different structural domains of the helicase surrounding the polynucleotide binding domain that are covalently connected via a linkage between the two amino acid residues of the second set.

64. The method of claim 31, wherein the linkage between the two amino acid residues comprises a peptide linker.

65. The method of claim 64, wherein the peptide linker comprises a polynucleotide binding moiety.

66. The method of claim 31, wherein the linkage between the two amino acid residues comprises a polyethyleneglycol (PEG), polysaccharide, polyamide, deoxyribonucleic acid (DNA) sequence, peptide nucleic acid (PNA), threose nucleic acid (TNA), or glycerol nucleic acid (GNA).

67. The method of claim 31, wherein the helicase is a member of a helicase family selected from the group consisting of: Pif1-like helicases, Upf1-like helicases, UvrD/Rep helicases, Ski-like helicases, Rad3/XPD helicases, NS3/NPH-II helicases, DEAD helicases, DEAHi RHA helicases, RecG-like helicases, REcQ-like helicases, T1R-like helicases, Swi/Snf-like helicases, and Rig-I-like helicases.

68. The method of claim 31, wherein the helicase is a member of a helicase subfamily selected from the group consisting of: RecD helicases, Upf1 helicases, PcrA helicases, Rep helicases, UvrD helicases, Hel308 helicases, Mtr4 helicases, XPD helicases, NS3 helicases, Mssl 16 helicases, Prp43 helicases, RecG helicases, RecQ helicases, T1R helicases, RapA helicases and Hef helicases.

69. The method of claim 31, wherein the transmembrane pore is a β-barrel pore or an α-helix bundle pore.

70. The method of claim 31, wherein at least one amino acid of the two amino acid residues is a cysteine that has been introduced into the helicase.
